(19) United States
(12) Patent Application Publication
(10) Pub. No.: US 2024/0043894 A1
(43) Pub. Date: Feb. 8, 2024

(54) PRODUCTION OF NMN AND ITS DERIVATIVES VIA MICROBIAL PROCESSES

(71) Applicant: Conagen Inc., Bedford, MA (US)

(72) Inventors: David Nunn, Lowell, MA (US); Jacob Edward Vick, Stoneham, MA (US); Bryan Salas-Santiago, Lowell, MA (US)

(73) Assignee: Conagen Inc., Bedford, MA (US)

(21) Appl. No.: 18/052,595

(22) Filed: Nov. 4, 2022

Related U.S. Application Data
(63) Continuation of application No. PCT/US2021/030601, filed on May 4, 2021.
(60) Provisional application No. 63/020,052, filed on May 5, 2020.

(51) Int. Cl. C12P 19/36; C12P 19/30; C12N 9/12; C12N 9/10; C12N 9/24; C12N 9/02; C12N 9/14; C12N 9/78

(52) U.S. Cl. CPC C12P 19/36; C12P 19/30; C12N 9/1235; C12Y 207/06001; C12N 9/1077; C12Y 204/02012; C12N 9/1241; C12Y 207/07018; C12N 9/2497; C12Y 302/02001; C12N 9/0036; C12Y 106/01001; C12N 9/14; C12Y 306/01022; C12N 9/78; C12Y 305/01019

(57) ABSTRACT

The present invention relates to microbial production of nicotinamide mononucleotide (NMN), nicotinamide riboside (NR), and nicotinamide adenine dinucleotide (NAD) using a genetically modified bacterium.

### RELATED APPLICATIONS

This application claims the benefit under 35 U.S.C. § 119(e) to U.S. Provisional Application No. 63/020,052, filed May 5, 2020, and entitled “PRODUCTION OF NMN AND ITS DERIVATIVES VIA MICROBIAL PROCESSES,” the entire contents of which is incorporated herein by reference.

### FIELD OF THE INVENTION

The field of the invention relates to the production of nicotinamide mononucleotide (NMN) and its derivatives including nicotinamide riboside (NR) and nicotinamide adenine dinucleotide (NAD) via microbial processes.

### BACKGROUND OF THE INVENTION

In recent years, nicotinamide adenine dinucleotide (NAD) has evolved from being a simple metabolic cofactor to the status of a therapeutic agent in health care applications (Katsyuba et al 2020—NAD⁺ homeostasis in health and disease. Nature Metabolism 2: 9-31). Similarly, two other compounds both closely related to NAD, namely nicotinamide mononucleotide (NMN) and nicotinamide riboside (NR), are currently recognized as nutraceuticals with potential in anti-aging and longevity applications. NMN and NR are currently produced commercially using chemical methods and there is a need for developing bioprocess technology to manufacture these compounds at commercial scales in a cost-effective way.

The bacterium Corynebacterium stationis (ATCC 6872 and variously described as Brevibacterium ammoniagenes, Corynebacterium ammoniagenes and Brevibacterium stationis) has been found to be a prolific producer of NMN and related compounds when grown under restrictive conditions. Typical restrictive conditions include starvation for manganese, addition of specific chemical inhibitors and, with specific temperature-sensitive mutants, culturing at high temperatures. Included in this stress-induced microbial production of NMN is nicotinate mononucleotide (NAMN), a compound related to NMN when the cells are grown under restrictive conditions and fed with either nicotinic acid (NA) or nicotinamide (Nam). There remains a need in the art for production methods of NMN that increase the amount of NMN produced while minimizing or eliminating the formation of side product NAMN.

### SUMMARY OF THE INVENTION

The present invention addresses the need described above by providing genetic modifications to Corynebacterium glutamicum that enable the production of nicotinamide mononucleotide (NMN) rather than nicotinate mononucleotide (NAMN), as well as the production of NMN-derived molecules such as NR and NAD.

The present invention relates to engineered host cells with genetic modifications that enable the production of NMN and its derivatives NR and NAD. Also provided in this invention are methods to genetically engineer microbial host cells capable of selectively producing either NMN or NR or NAD. The feedstocks useful for the production of NMN, NR and NAD according to the present invention include nicotinamide (Nam) and nicotinic acid (NA). In a preferred embodiment of the present invention, Nam is used as the feedstock for the production of NMN, NAR or NAD.

The microbial host cells useful for the production of NMN. NR and/or NAD according to the present invention include, but are not limited to, microbial cells such as bacterial cells, fungal cells and yeast cells amenable to genetic modifications. In one aspect of the present invention, Corynebacterium glutamicum is used as the preferred microbial host cell. In a preferred aspect of this invention, Corynebacterium glutamicum cells useful for this invention are initially subjected to genetic modifications to alter its restriction modification system to make it suitable for other genetic modifications which enable to use this bacterial cell for producing NMN, NR and NAD using Nam or NA a feedstock. The present engineered host cells, including Corynebacterium glutamicum, may comprise genetic modifications for introducing a conditionally active ribonucleotide reductase to block cell division and biomass accumulation without affecting protein synthesis and normal metabolic activity, e.g., a ribonucleotide reductase coded by a NrdHIEJ operon.

In one aspect of the present invention, the genetic modifications suitable for the production of NMN, NR and/or NAD include, but are not limited to, introduction of an exogenous gene expressing an enzyme protein not originally present in the microbial host cell, increasing the relative expression of an endogenous gene coding for an enzyme protein leading to an increase in the activity of the corresponding enzyme protein, and/or decreasing or totally eliminating the expression of an endogenous gene coding for an enzyme protein causing a decrease or total elimination of the activity of the corresponding enzyme protein.

In one aspect of the present invention, where the engineered microbial host cell lacks an endogenous gene coding for an enzyme capable of converting Nam to NMN, an exogenous gene nadV coding for nicotinamide phosphoribosyl transferase enzyme is introduced into the engineered microbial host cell. In one exemplary embodiment of the present invention, the source of nadV gene coding for nicotinamide phosphoribosyl transferase enzyme includes, but is not limited to, Stenotrophomonas maltophilia, Chromobacterium violaceum, Deinococcus radiodurans, Synechocystis sp. PCC 6803, Pseudonocardia dioxanivorans CB1190, Shewanella oneidensis MR-1 and Ralstonia solanacearum GMI1000. In a most preferred aspect of the present invention, an nadV gene from any one of the foregoing microbial species is isolated and introduced into a strain of Corynebacterium glutamicum using one or more genetic engineering techniques well known in the art. In a preferred aspect, the exogenous gene nadV is codon optimized before its transfer into the engineered microbial host cell to assure optimal expression of the nicotinamide phosphoribosyl transferase enzyme in the microbial host cell. The exogenous gene within the Corynebacteriun glutamicum cell may be under an inducible promoter such as lacZ promoter and can be induced using lactose or IPTG.

In another aspect of the present invention, the NAMPT gene from Haemophilus ducreyi is introduced into the engineered microbial host cell as a source of nicotinamide phosphoribosyl transferase enzyme activity. In yet another aspect of this embodiment, in those microbial host cells already having an endogenous gene coding for nicotinamide phosphoribosyl transferase enzyme, the activity of this enzyme may be further enhanced by means of introducing an exogenous nadV gene (optionally codon-optimized and obtained from any of the sources listed in the foregoing paragraph) or an optionally codon optimized NAMPT gene from Haemophilus ducreyi.

In another aspect of the present embodiment, the microbial host cell selected for producing NMN using Nam as a feedstock and having a codon optimized exogenous gene coding nicotinamide phosphoribosyl transferase enzyme further comprises an additional genetic modification to assure the availability of phosphoribosyl pyrophosphate (PRPP also known as 5-phospho-ribose 1-diphosphate) serving as a co-substrate in the enzymatic reaction leading to the conversion of Nam to NMN. The additional genetic modification includes an introduction of an exogenous gene such as prsA, coding for the phosphoribosyl pyrophosphate synthetase or improving the performance of endogenous prsA gene either by increasing its expression or improving the performance of the phosphoribosyl pyrophosphate synthetase enzyme PrsA coded by the prsA gene. In a preferred aspect of this invention, a prsA gene coding for a feedback resistant phosphoribosyl pyrophosphate synthetase enzyme is used.

In another embodiment of this invention, as a way of conserving the PRPP serving as the co-substrate in the production of NMN from Nam, the other pathways utilizing PRPP pool within the cell are blocked. In one aspect of the present invention, the pyrE gene is mutated leading to the inactivation of the orotate phosphoribosyl transferase enzyme responsible for catalyzing the transfer of a ribosyl phosphate group from PRPP to orotate, leading to the formation of orotidine.

In another embodiment of the present invention, the microbial host cell selected for producing NMN using Nam as a feedstock and having a codon optimized exogenous gene coding for nicotinamide phosphoribosyl transferase enzyme further comprises an additional genetic modification that assures the conversion of the most of the Nam to NMN production while the other biochemical pathway for Nam utilization such as the conversion of Nam to nicotinic acid is blocked. In one aspect of this embodiment, the pncA gene is mutated so that there is a deletion of nicotinamidase enzyme function responsible for the deamidation of Nam into nicotinic acid (NA).

In another embodiment of the present invention, the microbial host cell selected for producing NMN using Nam as a feedstock and having a codon optimized exogenous gene coding for nicotinamide phosphoribosyl transferase enzyme further comprises an additional genetic modification to assure the conservation of the NMN produced within the cell using Nam as a feedstock wherein the additional genetic modification blocks other biochemical pathways that consume NMN. In one aspect of this embodiment, the ushA gene coding for an enzyme capable of the conversion of NMN to NR is deleted. In another aspect of this embodiment, the pncC gene is deleted resulting in the loss of nicotinamide-nucleotide amidohydrolase enzyme activity capable of the conversion of NMN into nicotinate mononucleotide (NAMN). In yet another aspect of this invention, the genes cgl1364, cgl1977 and cgl2835 are deleted leading to the elimination of the putative nucleosidase capable of the conversion of NMN into Nam and ribose-5 phosphate. In another aspect of this invention, the gene nadD is genetically modified so that the activity of the enzyme capable of the conversion of NMN to NAD is blocked.

In another aspect of the present embodiment, the microbial host cell selected for producing NMN using Nam as a feedstock and having a codon optimized exogenous gene coding nicotinamide phosphoribosyl transferase enzyme further comprises an additional genetic modification resulting in the knockout or impairment of a pgi gene, thereby redirecting the D-glucose-6-phosphate from glycolysis to the pentose phosphate pathway (PPP) and increasing PRPP availability. However, the deletion of pgi has been found to cause retarded growth in some microbial species in certain media. To at least partially restore normal growth, the microbial host cell may be further genetically modified to express a heterologous ZWF enzyme with a higher ability to overcome feedback inhibition relative to the native ZWF of the microbial host cell.

In non-limiting example embodiments, the transgenic ZWF enzyme may be (i) a feedback resistant mutant polypeptide comprising an amino acid sequence having at least 90%, at least 95%, at least 96%, at least 97%, at least 98%, at least 99%, or even 100% identity to the amino acid sequence as set out in SEQ ID NO: 28; (ii) a Leuconostoc mesenteroides mutant polypeptide comprising an amino acid sequence having at least 90%, at least 95%, at least 96%, at least 97%, at least 98%, at least 99%, or even 100% identity to the amino acid sequence as set out in SEQ ID NO: 30; or (iii) a polypeptide comprising an amino acid sequence having at least 90%, at least 95%, at least 96%, at least 97%, at least 98%, at least 99%, or even 100% to the Zymomonas mobilis (zmZWF) amino acid sequence as set out in SEQ ID NO: 32.

It has also been found that, in microbial species lacking native pyridine nucleotide transhydrogenases, growth may also be rescued by overcoming NADPH accumulation. This may be achieved by further genetically modifying the host cell to include one or more soluble or membrane-bound transhydrogenases. In non-limiting examples, UdhA from E. coli is a soluble transhydrogenase that is reported to favor the reduction of NAD+ by NADPH, and PntAB from E. coli is a membrane-bound transhydrogenase that is reported to favor the reduction of NADP+ by NADH.

In one representative embodiment, the recombinant ZWFs and the transhydrogenase are paired in a cgDVS expression vector.

In various embodiments of the present invention, the microbial host cell selected for producing NMN using Nam as a feedstock and having a codon optimized exogenous gene coding for nicotinamide phosphoribosyl transferase enzyme may comprise any combination of the various genetic modifications described in the foregoing paragraphs. Accordingly, a microbial host cell according to the present invention may comprise, for example, at least one, at least two, at least three, at least four, or at least five genetic modifications as described in the foregoing paragraphs.

In a further embodiment, the present invention provides a method for producing NAD using Nam as a feedstock. In one aspect of this embodiment, the microbial host cell selected for producing NMN using Nam and having a codon optimized exogenous gene coding nicotinamide phosphoribosyl transferase enzyme further comprises an additional genetic modification in nadD gene yielding an upregulated NAD(+) synthetase enzyme responsible for the conversion of NMN to NAD. In yet another aspect of this embodiment, the gene nudC is mutated so that the enzyme responsible for the conversion of NAD back to NMN is blocked.

In yet another embodiment, the present invention provides the method for producing NR using Nam as a feedstock. In one aspect of this embodiment, the microbial host cell selected for producing NMN using Nam and having a codon optimized exogenous gene coding nicotinamide phosphoribosyl transferase enzyme further comprises a functionally active dephosphorylation enzyme coded by ushA gene. In another aspect of this invention, the ushA gene is genetically modified yielding a dephosphorylation enzyme with enhanced activity for the conversion of NMN to NR. In yet another aspect of this invention, the genes cgl1364, cgl1977 and cgl2835 are deleted leading to the elimination of the purine nucleosidase responsible for the conversion of NR into NA and ribose.

Accordingly, in one aspect, the present invention provides methods of producing NMN, wherein such method comprises: feeding Nam to a culture of a genetically modified Corynebacterium glutamicum strain, where said strain comprises at least one genetic modification selected from the group consisting of: (a) heterologous expression of an nadV gene encoding a nicotinamide phosphoribosyltransferase capable of the conversion of Nam to NMN; (b) deletion or modification of one or more genes capable of the enzymatic conversion of Nam to NA; (c) deletion or modification of one or more genes capable of the enzymatic conversion of NMN to NR; (d) modification of one or more genes capable of producing PRPP; (e) modification or deletion of PRPP-requiring biochemical pathways other than the biochemical pathway for the conversion of Nam to NMN; (f) deletion or modification of the gene capable of the enzymatic conversion of NMN to NAMN; (g) deletion or modification of the gene capable of the conversion of NMN to NAD; (h) deletion or modification of one or more genes coding for an NMN nucleosidase capable of the conversion of NMN to Nam and ribose-5-phosphate; (i) incorporation of a conditionally active ribonucleotide reductase to block cell division and biomass accumulation without affecting protein synthesis and normal metabolic activity; and (j) any combinations thereof. In various embodiments, the present genetically modified strain with said at least one modification produces an increased amount of NMN compared to a strain without any of said modifications.

Therefore, in another aspect, the present invention provides methods of producing NR, wherein such method comprises: feeding Nam to a culture of a genetically modified Corynebacterium glutamicum strain, where said strain comprises at least one genetic modification selected from the group consisting of: (a) heterologous expression of an nadV gene encoding a nicotinamide phosphoribosyltransferase capable of the conversion of Nam to NMN; (b) deletion or modification of one or more genes capable of the enzymatic conversion of Nam to NA; (c) modification of one or more genes, including ushA gene, capable of the enzymatic conversion of NMN to NR; (d) modification of one or more genes capable of producing phosphoribosyl pyrophosphate (PRPP); (e) modification or deletion of PRPP-requiring biochemical pathways other than the biochemical pathway for the conversion of Nam to NMN; (f) deletion or modification of the gene capable of the enzymatic conversion of NMN to NAMN; (g) deletion or modification of the gene capable of the conversion of NMN to NAD; (h) deletion or modification of one or more genes coding for an NMN nucleosidase capable of the conversion of NMN to Nam and ribose-5-phosphate; (i) deletion or modification of one or more genes coding for a nucleosidase capable of the conversion of NR to Nam and ribose-sugar; (j) incorporation of a conditionally active ribonucleotide reductase to block cell division and biomass accumulation without affecting protein synthesis and normal metabolic activity; and (k) any combinations thereof. In various embodiments, the present genetically modified strain with said at least one modification produces an increased amount of NR compared to a strain without any of said modifications. In some embodiments, at least one of the one or more genes coding for an NMN nucleosidase capable of converting NMN to Nam and ribose-5phosphate can be the same gene(s) as at least one of the one or more genes coding a nucleosidase capable of converting NR to Nam and ribose-sugar.

Accordingly, in yet another aspect, the present invention provides methods of producing NAD, wherein such method comprises: feeding nicotinamide to a culture of a genetically modified Corynebacterium glutamicum strain, where said strain comprises at least one genetic modification selected from the group consisting of: (a) heterologous expression of an nadV gene encoding a nicotinamide phosphoribosyltransferase capable of the conversion of Nam to NMN; (b) deletion or modification of one or more genes capable of the enzymatic conversion of Nam to NA; (c) deletion or modification of one or more genes capable of the enzymatic conversion of NMN to NR; (d) modification of one or more genes capable of producing PRPP; (e) modification or deletion of PRPP-requiring biochemical pathways other than the biochemical pathway for the conversion of Nam to NMN; (f) deletion or modification of the gene capable of the enzymatic conversion of NMN to NAMN; (g) modification of the gene capable of the conversion of NMN to NAD; (h) deletion or modification of one or more genes capable of the conversion of NAD back to NMN; (i) deletion or modification of one or more genes coding for an NMN nucleosidase capable of the conversion of NMN to Nam and ribose-5-phosphate; (j) incorporation of a conditionally active ribonucleotide reductase to block cell division and biomass accumulation without affecting protein synthesis and normal metabolic activity; and (k) any combinations thereof. In various embodiments, the present genetically modified strain with said at least one modification produces an increased amount of NAD compared to a strain without any of said modifications. In some embodiments, at least one of the one or more genes coding for an NMN nucleosidase capable of converting NMN to Nam and ribose-5-phosphate can be the same gene(s) as at least one of the one or more genes coding a nucleosidase capable of converting NR to Nam and ribose-sugar.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

FIG. 1. Chemical structures of desired products nicotinamide adenine dinucleotide (NAD⁺), nicotinamide mononucleotide (NMN), and nicotinamide riboside (NR). Also shown in this figure are the chemical structure of side products namely nicotinate adenine dinucleotide (NAAD), nicotinate mononucleotide (NAMN), nicotinate riboside (NAR), as well as substrates nicotinamide (Nam) and nicotinic acid (NA). The side products are eliminated by genetic modifications according to the present invention. The structures of NA, NAR, NAMN and NAAD are different from Nam, NR, NMN, and NAD respectively by the end group C(O)OH versus C(O)NH₂.

FIG. 2. Certain genetic modifications for increased production of NR, NAD and NMN according to the present teachings. Such genetic modifications may include one or more of: (a) heterologous expression of nadV gene encoding nicotinamide phosphoribosyltransferase capable of the conversion of Nam to NMN; (b) deletion or modification of one or more genes capable of the enzymatic conversion of Nam to NA; (c) deletion or modification of one or more genes capable of the enzymatic conversion of NMN to NR; (d) modification of one or more genes capable of producing PRPP; (e) modification or deletion of PRPP requiring biochemical pathways other than the biochemical pathway for the conversion of Nam to NMN; (f) deletion or modification of the gene responsible for the enzymatic conversion of NMN to NAMN; (g) deletion or modification of the gene responsible for the conversion of NMN to NAD; and (h) deletion or modification of one or more genes coding for a promiscuous NMN nucleosidase capable of the conversion of NMN to Nam and ribose-5-phosphate.

FIG. 3. Pathway for NMN production from Nam as a feedstock in an example genetically modified microbial host cell according to the present invention.

FIG. 4. Mechanism of action of purine nucleosidase known in the art and the unexpected observation of the same enzyme functioning as nicotinamide mononucleotide nucleosidase.

FIG. 5. Method used for deleting a target gene using the suicidal plasmid pDEL. As shown in this illustration, the upstream and downstream flanking regions of a target gene to be deleted were obtained using PCR and cloned into a pDEL plasmid. C. glutamicum cells were transformed with a pDEL plasmid with upstream and downstream flanking regions of the gene targeted for deletion. After transformation, the cells were plated in a kanamycin plate to select the transformants. In the second round of screening, the transformants were plated on a sucrose containing plates to select the double recombinants with the deletion of target gene.

FIG. 6. Production of NMN in two different Corynebacterium glutamicum strains constructed according to the present invention. The strain NMN4 has the genotype of Δcgl1777-8 ΔpncA ΔpncC ΔushA ΔpyrE and the strain NR5 has the genotype of Δcgl1777-8 ΔpncA ΔpncC ΔpnuC Δcgl1364 Δcgl1977 Δcgl2835.

FIG. 7. Production of NMN in four different Corynebacterium glutamicum strains constructed according to the present invention.

FIG. 8. Pathway for enhancing PRPP production from D-glucose-6-phosphate in an example genetically modified microbial host cell according to the present invention.

FIG. 9 Growth of a number of Corynabacterium glutamicum strains transformed with ZWF and transhydrogenase genes in CGXII medium.

FIG. 10 NMN production in a number of ΔPGI Corynabacterium glutamicum strains containing indicated plasmids.

### DETAILED DESCRIPTION

The present invention relates to the construction of genetically modified host cells useful in the biological production of NMN, NR and NAD using Nam or NA as feedstock and the method of producing NMN, NR and NAD using those genetically modified host cells. In a preferred embodiment, Nam is used as the feedstock for the production of NMN. NR and NAD.

Genetically modified host cells useful in the present invention may be microbial cells such as bacterial cells, fungal cells and yeast cells. Bacterial cells useful in the present invention include, without limitation, Escherichia spp., Streptomyces spp., Zymomonas spp. Acetobacter spp., Citrobacter spp., Synechocystis spp., Rhizobium spp., Clostridium spp., Corynebacterium spp., Streptococcus spp., Xanthomonas spp. Lactobacillus spp. Lactococcus spp. Bacillus spp., Alcaligenes spp., Pseudononas spp., Aerononas spp., Azotobacter spp., Comamonas spp., Mycobacterium spp., Rhodococcus spp. Gluconobacter spp., Ralstonia spp. Acidithiobacillus spp., Microlunatus spp., Geobacter spp., Geobacillus spp., Arthrobacter spp., Flavobacterium spp. Serratia spp., Saccharopolyspora spp. Thermus spp., Stenotrophomonas spp. Chromobacterium spp., Sinorhizobium spp., Saccharopolyspora spp., Agrobacterium spp., Pantoea spp., and Vibrio natriegens. In a preferred embodiment, Corynebacterium glutamicum is used.

Yeast cells of the present disclosure include, without limitation, engineered Saccharomyces spp., Schizosaccharomyces, Hansenula, Candida, Kluyveromyces, Yarrowia, Candida boidinii, and Pichia. According to the current disclosure, a yeast as claimed herein are eukaryotic, single-celled microorganisms classified as members of the fungus kingdom. Yeasts are unicellular organisms which evolved from multicellular ancestors but with some species useful for the current disclosure being those that have the ability to develop multicellular characteristics by forming strings of connected budding cells known as pseudo hyphae or false hyphae.

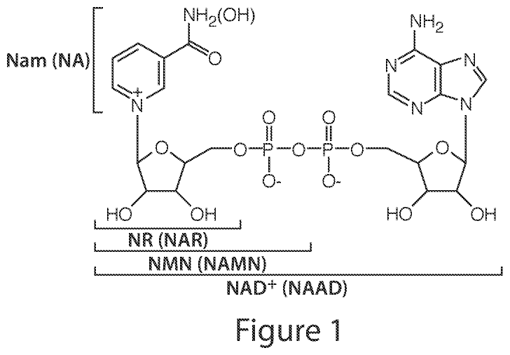

FIG. 1 shows the chemical structures of desired products NAD, NMN and NR. Also shown in this figure are the chemical structure of side products namely NAAD, NAMN, NAR, as well as substrates Nam and NA. The side products are eliminated by genetic modifications according to the present invention. The structures of NA, NAR, NAMN and NAAD are different from Nam, NR, NMN, and NAD respectively by the end group C(O)OH versus C(O)NH₂.

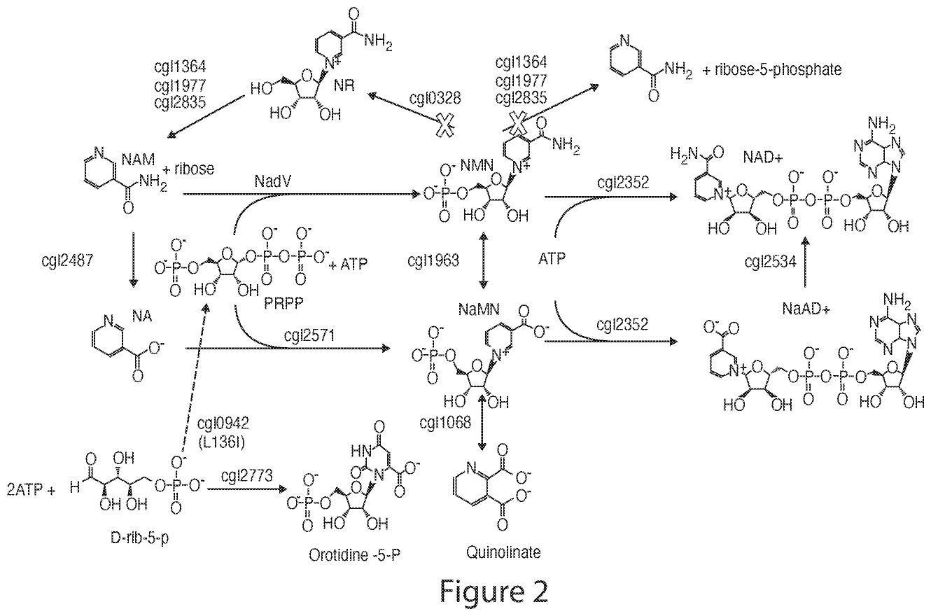

FIG. 2 shows the portions of the metabolic pathway inside a C. glutamicum cell which is relevant to the present invention. The objective of the present invention is to produce NMN, NR and NAD using Nam or NA as the feedstock while eliminating related side products such as NAR, NAMN and NAAD.

In a preferred embodiment Nam is used as the feedstock. As illustrated in FIG. 2, the metabolic pathway from Nam to NMN, NR and NAD is different from the metabolic pathway from NA to NAD, NMN and NR. The pathway from NA to NAD (via NAMN and NAAD), NMN (via NAMN), and NR (via NAMN and NMN) involves significantly more enzymatic steps than the pathway from Nam to NMN, and NR and NAD (via NMN). Therefore, Nam is considered as the preferred feedstock for producing NMN, NR, and NAD.

When Nam is used as the preferred feedstock, it is advantageous to address the conversion of Nam into NA by the deamidase enzyme coded by the pncA gene. One way to eliminate the conversion of Nam into NA is to inactivate the pncA gene. The inactivation of the pncA gene can be accomplished using a number of genetic manipulation techniques. The preferred genetic manipulation of pncA gene is to delete the nucleotide sequence from the chromosomal DNA of the microbial host cell.

Using a variety of tools available for genetic manipulations, a person skilled in the art of microbial strain construction will be able to design the metabolic pathways for producing each of the desired compounds namely NMN, NR and NDA. As illustrated in FIG. 2, the production of NMN from Nam is accomplished in a single enzymatic step within the microbial host cell. Once NMN is accumulated within the microbial host cell, the other two compounds of interest namely, NDA and NR can be obtained from NMN as a derivative product through subsequent enzymatic reactions.

Tables 1-3 lists the various genes and enzymes involved in the biochemical pathways illustrated in FIG. 2. Table 4 provides the sequence information for the genes and enzymes involved in the operation of the biochemical pathways illustrated in FIG. 2.

To provide a more direct route for the production of nicotinamide NMN, NR, and NAD, a number of genetic modifications can be made to Corynebacterium glutamicum or a related organism. In some embodiments, such genetic modifications can include heterologous expression of a gene encoding nicotinamide phosphoribosyltransferase (nadV). Nicotinamide phosphoribosyltransferase is an activity not natively found in Corynebacterium. However, it is found in a number of other bacterial and eukaryotic microorganisms. Table 2 lists certain preferred sources of such gene. In some embodiments, the genetic modifications can include deletion or modification of the gene (pncA) capable of the conversion of nicotinamide to nicotinic acid resulting in the loss or decrease in enzyme activity. In some embodiments, the genetic modifications can include modification of the prsA gene capable of the production of phosphoribosyl pyrophosphate (PRPP), a key precursor to NMN, NR, and NAD, such that higher levels of PRPP are available. Such modifications can include upregulation of gene expression and/or introduction of protein variants that lead to increased levels of enzyme activity under production conditions. Modifications to the prs gene such as those described in Marinescu et al., “Beta-nicotinamide mononucleotide (NMN) production in Escherichia coli,” Sci. Rep., 8: 12278 (2018), and Zakataeva et al., “Wild-type and feedback-resistant phosphoribosyl pyrophosphate synthetases from Bacillus amyloliquefaciens: purification, characterization, and application to increase purine nucleoside production,”Appl. Microbiol. Biotechnol., 93: 2023-33 (2012), may be used according to the present invention. In some embodiments, the modifications can include deletion or modification of the gene (pncC) responsible for the conversion of NMN to NaMN, resulting in loss or decrease in enzyme activity. In some embodiments, the modifications can include generation and incorporation of a conditionally active ribonucleotide reductase using a) temperature-sensitive (ts) mutation(s) in the protein coding sequence, b) is self-splicing intein, c) ligand-dependent gene expression, and/or d) ligand-dependent enzyme inactivation. The purpose of the modification is to block cell division and biomass accumulation while maintaining protein synthesis and metabolic activity by inhibiting deoxyribonucleotide synthesis required for DNA production and replication.

### Modified Pathway for NMN Production

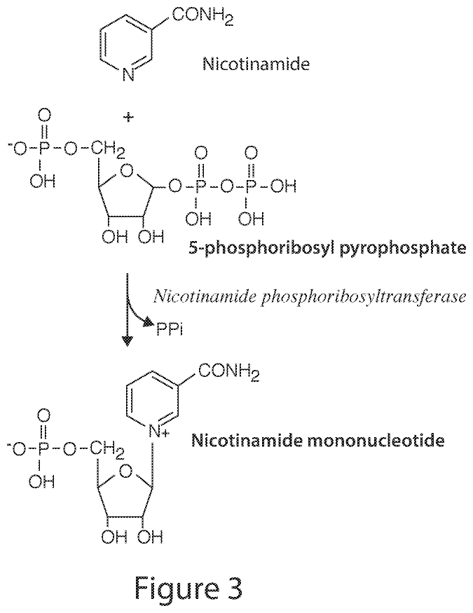

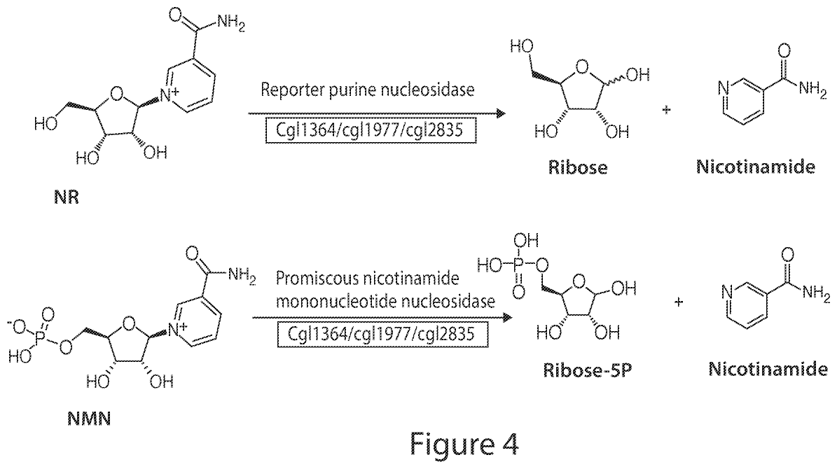

As shown in FIGS. 2 and 3, NMN production from Nam can be achieved within an engineered host cell through a single enzymatic step involving nicotinamide phosphoribosyl transferase enzyme coded by an nadV gene. In those microorganisms lacking an endogenous gene coding for a phosphoribosyl transferase enzyme such as C. glutamicum, it is necessary to introduce an exogenous gene coding for a phosphoribosyl transferase enzyme using well-known techniques in the field of genetic engineering. In a preferred aspect of the present invention, the source of nadV gene coding for nicotinamide phosphoribosyl transferase enzyme can be selected from the group consisting of Stenotrophomonas maltophilia, Chromobacterium violaceum, Deinococcus radiodurans, Synechocystis sp. PCC 6803. Pseudonocardia dioxanivorans CB1190, Shewanella oneidensis MR-1, and Ralstonia solanacearum GMI1000. In an exemplary embodiment of the present invention, an nadV gene from any one of the foregoing microbial species is isolated and introduced into a strain of Corynebacterium glutamicum using genetic engineering techniques well-known in the art. In a preferred aspect, the exogenous gene nadV is codon optimized before its transfer into a selected microbial host cell to assure optimal expression of the nicotinamide phosphoribosyl transferase enzyme in the microbial host cell.

In the phosphoribosyl transferase mediated enzyme reaction, phosphoribosyl pyrophosphate (PRPP) acts as a co-substrate and it is necessary to make sure that there is a sufficient amount of PRPP available within the cell to facilitate the phosphoribosyl transferase enzyme-mediated reaction. The pool size of PRPP within the microbial may be increased by means of enhancing the expression of the activity of a PRPP synthetase. The enhanced expression of PRPP synthetase activity can be achieved by means of expressing an exogenous gene prsA. In addition, if there is any feedback inhibition on the PrsA enzyme activity, it is preferable to use a prsA gene that encodes a feedback-resistant variant of the PrsA enzyme.

In another embodiment of this invention, as a way of conserving the PRPP pool serving as the co-substrate in the production of NMN from Nam, additional genetic modifications can be introduced to block other pathways utilizing PRPP pool within the cell. In one aspect of the present invention, the pyrE gene is mutated leading to the inactivation of the orotate phosphoribosyl transferase enzyme capable of catalyzing the transfer of a ribosyl phosphate group from PRPP to orotate, which in turn leads to the formation of orotidine.

In order to achieve the production target for NMN production, besides from ensuring that the appropriate phosphoribosyl transferase and phosphoribosyl pyrophosphate (PRPP) synthetase enzymes are present and sufficient amount of PRPP are present within the microbial cell for production of NMN from Nam, it is advantageous to block or inactivate possible NMN utilization/degradation pathways within the microbial cell.

For example, the microbial host cell selected for producing NMN using Nam as a feedstock and having a codon optimized exogenous gene coding for nicotinamide phosphoribosyl transferase enzyme can further comprise additional genetic modifications to ensure the conservation of the NMN produced within the cell, wherein the additional genetic modifications block other biochemical pathways that consume NMN. In one aspect of this embodiment, the ushA gene coding for an enzyme capable of the conversion of NMN to NR is deleted. In another aspect of this embodiment, the gene pncC is deleted resulting in the loss of nicotinamide-nucleotide amidohydrolase enzyme activity capable of the conversion of NMN into nicotinate mononucleotide (NAMN). In yet another aspect of this invention, the genes cgl1364, cgl1977 and cgl2835 are deleted leading to the elimination of a putative nucleosidase capable of the conversion of NMN into Nam and ribose-5 phosphate. In another aspect of this invention, the gene nadD is genetically modified so that the activity of the enzyme capable of the conversion of NMN to NAD is blocked.

Accordingly, in one aspect, the present invention provides methods of producing NMN, wherein such method comprises: feeding nicotinamide to a culture of a genetically modified Corynebacterium glutamicum strain, where said strain comprises at least one genetic modification selected from the group consisting of: (a) heterologous expression of a gene encoding nicotinamide phosphoribosyltransferase capable of the conversion of Nam to NMN; (b) deletion or modification of one or more genes capable of the enzymatic conversion of Nam to NA; (c) deletion or modification of one or more genes capable of the enzymatic conversion of NMN to NR; (d) modification of one or more genes capable of producing PRPP; (e) modification or deletion of PRPP-requiring biochemical pathways other than the biochemical pathway for the conversion of Nam to NMN; (f) deletion or modification of a gene capable of the enzymatic conversion of NMN to NAMN; (g) deletion or modification of a gene capable of the conversion of NMN to NAD; (h) deletion or modification of one or more genes coding for a promiscuous nucleosidase reaction capable of the conversion of NMN to Nam and ribose-5-phosphate; (i) incorporation of a conditionally active ribonucleotide reductase to block cell division and biomass accumulation without affecting protein synthesis and normal metabolic activity; and (j) any combinations thereof. In various embodiments, the present genetically modified strain with said at least one modification produces an increased amount of NMN compared to a strain without any of said modifications.

### Modified Pathway for NAD Production

In yet another embodiment, the present invention provides a method for producing NAD using Nam as a feedstock. In one aspect of this embodiment, the microbial host cell selected for producing NMN using Nam and having a codon optimized exogenous gene coding nicotinamide phosphoribosyl transferase enzyme can further comprise an additional genetic modification in the nadD gene yielding an upregulated NAD(+) synthetase enzyme capable of the conversion of NMN to NAD. In yet another aspect of this embodiment, the gene nudC is mutated so that enzyme capable of the conversion of NAD back to NMN is blocked.

Accordingly, in yet another aspect, the present invention provides methods of producing NAD, wherein such method comprises: feeding nicotinamide to a culture of a genetically modified Corynebacterium glutamicum strain, where said strain comprises at least one genetic modification selected from the group consisting of: (a) heterologous expression of a gene encoding a nicotinamide phosphoribosyltransferase capable of the conversion of Nam to NMN; (b) deletion or modification of one or more genes capable of the enzymatic conversion of Nam to NA; (c) deletion or modification of one or more genes capable of the enzymatic conversion of NMN to NR; (d) modification of one or more genes capable of producing PRPP; (e) modification or deletion of PRPP requiring biochemical pathways other than the biochemical pathway for the conversion of Nam to NMN; (f) deletion or modification of the gene capable of the enzymatic conversion of NMN to NAMN; (g) modification of the gene capable of the conversion of NMN to NAD; (h) deletion or modification of one or more genes capable of the conversion of NAD back to NMN; (i) deletion or modification of one or more genes coding for a nucleosidase capable of the conversion of NMN to Nam and ribose-5-phosphate; (j) incorporation of a conditionally active ribonucleotide reductase to block cell division and biomass accumulation without affecting protein synthesis and normal metabolic activity; and (k) any combinations thereof. In various embodiments, the present genetically modified strain with said at least one modification produces an increased amount of NMN compared to a strain without any of said modifications.

### Modified Pathway for NR Production

In yet another embodiment, the present invention provides a method for producing NR using Nam as a feedstock. In one aspect of this embodiment, the microbial host cell selected for producing NMN using Nam and having a codon optimized exogenous gene coding nicotinamide phosphoribosyl transferase enzyme can further comprise a functionally active dephosphorylation enzyme coded by a ushA gene. In another aspect of this invention, the ushA gene is genetically modified yielding a dephosphorylase enzyme with enhanced activity for the conversion of NMN to NR.

Accordingly, in another aspect, the present invention provides methods of producing NR, wherein such method comprises: feeding nicotinamide to a culture of a genetically modified Corynebacterium glutamicum strain, where said strain comprises at least one genetic modification selected from the group consisting of: (a) heterologous expression of a gene encoding a nicotinamide phosphoribosyltransferase capable of the conversion of Nam to NMN; (b) deletion or modification of one or more genes capable of the enzymatic conversion of Nam to NA; (c) modification of one or more genes capable of the enzymatic conversion of NMN to NR; (d) modification of one or more genes capable of producing PRPP; (e) modification or deletion of PRPP requiring biochemical pathways other than the biochemical pathway for the conversion of Nam to NMN; (f) deletion or modification of the gene capable of the enzymatic conversion of NMN to NAMN; (g) deletion or modification of the gene capable of the conversion of NMN to NAD; (h) deletion or modification of one or more genes coding for a nucleosidase enzyme capable of the conversion of NMN to Nam and ribose-5-phosphate; (i) deletion or modification of one or more genes coding for a nucleosidase enzyme capable of the conversion of NR to Nam and ribose-sugar, (j) incorporation of a conditionally active ribonucleotide reductase to block cell division and biomass accumulation without affecting protein synthesis and normal metabolic activity; and (k) any combinations thereof. In various embodiments, the present genetically modified strain with said at least one modification produces an increased amount of NR compared to a strain without any of said modifications.

### Modified Pathway for Increasing NMN Titer

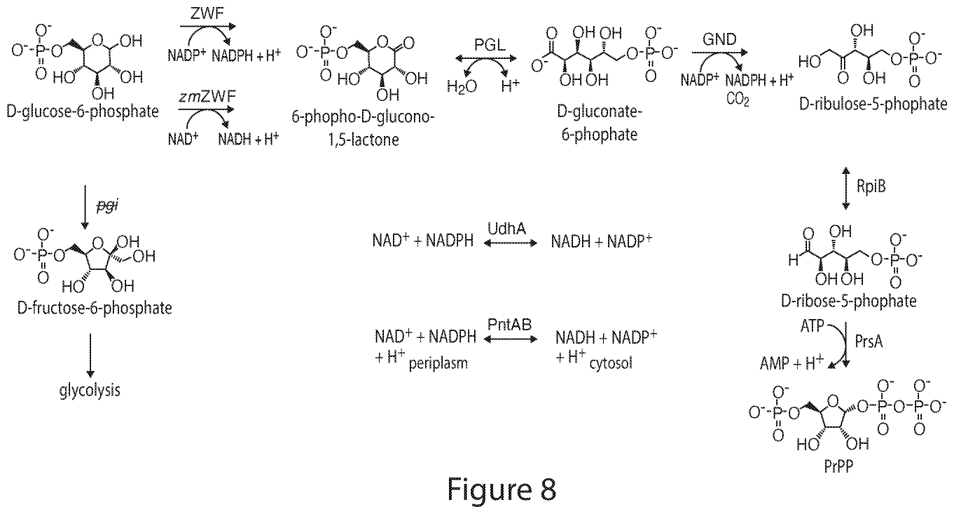

The production of NMN from NAM as catalyzed by the NadV enzyme depends at least in part upon the rate at which microbial host cells produce the PRPP co-substrate. Illustrated in FIG. 8 is the oxidative branch of the pentose phosphate pathway (“PPP”) in which D-glucose-6-phosphate is converted to D-ribulose-5-phosphate and then PRPP. A strategy to increase flux through the PPP entails knocking out the pgi gene, thereby redirecting the D-glucose-6-phosphate from glycolysis to the PPP and increasing PRPP availability. However, a problem associated with this approach in Corynabacterium gluamicum is that growth is greatly retarded on minimal media such as CGXII. Without wishing to be bound to any particular theory, it is believed that the native ZWF protein, i.e., the glucose 6-phosphate dehydrogenase enzyme catalyzing the first step of the PPP pathway, is inhibited by ATP and PEP (phosphoenolpyruvate) accumulation during growth on glucose, thereby greatly reducing carbon flux through the PPP. Moreover, assuming that an alternative ZWF can be identified that allows carbon flux to increase, a further problem emerges likely due to the accumulation of redox equivalents in the form of NADPH instead of NADH, and wild-type Corynebacterium lacks a pyridine nucleotide transhydrogenase, e.g. a soluble UdhA or a transmembrane PntAB, to address this shortcoming.

Accordingly, in one aspect, the present invention provides a recombinant Δpgi mutant of Corynebacteriun glutamicum where the expression of a transgenic ZWF enzyme in combination with a pyridine nucleotide transhydrogenase rescues growth while improving production of PRPP as reflected in higher titers of NMN. Hence, in a related aspect, the present invention provides a method of producing NMN, wherein the method comprises: feeding nicotinamide to a culture of a genetically modified Corynebacterium glutamicum strain, wherein said strain comprises genetic modifications including: (a) heterologous expression of a gene encoding nicotinamide phosphoribosyltransferase capable of the conversion of Nam to NMN; (b) deletion or modification of one or more genes capable of the enzymatic conversion of Nam to NA; (c) deletion or modification of one or more genes capable of the enzymatic conversion of NMN to NR; (d) modification of one or more genes capable of producing PRPP; (e) modification or deletion of PRPP-requiring biochemical pathways other than the biochemical pathway for the conversion of Nam to NMN; (f) deletion or modification of a gene capable of the enzymatic conversion of NMN to NAMN; (g) deletion or modification of a gene capable of the conversion of NMN to NAD; (h) deletion or modification of one or more genes coding for a promiscuous nucleosidase reaction capable of the conversion of NMN to Nam and ribose-5-phosphate; (i) deletion or modification of a gene capable of the enzymatic conversion of D-glucose-6-phosphate to D-fructose-6-phosphate; (j) incorporation of a recombinant ZWF; (k) incorporation of a recombinant pyridine nucleotide transhydrogenase, where the combination of the recombinant ZWF and the recombinant pyridine nucleotide transhydrogenase allows for compensating the growth defect associated with the deletion or modification of a gene capable of the enzymatic conversion of D-glucose-6-phosphate to D-fructose-6-phosphate; (1) incorporation of a conditionally active ribonucleotide reductase to block cell division and biomass accumulation without affecting protein synthesis and normal metabolic activity; and (m) any combinations thereof.

The production of PRPP is thereby improved as reflected in higher titers of NMN relative to a strain without such modifications.

Nicotinamide compounds (NMN, NR, NAD) produced according to the present disclosure can be utilized in any of a variety of applications, for example, exploiting their biological or therapeutic properties (e.g., controlling low-density lipoprotein cholesterol, increasing high-density lipoprotein cholesterol, etc.). For example, according to the present disclosure, nicotinamide ribose may be used in pharmaceuticals, foodstuffs, and dietary supplements, etc.

The nicotinamide mononucleotide (NMN) produced by the method disclosed in this invention could have therapeutic value in improving plasma lipid profiles, preventing stroke, providing neuroprotection with chemotherapy treatment, treating fungal infections, preventing or reducing neurodegeneration, or in prolonging health and well-being. Thus, the present invention is further directed to the nicotinamide riboside compounds obtained from the genetically modified bacterial cell described above, for treating a disease or condition associated with the nicotinamide riboside kinase pathway of NAD+ biosynthesis by administering an effective amount of a nicotinamide riboside composition.

Diseases or conditions which typically have altered levels of NAD+ or NAD+ precursors or could benefit from increased NAD+ biosynthesis by treatment with nicotinamide riboside include, but are not limited to, lipid disorders (e.g., dyslipidemia, hypercholesterolemia or hyperlipidemia), stroke, neurodegenerative diseases (e.g., Alzheimer's, Parkinsons and Multiple Sclerosis), neurotoxicity as observed with chemotherapies, Candida glabrata infection, and the general health declines associated with aging. Such diseases and conditions can be prevented or treated by diet supplementation or providing a therapeutic treatment regime with a nicotinamide riboside composition.

It will be appreciated that, the nicotinamide compounds isolated from the genetically modified bacteria of this invention can be reformulated into a final product. In some other embodiments of the disclosure, nicotinamide riboside compounds produced by genetically modified host cells as described herein are incorporated into a final product (e.g., food or feed supplement, pharmaceutical, etc.) in the context of the host cell. For example, host cells may be lyophilized, freeze dried, frozen or otherwise inactivated, and then whole cells may be incorporated into or used as the final product. The host cell may also be processed prior to incorporation in the product to increase bioavailability (e.g., via lysis).

In some embodiments of the disclosure, the produced nicotinamide riboside compounds are incorporated into a component of food or feed (e.g., a food supplement). Types of food products into which nicotinamide riboside compounds can be incorporated according to the present disclosure are not particularly limited, and include beverages such as milk, water, soft drinks, energy drinks, teas, and juices; confections such as jellies and biscuits; fat-containing foods and beverages such as dairy products; processed food products such as rice, bread, breakfast cereals, or the like. In some embodiments, the produced nicotinamide riboside compound is incorporated into a dietary supplement, such as, for example, a multivitamin.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these examples, while indicating preferred embodiments of the subject technology, are given by way of illustration only. From the above discussion and these examples, one skilled in the art can ascertain the essential characteristics of the subject technology, and without departing from the spirit and scope thereof, can make various changes and modifications of the subject technology to adapt it to various uses and conditions.

### EXAMPLES

### Example 1

### Strain Construction

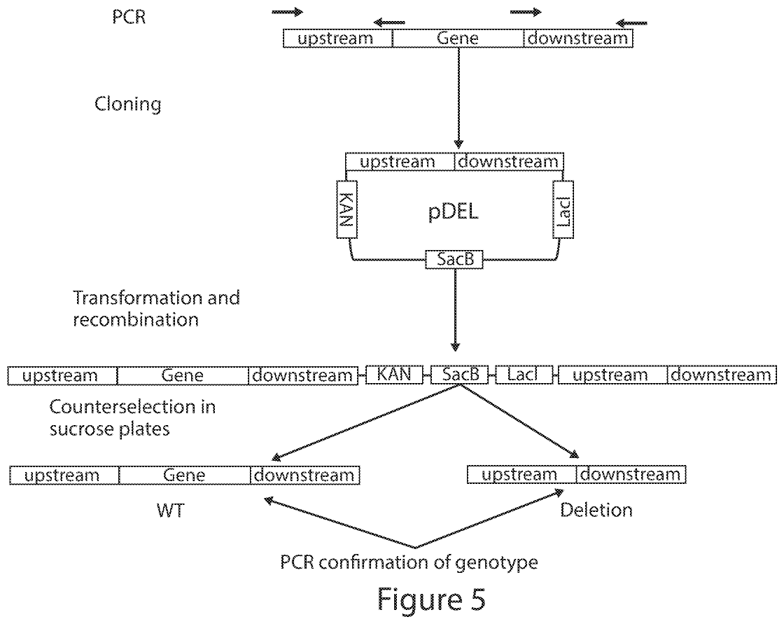

Various engineered C. glutamicum strains were constructed with one or more genetic modifications as described in Table 5. In carrying out the genetic deletions in C. glutamicum summarized in Table 5, pDEL plasmids (a suicidal plasmid which lacks an origin of replication for C. glutamicum) were used. The pDEL plasmid contained homologous regions (approximately 500-800 bps each) upstream and downstream of the gene targeted for deletion. As shown in FIG. 5, the upstream and downstream regions flanking the gene targeted for deletion were obtained using PCR and cloned into the pDEL plasmid containing an SacB gene and an antibiotic marker.

C. glutamicum cells were transformed with such plasmids and plated in CASO medium containing kanamycin (25 ug/mL) and left overnight. The resulting colonies were plated in a medium containing 6% sucrose to select for double recombinants which have target gene deletion and does not have any portion of the pDEL plasmid integrated into its chromosomal DNA.

The above engineered strains were subjected to further genetic modifications using plasmids described in Table 6 to introduce an exogenous NadV gene and a prsA gene variant/mutant.

### Example 2

### NMN Production from Nam

Strains constructed according to Example 1 were grown overnight in a BHI medium containing kanamycin. The cells were pelleted down, washed and resuspended in CGXI1 medium for 2-3 hrs for recovery as the C. glutamicum cells have a long lag phase when moved from one medium to another medium. To start the fermentation assay, cells were inoculated in a CGXII medium (Table 7) at 0.2 OD₆₀₀, at 30° C. and kept on a rotary shaker (250 rpm). The cells were induced with 0.4 mM IPTG when cell density reaches 0.6-0.8 OD₆₀₀. Nam was fed to the cell culture, and samples were collected every 24 h for up to 72 h to confirm NMN production.

Specifically, samples were obtained from the culture supernatant and centrifuged at 0.4000× G for 5 minutes. The clear supernatant was mixed with equal volume of methanol, vortexed for 30 seconds or shaken at 600 rpm on a plate shaker for 10 minutes and centrifuged at >4000× G for 5 minutes to remove any additional debris and the resultant supernatant was analyzed by HPLC method using a Phenomenex Luna 3 μm NH2 100 Å operating in HILAC mode with a maximum pressure of 400 Barr. 2 μL injections were monitored at 261 nm with standard curves generated using standards of NAM, NR, NMN, and NAD+. The aqueous buffer was 5 mM ammonium acetate pH 9.9 (A) while the organic buffer was acetonitrile (B). The HPLC column was run at 0.1 mL/min with the following gradient: 0 min-95% B, 1 min-95% B, 15 min-0% B, 20 min-0% B, 20.01 min-95% B, 30 min-95% B.

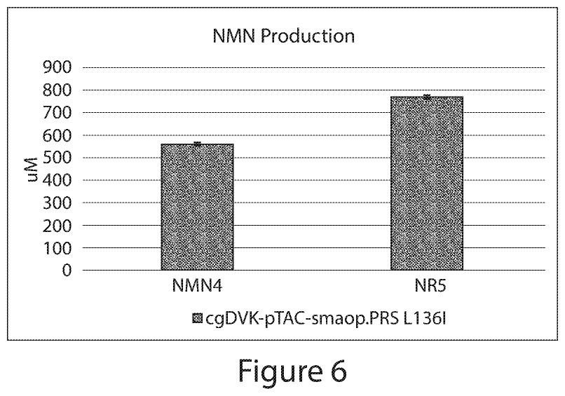

The production titers of NMN by the NMN4 and NR5 strains (both containing exogenous genes smaOP and prsA L136I), respectively, are shown in FIG. 6.

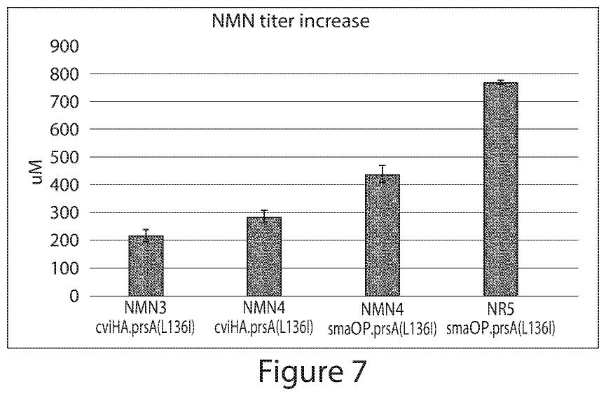

The production titers of NMN by an NMN3 strain containing exogenous genes cviHA and prsA L136I, an NMN4 strain containing exogenous genes cviHA and prsA L136I, an NMN4 strain containing exogenous genes smaOP and prsA L136I, and an NR5 strain containing exogenous genes smaOP and prsA L136I, respectively, are shown in FIG. 7.

As shown in FIG. 6 and FIG. 7, the NR5 strain characterized by the deletion of endogenous genes cgl1364 coding for purine nucleosidase iunH3, cgl1977 coding for purine nucleosidase iunH2, and cgl2835 for purine nucleosidase iunH1 appears to have the highest NMN production titers. Without wishing to be bound by any particular theory, it is believed that deletion of one or more of the foregoing genes can lead to a significant decrease in the degradation of NMN back into nicotinamide (Nam) and ribose-5P. This observation was highly surprising given the fact that the deletion of one of more of cgl1364, cgl1977, and cgl2835 was previously known only to have the effect of reducing the degradation of NR into NA and ribose.

### Example 3

### Growth Rescue and Increase in the PPP Pathway

As anticipated above, the pgi gene may be knocked out to redirect the D-glucose-6-phosphate away from glycolysis and into the PPP, thereby increasing PRPP availability. However, the Δpgi mutant of C. glutamicum is negatively affected by greatly impaired growth. Without being bound to any particular theory, it is believed that the native ZWF protein, i.e., the glucose 6-phosphate dehydrogenase enzyme catalyzing the first step of the PPP pathway, is inhibited by ATP and PEP (phosphoenolpyruvate) accumulation during growth on glucose, thereby greatly reducing carbon flux through the PPP.

Three recombinant ZWFs were screened for their ability to overcome the inhibition displayed by native C. glutamicum ZWF: (i) a feedback resistant mutant (A243T) of C. glutamicum ZWF (SEQ ID NO: 28); (ii) an engineered mutant (R46E/Q47E) of the ZWF from Leuconostoc mesenteroides (lmZWF, SEQ ID NO: 32) which has mixed use of NADH and NADPH; and (iii) the ZWF from Zymomonas mobilis (zmZWF) which favors NADH use.

Two transhydrogenases were screened for their ability to overcome NADPH accumulation: (i) UdhA from E. coli is a soluble transhydrogenase that is reported to favor the reduction of NAD+ by NADPH; and (ii) PntAB from E. coli is a membrane-bound transhydrogenase that is reported to favor the reduction of NADP+ by NADH.

The recombinant ZWFs and the transhydrogenase were paired in the cgDVS expression vector in constitutively expressed or cumate-induced configurations, as follows:

### Constitutive Expression:

(i) cgDVS.pSOD.lmZWF.pntAB (S.lmZWF.pntAB)
(ii) cgDVS.pSOD.lmZWF.udhA (S.lmZWF.udhA)
(iii) cgDVS.pSOD.zmZWF.pntAB (S.zmZWF.pntAB)
(iv) cgDVS.pSOD.zmZWF.udhA (S.zmZWF.udhA)

### Cumate-Induced Expression:

(v) cgDVS.pCT5.cgZWF*.udhA (S.pCT5.cg.udhA)

The above vectors (i)-(v) were each independently transformed into the “BASE” strain, i.e., the NMN11 strain previously transformed with the cgDVK.ptac.smaOP.prsA vector. The BASE strain was also transformed with either of two control vectors cgDVS expressing red fluorescent protein mCherry from either the min3 constitutive promoter (S.min3.mCH) or the pCT5-induced promoter (S.pCT5.mCH). The transformed strains were then grown on CGXII medium to identify instances of successful rescue from growth deficit in NMN11.

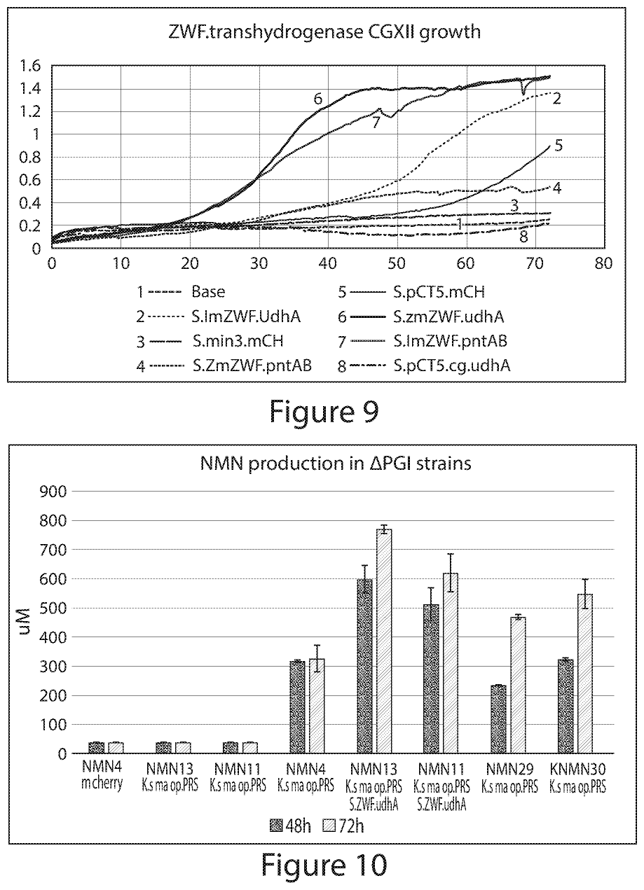

As illustrated in FIG. 9, where the time is measured in hours on the x-axis and the OD₆₀₀ is shown on the y-axis, the BASE strain, min3.mCherry and pCT5.mCherry exhibited almost zero growth, as expected. The pCT5.mCherry strain eventually emitted a signal at a wavelength of 630 nm, but still showed no major visible growth or mCherry concentration by visual examination, which would explain the later rise in emission at 630 nm as mCherry is still absorbing in the vicinity of such wavelength. The zmZWF.udhA (dark blue) and lmZWF.PntAB (yellow) strains exhibited the best rescues of the growth defect. The second-best results were obtained from lmZWF.udhA, while zmZWF.pntAB showed a modest early growth that trailed off. Initial production tests carried out in flasks revealed that strain zmZWF.udhA produced the most NMN.

The following strains were transformed with the vectors as indicated:
NMN4: Δcgl1777-8 Δcgl2487 Δcgl1963 Δcgl0328 Δcgl2773
   (1) No control: cgDVK.ptac.mcherry (mCherry)
   (2) Production control: cgDVK.ptac.smaOP.prsA (K.smaOP.PRS)
NMN13: Δcgl1777-8 Δcgl2487 Δcgl963 Δcgl0328 Δcgl0851
   (3) cgDVK.ptac.smaOP.prsA
   (4) cgDVK.ptac.smaOP.prsA+cgDVS.pSOD.zmZWF.udhA (S.ZWF.udhA)
NMN11: Δcgl1777-8 Δcgl2487 Δcgl9%3 Δcgl0328 Δcgl0851 Δcgl2773
   (5) cgDVK.ptac.smaOP.prsA
   (6) cgDVK.ptac.smaOP.prsA+cgDVS.pSOD.zmZWF.udhA

Two strains were also manufactured with the pSOD.zmZWF.udhA construct inserted into the pgi location to do away with the need for cgDVS and the spectinomycin antibiotic:
NMN29: Δcgl1777-8 Δcgl2487 Δcgl1963 Δcgl0328 Δcgl0851: pSODzmZWF.udhA
   (7) cgDVK.ptac.smaOP.prsA
NMN30: Δcgl1777-8 Δcgl2487 Δcgl1963 Δcgl0328 Δcgl0851: pSODzmZWF.udhA Δcgl2773
   (8) cgDVK.ptac.smaOP.prsA

The amount of NMN produced by each strain was assessed after 48 hours and 72 hours of cell culture growth, respectively. The results are illustrated in FIG. 10.

TABLE 1
Genes and enzymes used in the present invention

| | Corynebacterium | | |
|---|---|---|---|
| Gene | glutamicum ATCC 13032 | | EC |
| name | NCBI gene symbol | Name of the enzyme | Number |
| pncA | NCgl2401 | Nicotinamidase/ | 3.5.1.19 |
| | | pyrazinamidase | |
| prs | NCgl0905 | Ribose-phosphate | 2.7.6.1 |
| | | pyrophosphokinase or | |
| | | PRPP synthase | |
| pncB | NCgl2431 | Nicotinate | 6.3.4.21 |
| | | phosphoribosyltransferase | |
| pncC | NCgl1888 | Nicotinamide-nucleotide | 3.5.1.42 |
| | | amidohydrolase | |
| nadD | NCgl2270 | Nicotinate-nucleotide | 2.7.7.18 |
| | | adenylyltransferase | |
| nadE | NCgl2446 | NH(3)-dependent | 6.3.1.5 |
| | | NAD(+) synthetase | |
| nudC | NCgl0744 | NADH pyrophosphatase | 3.6.1.22 |
| cgl0328 | NCgl0322 | 5-nucleotidase | 3.1.3.5 |
| cgl1364 | NCgl1309 | Nicotinamide riboside | 3.2.2.1 |
| | | hydrolase | |
| nadV | Not present in | Nicotinamide | 2.4.2.12 |
| | Corynebacterium | phosphoribosyltransferase | |
| | glutamicum ATCC 13032. | | |

TABLE 2
Preferred sources and genes for nadV coding for nicotinamide
phosphoribosyltransferase activity

| Source of nadV gene | NCBI gene ID |
|---|---|
| Chromobacterium violaceum ATCC 12472 | CV_RS00140 |
| Deinococcus radiodurans R1 | DR_0294 |
| Synechocystis sp. PCC 6803 | slr0788 |
| Pseudonocardia dioxanivorans CB1190 | Psed_2146 |
| Shewanella oneidensis MR-1 | SO_1981 |
| Ralstonia solanacearum GMI1000 | RSp0836 |
| Stenotrophomonas maltophilia | B2FI76 |
| Haemophilus ducreyi | NAMPT gene |

TABLE 3
Additional genes and proteins used in the present invention

| | Gene | | |
|---|---|---|---|
| | Accession | | |
| Gene | Number: | Protein | Details |
| cviHA | AAQ57714 | cviNadVha | Codon harmonized variant of the |
| | | | nicotinamide |
| | | | phosphoribosyltransferase (NadV) |
| | | | from Chromobacterium violaceum |
| smaOP | SQG65624 | smaNadVop | Codon optimized variant of the |
| | | | nicotinamide |
| | | | phosphoribosyltransferase (NadV) |
| | | | from Stenotrophomonas maltophilia |
| prsA | | PrsA (PRS) | Codon optimized variant of the |
| | | | PRPP synthase from |
| | | | Corynebacterium glutamicum |
| | | | ATCC 13032 |
| prsA | | PrsA L136I | Feedback resistant mutant of PrsA |
| L136I | | (PRS | |
| | | L136I) | |
| zmZWF | | zmZWF | Codon optimized variant of the |
| | | | glucose-6-phosphate dehydrogenase |
| | | | from Zymomonas mobilis strain |
| | | | ATCC 10988, Zmob_0908 |
| lmZWF | | lmZWF | Codon optimized mutant |
| | | R46E/Q47E | (R46E/Q47E) of the glucose-6- |
| | | | phosphate dehydrogenase from |
| | | | Leuconoston mesenteroides |
| cgZWF* | | cgZWF | Codon optimized mutant (A243T) |
| | | A243T | of the of the glucose-6-phosphate |
| | | | dehydrogenase from |
| | | | Corynebacterium glutamicum |
| udhA | | udbA | Soluble Pyridine Nucleotide |
| | | | Transhydrogenase from Escherichia |
| | | | coli |
| pntAB | | pntA.B | Membrane-bound Pyridine |
| | | | Nucleotide Transhydrogenase from |
| | | | Escherichia coli |

TABLE 4
Sequence Information

| | |
|---|---|
| SEQ ID No. 1 | PRT; Chromobacterium violaceum; nicotinamide |
| | phosphoribosyltransferase-cviNadVha (cviHA) |
| SEQ ID No. 2 | PRT; Stenotrophomonas maltophilia; nicotinamide |
| | phosphoribosyltransferase-smaNadVop (smaOP) |
| SEQ ID No. 3 | PRT; codon-optimized PRPP synthase variant, CYL77_RS04805-prsA |
| | (PRS) |
| SEQ ID No. 4 | PRT; feedback resistant PRPP synthase mutant, prsA L136I (PRS |
| | L136I) |
| SEQ ID No. 5 | DNA; nicotinamidase pncA (CGL_RS12350, ncgl2401, cgl2487 |
| SEQ ID No. 6 | PRT; nicotinamidase pncA (CGL_RS12350, ncgl2401, cgl2487 |
| SEQ ID No. 7 | DNA; Nicotinamide-nucleotide amidohydrolase pncC (CGL_RS09770, |
| | ncgl1888, cgl1963) |
| SEQ ID No. 8 | PRT; Nicotinamide-nucleotide amidohydrolase pncC (CGL_RS09770, |
| | ncgl1888, cgl1963) |
| SEQ ID No. 9 | DNA; restriction modification system; RM = cgl1777 |
| | (CYL77_RS08985) |
| SEQ ID No. 10 | PRT; restriction modification system; RM = cgl1777 (CYL77_RS08985) |
| SEQ ID No. 11 | DNA; restriction modification system; RM = cgl1778 |
| | (CYL77_RS08990) |
| SEQ ID No. 12 | PRT; restriction modification system; RM = cgl1778 (CYL77_RS08990) |
| SEQ ID No. 13 | DNA; orotate phosphoribosyltransferase pyrE; CGL_RS13820, |
| | ncgl2676 |
| SEQ ID No. 14 | PRT; orotate phosphoribosyltransferase pyrE; CGL_RS13820, |
| | ncgl2676, cgl2773 |
| SEQ ID No. 15 | DNA; 5′-nucleotidase ushA; cgl0328-CGL_RS01710, ncgl0322, |
| | cg0397 |
| SEQ ID No. 16 | PRT; 5′-nucleotidase ushA; cgl0328-CGL_RS01710, ncgl0322, cg0397 |
| SEQ ID No. 17 | DNA; nicotinamide ribose transporter pnuC; ncgl0063, CGL_RS00355, |
| | cgl0064 |
| SEQ ID No. 18 | PRT; nicotinamide ribose transporter pnuC; ncgl0063, CGL_RS00355, |
| | cgl0064 |
| SEQ ID No. 19 | DNA; purine nucleosidase iunH3; cgl1364 (CGL_RS06810, ncgl1309, |
| | cgl543, iunH3) |
| SEQ ID No. 20 | PRT; purine nucleosidase iunH3; cgl1364 (CGL_RS06810, ncgl1309, |
| | cg1543, iunH3) |
| SEQ ID No. 21 | DNA; purine nucleosidase iunH2; cgl1977 (cg2168, iunH2, |
| | CYL77_RS09970) |
| SEQ ID No. 22 | PRT; purine nucleosidase iunH2; cgl1977 (cg2168, iunH2, |
| | CYL77_RS09970) |
| SEQ ID No. 23 | DNA; purine nucleosidase iunH1; cgl2835 (cg3137, iunH1, |
| | CYL77_RS14340) |
| SEQ ID No. 24 | PRT; purine nucleosidase iunH1; cgl2835 (cg3137, iunH1, |
| | CYL77_RS14340) |
| SEQ ID No. 25 | DNA; glucose-6P isomerase pgi; cgl0851 (ncgl0817) |
| SEQ ID No. 26 | PRT; glucose-6P isomerase PGI; cgl0851 (ncgl0817) |
| SEQ ID No. 27 | DNA; codon-optimized mutant (A243T) of the of the glucose-6- |
| | phosphate dehydrogenase from Corynebacterium glutamicum; cgZWF |
| SEQ ID No. 28 | PRT; codon-optimized mutant (A243T) of the of the glucose-6- |
| | phosphate dehydrogenase from Corynebacterium glutamicum; cgZWF |
| SEQ ID No. 29 | DNA; glucose-6-phosphate dehydrogenase of |
| | Leuconoston.mesenteroides, codon optimized mutant (R46E/Q47E) of |
| | the gene having accession number M64446.1; lmZWF |
| SEQ ID No. 30 | RNA; glucose-6-phosphate dehydrogenase of |
| | Leuconoston.mesenteroides, codon optimized mutant (R46E/Q47E); |
| | lmZWF |
| SEQ ID No. 31 | DNA; codon-optimized variant of the glu6-phosphate dehydrogenase |
| | from Zymomonas mobilis strain ATCC 10988, Zmob_0908 |
| SEQ ID No. 32 | PRT; codon-optimized variant of the glu6-phosphate dehydrogenase |
| | from Zymomonas mobilis strain ATCC 10988, NCBI-ProteinID: |
| | AEH62743.1 |
| SEQ ID No. 33 | DNA; soluble Pyridine Nucleotide Transhydrogenase from Escherichia |
| | coli; udhA NP_418397.2, EG11428 |
| SEQ ID No. 34 | PRT; soluble Pyridine Nucleotide Transhydrogenase from Escherichia |
| | coli; udhA AAC76944 |
| SEQ ID No. 35 | DNA; “A” subunit of Membrane bound Pyridine Nucleotide |
| | Transhydrogenase (pntA) from Escherichia coli MG1655; ECK1598, |
| | variant g1342a, NP_416120.1 |
| SEQ ID No. 36 | PRT; “A” subunit of Membrane bound Pyridine Nucleotide |
| | Transhydrogenase (pntB) from Escherichia coli; P07001.2 A434T |
| | variant |
| SEQ ID No. 37 | DNA; “B” subunit of Membrane bound Pyridine Nucleotide |
| | Transhydrogenase (pntB) from Escherichia coli MG1655; ECK1597, |
| | NP_416119.1 |
| SEQ ID No. 38 | PRT; “B” subunit of Membrane bound Pyridine Nucleotide |
| | Transhydrogenase (pntB) from Escherichia coli; P0AB69.1 |

TABLE 5
Strains constructed according to the present invention

| Strain name | Genotype/Description of genetic modification |
|---|---|
| ATCC 13032 | Corynebacterium glutamicum ATCC 13032 |
| Cgl1 | ATCC 13032 Δcgl1777 Δcgl1778-Deletion of restriction |
| | modification (RM) system |
| NMN1 | Cgl1 Δcgl2487-Deletion of nicotinamidase (pncA) to |
| | prevent conversion of NA to NAM |
| NMN2 | NMN1 Δcgl1963-Deletion of nicotinamide-nucleoside |
| | amidase (pncC) to prevent conversion of NMN to NaMN. |
| NMN3 | NMN2 Δcgl0328-Deletion of 5′-nucleotidase (ushA) to |
| | prevent conversion of NMN to NR. |
| NMN4 | NMN3 Δcgl2773-Deletion of orotate |
| | phosphoribosyltransferase (pyrE) to prevent conversion |
| | of PRPP to Orotidine-5-P; Uracil auxotroph. |
| | ΔRM ΔpncA ΔpncC ΔushA ΔpyrE |
| NR1 | NMN2 Δcgl1364-deletion of purine nucleosidase (iunH3) |
| | to prevent conversion of NR to NAM. |
| NR2 | NR1 Δcgl0064-Deletion of nicotinamide ribose |
| | transporter (pnuC) to inhibit NR uptake |
| NR3 | NR2 Δcgl2835-Deletion of purine nucleosidase (iunH1) |
| | to prevent conversion of NR to NAM |
| NR5 | NR3 Δcgl1977-deletion of purine nucleosidase (iunH2) to |
| | prevent conversion of NR to NAM |
| | ΔRM (9, 11) ΔpncA (5) ΔpncC (7) ΔpnuC (17) Δcgl1364 |
| | Δcgl1977 Δcgl2835 (19, 21, 23) |
| NMN11 | NMN4 Δcgl0851-Deletion of glucose-6P isomerase |
| | (PGI) to push carbon flux towards PPP |
| NMN13 | NMN3 Δcgl0851-Deletion of glucose-6P isomerase |
| | (PGI) to push carbon flux towards PPP |
| NMN16 | NR5 Δcgl0328-Deletion of 5′-nucleotidase (ushA) to |
| | prevent conversion of NMN to NR |
| NMN17 | NMN16 Δcgl2773-Deletion of orotate |
| | phosphoribosyltransferase (pyrE) to prevent conversion |
| | of PRPP to Orotidine-5-P; Uracil auxotroph |
| NMN29 | NMN11 Δcgl0851:zmZWF.udhA-Insertion of |
| | zmZWF.udhA into PGI locus to increase carbon |
| | flux towards PPP |
| NMN30 | NMN13 Δcgl0851:zmZWF.udhA-Insertion of |
| | zmZWF.udhA into PGI locus to increase carbon |
| | flux towards PPP |

TABLE 6
Plasmids for introducing exogenous genes

| Plasmid Code | Plasmid Name | Description |
|---|---|---|
| cgDVK | | E. coli/C. glut. Shuttle vector |
| | | ColE1 ori/pHM1519 ori KanR |
| cviHA.prsA(L136I) | cgDVK.pTAC.cviHA.prsA(L136I) | cgDVK constitutive LacI |
| | | production, IPTG induced |
| | | expression of cviNadVha and |
| | | PrsA(L136I) |
| smaOP.prsA(L136I) | cgDVK.pTAC.smaOP.prsA(L136I) | cgDVK constitutive LacI |
| | | production, IPTG induced |
| | | expression of smaNadVop and |
| | | PrsA(L136I) |
| cgDVS | | E. coli/C. glut shuttle vector |
| | | pBL1/ori SpecR |
| zmZWF.udhA | cgDVS.pSOD.zmZWF.udhA | cgDVS constitutive pSOD |
| | | expression of zmZWF.udhA |
| zmZWF.pntAB | cgDVS.pSOD.zmZWF.pntAB | cgDVS constitutive pSOD |
| | | expression of zmZWF.pntAB |
| lmZWF.udhA | cgDVS.pDOS.lmZWF.udbA | cgDVS constitutive pSOD |
| | | expression of lmZWF.udhA |
| lmZWF.pntAB | cgDVS.pSOD.lmZWF.pntAB | cgDVS constitutive pSOD |
| | | expression of lmZWF.pntAB |
| cgZWF*.udhA | cgDVS.pCT5.cgZWF*.udhA | cgDVS constitutive CymR, |
| | | Cumate induced expression of |
| | | cgZWF*.udhA |

TABLE 7
Chemical Composition of CGXII
Growth medium

| Component | Conc./L |
|---|---|
| NH₄SO₄ | 20 g |
| KH₂PO₄ | 1 g |
| K₂HPO₄ | 1 g |
| Urea (300 gl) | 5 g |
| MgSO₄ * 7H₂O (1M) | 0.25 g |
| CaCl₂*2H₂O (1M) | 10 mg |
| CG TE 50x | See paper |
| PCA (50 gl in 57% etoh) | 30 mg |
| Biotin (10 gl) | 0.2 mg |
| Thiamine HCl (20 gl) | 1 mg |
| MOPS | 42 g |
| Glucose (50%) | 30 g |

Sequences of Interest
SEQ ID NO: 1; PRT; Chromobacterium violaceum; nicotinamide
phosphoribosyltransferase-cviNadVha (cviHA)
MTAPKAAQDKNQLVPFNLADFYKTGHPAMYPRETTRLVANFTPRSAKYAQVLPQLF
DDKVVWFGLQGFIQEYLIDLFNREFFQRPKADAVRRYQRRMDTALGAGAVDGGRLE
ALHDLGHLPLEIRSLPEGARVDIKVPPVTFSNTHPDFPWVATYFETLFSCESWKPSTVA
TIAFEFRKLLSYFAALTGAPQDFVAWQGHDFSMRGMSGVHDAMRCGAGHLLSFTGT
DTIPALDYLEDHYGADAERELVGGSIPASEHSVMALRILLTQQRLARMPAHQGLDDK
ALRRLAEREVVREFVTRDYPAGMVSIVSDTFDFWNVLTVIARELKDDIQARRPDALGN
AKVVFRPDSGDPVRILAGYRDDELQFDDAGNCTARDDGRPVSAAERKGAVECLWDIF
GGTVTERGYRVLDSHVGLIYGDSITLPRARDILLRLAEKGYASCNVVFGIGSFVYGMN
SRDTFGYALKAVYAEVAGEAVDIYKDPATDDGTKKSARGLLRVEEENGRYALYQQQ
TPAEAEGGALRPVFRDGELLVKQTLAEIRQRLQASWTCPEAGSIVWNA
SEQ ID NO: 2; PRT; Stenotrophomonas maltophilia; nicotinamide
phosphoribosyltransferase-smaNadVop (smaOP)
MHYLDNLLLNTDSYKASHWLQYPPGTDATFFYVESRGGLHDRTVFFGLQAILKDALA
RPVTHADIDDAAAVFAAHGEPFNEAGWRDIVDRLGGHLPVRIRAVPEGSVVPTHQAL
MTIESTDPAAFWVPSYLETLLLRVWYPVTVATISWHARQTIAAFLQQTSDDPQGQLPF
KLHDFGARGVSSLESAALGGAAHLVNFLGTDTVSALCLARAHYHAPMAGYSIPAAEH
STITSWGREREVDAYRNMLRQFGKPGSIVAVVSDSYDIYRAISEHWGTTLRDDVIASG
ATLVIRPDSGDPVEVVAESLRRLDEAFGHAINGKGYRVLNHVRVIQGDGINPDTIRAIL
QRITDDGYAADNVAFGMGGALLQRLDRDTQKFALKCSAARVEGEWIDVYKDPVTDA
GKASKRGRMRLLRRLDDGSLHTVPLPANGDDTLPDGFEDAMVTVWENGHLLYDQRL
DDIRTRAAVGH
SEQ ID NO: 3; PRT; Codon-optimized PRPP synthase variant,
CYL77_RS04805-prsA (PRS)
MTAHWKQNQKNLMLFSGRAHPELAEAVAKELDVNVTPMTARDFANGEIYVRFEESV
RGSDCFVLQSHTQPLNKWLMEQLLMIDALKRGSAKRITAILPFYPYARQDKKHRGREP
ISARLIADLMLTAGADRIVSVDLHTDQIQGFFDGPVDHMHAMPILTDHIKENYNLDNIC
VVSPDAGRVKVAEKWANTLGDAPMAFVHKTRSTEVANQVVANRVVGDVDGKDCV
LLDDMIDTGGTIAGAVGVLKKAGAKSVVIACTHGVFSDPARERLSACGABEVITTDTL
PQSTEGWSNLTVLSIAPLLARTINEIFENGSVTTLFEGEA
SEQ ID NO: 4; PRT; Feedback resistant PRPP synthase mutant,
prsA L136I (PRS L136I)
MTAHWKQNQKNLMLFSGRAHPELAEAVAKELDVNVTPMTARDFANGEIYVRFEESV
RGSDCFVLQSHTQPLNKWLMEQLLMIDALKRGSAKRITAILPFYPYARQDKKHRGREP
ISARLIADLMLTAGADRIVSVDIHTDQIQGFFDGPVDHMHAMPILTDHIKENYNLDNIC
VVSPDAGRVKVAEKWANTLGDAPMAFVHKTRSTEVANQVVANRVVGDVDGKDCV
LLDDMIDTGGTIAGAVGVLKKAGAKSVVIACTHGVFSDPARERLSACGAEEVITTDTL
PQSTEGWSNLTVLSIAPLLARTINEIFENGSVTTLFEGEA
SEQ ID NO.: 5; DNA; Nicotinamidase pncA (CGL_RS12350,
ncgl2401, cgl2487)
ATGGCACGCGCACTCATTCTGGTTGATGTTCAAAAAGACTTCTGCCCCGGTGGCAG
CCTAGCCACCGAACGAGGCGATGAAGTGGCGGGAAAAATCGGTGCCTATCAGCTG
TCCCACGGCTCAGAGTACGACGTCGTTGTGGCGACCCAAGATTGGCACATCGATC
CAGGCGAGCACTTTTCAGAAACCCCAGACTTTAAAAACTCCTGGCCAATCCACTGC
GTCGCGGATTCCGATGGTGCCGCCATGCATGACCGCATCAACACCGATTCAATCG
ATGAGTTCTTCCGCAAAGGCCATTACACCGCGGCGTATTCCGGGTTCGAGGGAACT
GCAGTCAGTGAAGAACTCCTCATGTCTCCATGGCTGAAGAACAAGGGAGTCACTG
ATGTAGACATCGTAGGGATCGCTACGGATCACTGCGTTCGAGCCACAGCACTTGA
TGCTCTCAAGGAGGGCTTCAACGTCTCCATTTTGACGTCGATGTGTTCTGCGGTGG
ATTTCCATGCGGGAGACCACGCTTTGGAGGAACTACATGAAGCCGGGGCGATTCT
GATTTAA
SEQ ID No.: 6; PRT; Nicotinamidase pncA (CGL_RS12350,
ncgl2401, cgl2487)
MARALILVDVQKDFCPGGSLATERGDEVAGKIGAYQLSHGSEYDVVVATQDWHIDP
GEHFSETPDFKNSWPIHCVADSDGAAMHDRINTDSIDEFFRKGHYTAAYSGFEGTAVS
EELLMSPWLKNKGVTDVDIVGIATDHCVRATALDALKEGFNVSILTSMCSAVDFHAG
DHALEELHEAGAILI
SEQ ID NO: 7; DNA; Nicotinamide-nucleotide amidohydrolase
pncC (CGL_RS09770, ncgl1888, cgl1963)
ATGTCGGAGAATCTGGCGGGGCGAGTGGTGGAGCTGTTGAAATCGCGCGGTGAAA
CGCTGGCGTTTTGTGAATCCCTCACCGCCGGCCTTGCCAGTGCGACGATCGCAGAG
ATCCCCGGCGCCTCAGTGGTACTTAAAGGCGGGCTGGTCACCTATGCCACCGAGCT
TAAGGTTGCGCTTGCCGGTGTGCCGCAGGAGCTTATCGACGCGCACGGCGTTGTTT
CCCCGCAGTGCGCCCGTGCGATGGCAACGGGGGCCGCACACAGATGCCAGGCAGA
TTGGGCGGTTTCGCTCACGGGCGTTGCTGGCCCCAGCAAACAAGATGGTCATCCG
GTGGGGGAAGTGTGGATCGGAGTGGCTGGTCCTGCGCATTTTGGGGCGTCGGGAA
CAATTGACGCGTATCGTGCGTTTGAAAGTGAACAACAGGTAATATTGGCTGAATT
GGGACGGCATCATATTAGAGAGTCTGCTGTGCAGCAAAGCTTTCGCCTGCTGATTG
ACCATATTGAGTCGCAGTGA
SEQ ID NO: 8; PRT; Nicotinamide-nucleotide amidohydrolase
pncC (CGL_RS09770, ncgl1888, cgl1963)
MSENLAGRVVELLKSRGETLAFCESLTAGLASATIAEIPGASVVLKGGLVTYATELKV
ALAGVPQELIDAHGVVSPQCARAMATGAAHRCQADWAVSLTGVAGPSKQDGHPVG
EVWIGVAGPAHFGASGTIDAYRAFESEQQVILAELGRHHIRESAVQQSFRLLIDHIESQ
SEQ ID No.: 9; DNA; Restriction modification system;
RM = cgl1777 (CYL77_RS08985)
ATGAAGCCCACCGTTAATGTTGTGTTCAATGCGCATCACCCCAAAGATACGCAGCC
GTTGGATAAGTTCTTCGATAAAGAACTTAAAGACACACATCATCTCGATATAACG
GTGGGTTATATCAGTGAGAAATCACTACAATATTTGCTTCTTATTGCAGGCACTCA
CCCCGACCTCACCATCACACTCACCTGTGGAATGCACGCTCGTGAAGGCATGACTG
CTGCCCAACTGCATCATGCGCGAGTGCTCCATGACTACTTAAGCGACCATGATCGA
GGCGGGGTGTTCGTTATTCCCCGATTGCGTTATCACGGCAAAATCTATCTTTTCCA
CAAGAACCAGCACACAGATCCTATTGCTTATATCGGTAGCGCTAACCTCTCAGCCA
TCGTTCCTGGGTACACCTCTACATTCGAGACCGGCGTCATCTTAGACCCCGCACCT
GAAGATCTCGTGCTTCATCTCAACCGTGATGTCGTACCCCTATGTGTCCCCATTGA
CACCGCGCATGTCCCCATCATTAAAGATCAAGAATCCCCGATGAAGCACGTCGCT
GAAGCAACAGCTGTGTCCACCTCTGATGTTGTTGCCATCATGTCCAGCCCATTTAC
TTATAGTTTTGACCTTAAACTCAAAGCCACTGCCAGCAGCAACCTCAATGCTCATA
ACTCAGGCGGTGGCGCGCGCAAACAGAAAAACGGTAGCTTCCTTGCACGCAATTG
GTATGAGGGCGAAATCATTGTCGGTGTCGAGACAACAAGACTCCCAGGTTACCCA
CAAAACAAATCCGAATTCACTGCGGTCACTGATGACGGCTGGTCATTTGTTTGCAA
AATCAGCGGAGGAAACGGAAAGAACCTACGCAGCAAAGGTGACCTGTCCATCCTC
GGTACGTGGTTAAAGTCTCGATTCATTGAACAAGGTGCCCTGGAATACGGCGAGG
ATGCCACCCAAGAAAACATCGACCGTTTTGGGAGAACACATATGACCATGCGCTA
TCACCCAGATTTCGATGTGTGGTCATTCGATCTCAGCCAAACCCCGAAGCCTTCGA
CACAGATTGGGCAGGATTAA
SEQ ID NO: 10; PRT; Restriction modification system;
RM = cgl1777 (CYL77_RS08985)
MKPTVNVVFNAHHPKDTQPLDKFFDKELKDTHHLDITVGYISEKSLQYLLLIAGTHPD
LTITLTCGMHAREGMTAAQLHHARVLHDYLSDHDRGGVFVIPRLRYHGKIYLFHKNQ
HTDPIAYIGSANLSAIVPGYTSTFETGVILDPAPEDLVLHLNRDVVPLCVPIDTAHVPIIK
DQESPMKHVAEATAVSTSDVVAIMSSPFTYSFDLKLKATASSNLNAHNSGGGARKQK
NGSFLARNWYEGEIIVGVETTRLPGYPQNKSEFTAVTDDGWSFVCKISGGNGKNLRSK
GDLSILGTWLKSRFIEQGALEYGEDATQENIDRFGRTHMTMRYHPDFDVWSFDLSQTP
KPSTQIGQD
SEQ ID NO: 11; DNA; Restriction modification system;
RM = cgl1778 (CYL77_RS08990)
RTCACCTCAATAACTACATCACGAGCTTGAGTGATAACGCTGATCTCCGTGAAAAA
GTCACCGCAACCGTAGACGCTTTCCGCCATACCGTCATGGATGACTTCGACTACAT
CAGTGATCAACAAGTCCTGCTTTATGGCGATGTCCAAAGCGGTAAAACCTCACAC
ATGCTGGGAATTATCGCAGATTGCCTCGACAGTACGTTTCACACCATTGTTATTCT
GACCTCGCCTAACACACGGCTCGTGCAACAAACATACGACCGTGTTGCCCAAGCA
TTTCCAGATACTTTGGTGTGCGACCGTGACGGATACAATGATTTCCGTGCGAATCA
AAAGAGCCTCACCCCGCGAAAATCTATCGTAGTCGTCGGAAAAATACCTGCAGTT
CTTGGTAATTGGTTACGCGTCTTTAACGACAGTGGCGCACTTTCTGGACACCCTGT
ACTCATTATTGATGACGAAGCAGATGCGACAAGTCTCAACACCAAAGTAAATCAG
TCTGATGTTTCGACCATTAACCACCAGCTCACTAGCATAAGAGACCTTGCCACAGG
ATGCATCTACCTTCAGGTCACAGGTACACCTCAAGCGGTGCTTCTTCAAAGCGACG
ATAGCAACTGGGCAGCGGAACATGTGCTTCACTTCGCACCTGGTGAGAGCTACAT
CGGTGGTCAACTTTTCTTTTCTGAGCTCAACAACCCTTATCTACGACTTTTCGCTAA
TACCCAATTTGACGAGGATTCTCGCTTCAGCGACGCCATTTACACCTATCTCTTAA
CCGCAGCACTGTTCAAACTTCGCGGTGAAAGCTTGTGTACCATGCTCATTCACCCC
AGCCACACTGCATCCAGTCATAGAGACTTCGCGCAAGAAGCCCGCCTCCAACTCA
CTTTCGCCTTCGAGCGATTCTATGAACCAATGATTCAGCACAATTTCCAACGTGCT
TATGAACAGCTCGCACAAACTGACAGCAACCTGCCACCCTTGAGAAAAATTCTTA
ACATTCTTGGTGGCATGGAAGATGACTTCTCCATCCACATCGTCAATAGCGACAAC
CCGACTGTTGAGGAAGATTGGGCTGATGGTTATAACATTATTGTCGGTGGCAACTC
GCTTGGGCGCGGTTTAACATTCAACAACTTGCAAACCGTTTTCTACGTGCGCGAAT
CCAAGCGACCACAAGCAGACACCCTGTGGCAGCACGCCCGCATGTTTGGCTACAA
ACGCCACAAAGACACCATGCGTGTGTTCATGCCGGCCACTATTGCTCAAACCTTCC
AAGAGGTCTATCTCGGCAACGAAGCTATTAAAAATCAGCTCGATCATGGCACGCA
TATCAACGACATTCGGGTCATTTTAGGTGATGGCGTCGCACCTACTCGTGCCAATG
TTCTCGACAAACGCAAAGTTGGAAACCTCAGCGGTGGCGTCAACTACTTTGCCGCT
GATCCTAGAATCAAGAATGTCGAAGCACTCGACAAAAAACTCTTGGCCTACTTAG
ACAAGCACGGTGAGGACTCCACCATCGGTATGCGCGCGATAATCACCATTCTCAA
CGCCTTTACTGTAGACCCCAACGATCTCGACCTCGCGACCTTCAAGGCTGCGCTCC
TTGACTTTGAACGCAACCAACCTCATCTCACAGCACGTATGGTGCTGCGAACAAAC
CGCAAAGTCAATCAGGGTACAGGCGCCCTGCTCTCCCCTACTGATCAAGCTCTCAG
CCGTGCAGAAGTCGCACACCCATTATTGATCCTATACCGCATTGAAGGTGTTAACG
ATGCTGCTGCGCAACGAGGTGAACCTACGTGGTCAAGCGACCCTATCTGGGTGCC
TAATATTAAACTCCCTGGTCAACGTCAATTCTGGTGCGTAGACGGCTAA
SEQ ID NO: 12; PRT; Restriction modification system;
RM = cgl1778 (CYL77_RS08990)
MSHHTHLNNYITSLSDNADLREKVTATVDAFRHTVMDDFDYISDQQVLLYGDVQSG
KTSHMLGIIADCLDSTFHTIVILTSPNTRLVQQTYDRVAQAFPDTLVCDRDGYNDFRA
NQKSLTPRKSIVVVGKIPAVLGNWLRVENDSGALSGHPVLIIDDEADATSLNTKVNQS
DVSTINHQLTSIRDLATGCIYLQVTGTPQAVLLQSDDSNWAAEHVLHFAPGESYIGGQ
LFFSELNNPYLRLFANTQFDEDSRFSDAIYTYLLTAALFKLRGESLCTMLIHPSHTASSH
RDFAQEARLQLTFAFERFYEPMIQHNFQRAYEQLAQTDSNLPPLRKILNILGGMEDDES
IHIVNSDNPTVEEDWADGYNIIVGGNSLGRGLTFNNLQTVFYVRESKRPQADTLWQH
ARMFGYKRHKDTMRVFMPATIAQTFQEVYLGNEAIKNQLDHGTHINDIRVILGDGVA
PTRANVLDKRKVGNLSGGVNYFAADPRIKNVEALDKKLLAYLDKHGEDSTIGMRAII
TILNAFTVDPNDLDLATFKAALLDFERNQPHLTARMVLRTNRKVNQGTGALLSPTDQ
ALSRAEVAHPLLILYRIEGVNDAAAQRGEPTWSSDPIWVPNIKLPGQRQFWCVDG
SEQ ID NO: 13; DNA; Orotate phosphoribosyltransferase pyrE;
CGL_RS13820, ncgl2676, cgl2773
ATGTCATCTAATTCCATTAACGCAGAAGCGCGCGCTGAGCTTGCTGAACTGATCAA
AGAGCTAGCTGTCGTCCACGGTGAAGTCACCTTGTCTTCGGGCAAGAAGGCTGATT
ACTACATCGATGTCCGTCGTGCCACCTTGCACGCGCGCGCATCTCGCCTGATCGGT
CAGCTGCTGCGCGAAGCCACCGCTGACTGGGACTATGACGCAGTTGGCGGCCTGA
CCTTGGGCGCTGACCCGGTTGCCACCGCCATCATGCACGCCGACGGCCGCGATATC
AACGCGTTTGTGGTGCGCAAGGAGGCCAAGAAGCACGGCATGCAGCGTCGCATTG
AGGGCCCTGACCTGACGGGCAAGAAGGTGCTCGTGGTGGAAGATACCACCACCAC
CGGAAATTCCCCTCTGACAGCTGTTGCCGCGTTGCGTGAAGCTGGCATTGAGGTTG
TGGGCGTTGCCACCGTGGTCGATCGCGCAACCGGTGCAGATGAGGTTATCGCAGC
GGAAGGCCTTCCTTACCGCAGCTTGCTGGGACTTTCTGATCTTGGACTCAACTAA
SEQ ID NO: 14; PRT; Orotate phosphoribosyltransferase pyrE;
CGL_RS13820, ncgl2676, cgl2773
MSSNSINAEARAELAELIKELAVVHGEVTLSSGKKADYYIDVRRATLHARASRLIGQL
LREATADWDYDAVGGLTLGADPVATAIMHADGRDINAFVVRKEAKKHGMQRRIEGP
DLTGKKVLVVEDTTTTGNSPLTAVAALREAGIEVVGVATVVDRATGADEVIAAEGLP
YRSLLGLSDLGLN
SEQ ID NO: 15; DNA; 5′-nucleotidase ushA; cgl0328-CGL_RS01710,
ncgl0322, cg0397
ATGAAGAGGCTTTCCCGTGCAGCCCTCGCAGTGGTCGCCACCACCGCAGTTAGCTT
CAGCGCACTCGCAGTTCCAGCTTTCGCAGACGAAGCAAGCAATGTTGAGCTCAAC
ATCCTCGGTGTCACCGACTTCCACGGACACATCGAGCAGAAGGCTGTTAAAGATG
ATAAGGGAGTAATCACCGGTTACTCAGAAATGGGTGCCAGTGGCGTTGCCTGCTA
CGTCGACGCTGAACGCGCGGACAACCCAAACACCCGCTTCATCACCGTTGGTGAC
AACATTGGTGGATCCCCATTCGTGTCCTCCATCCTGAAGGATGAGCCAACCTTGCA
AGCCCTCAGCGCCATCGGTGTTGACGCATCCGCACTGGGCAATCACGAATTCGAC
CAGGGCTACTCAGACCTGGTGAACCGCGTTTCCCTCGACGGCTCCGGCAGCGCAA
AGTTCCCATACCTCGGCGCAAACGTTGAAGGTGGCACCCCAGCACCTGCAAAGTC
TGAAATCATCGAGATGGACGGCGTCAAGATCGCTTACGTCGGCGCAGTAACCGAG
GAGACCGCAACCTTGGTCTCCCCAGCAGGCATCGAAGGCATCACCTTCACCGGCG
ACATCGACGCTATCAACGCAGAAGCAGATCGCGTCATTGAGGCAGGCGAAGCAGA
CGTAGTCATCGCATTGATCCACGCTGAAGCCGCTCCAACCGATCTATTCTCCAACA
ACGTTGACGTTGTATTCTCCGGACACACCCACTTCGACTACGTTGCTGAAGGCGAA
GCACGTGGCGACAAGCAGCCACTCGTTGTCATCCAGGGCCACGAATACGGCAAGG
TCATCTCCGACGTGGAGATCTCCTACGACCGCGAAGCAGGCAAGATCACCAACAT
TGAGGCGAAGAATGTCTCTGCTACTGACGTTGTGGAAAACTGTGAGACTCCAAAC
ACAGCAGTCGACGCAATCGTTGCAGCTGCTGTTGAGGCCGCTGAAGAAGCAGGTA
ATGAAGTTGTTGCAACCATTGACAACGGCTTCTACCGTGGGGCGGATGAAGAGGG
TACGACCGGCTCCAACCGTGGTGTTGAGTCTTCCCTGAGCAACCTCATCGCAGAAG
CTGGACTGTGGGCAGTCAACGACGCGACCATCCTGAACGCTGACATCGGCATCAT
GAACGCAGGCGGCGTGCGTGCGGACCTCGAAGCAGGCGAAGTTACCTTCGCAGAT
GCATACGCAACCCAGAACTTCTCCAACACCTACGGCGTACGTGAAGTGTCTGGTG
CGCAGTTCAAAGAAGCACTGGAACAGCAGTGGAAGGAAACCGGCGACCGCCCAC
GTCTGGCATTGGGACTGTCCAGCAACGTCCAGTACTCCTACGACGAGACCCGCGA
ATACGGCGACCGCATCACCCACATCACCTTCAACGGTGAGCCAATGGATATGAAG
GAGACCTACCGCGTCACAGGATCATCCTTCCTGCTCGCAGGTGGCGACTCCTTCAC
TGCATTCGCTGAAGGCGGCCCAATCGCTGAAACCGGCATGGTTGACATTGACCTGT
TCAACAACTACATCGCAGCTCACCCAGATGCACCAATTCGTGCAAATCAGAGCTC
AGTAGGCATCGCCCTTTCCGGCCCGGCAGTTGCAGAAGACGGAACTTTGGTCCCTG
GTGAAGAGCTGACCGTCGATCTTTCTTCCCTCTCCTACACCGGACCTGAAGCTAAG
CCAACCACCGTTGAGGTGACCGTTGGTACTGAGAAGAAGACTGCGGACGTCGATA
ACACCATCGTTCCTCAGTTTGACAGCACCGGCAAGGCAACTGTCACCCTGACTGTT
CCTGAGGGAGCTACCTCTGTCAAGATCGCAACTGACAATGGCACTACCTTTGAACT
GCCAGTAACCGTAAACGGTGAAGGCAACAATGATGACGATGATGATAAGGAGCA
GCAGTCCTCCGGATCCTCCGACGCCGGTTCCCTTGTAGCAGTTCTCGGTGTTCTTG
GAGCACTCGGTGGCCTGGTGGCGTTCTTCCTGAACTCTGCGCAGGGCGCACCATTC
TTGGCTCAGCTTCAGGCTATGTTTGCGCAGTTCATGTAA
SEQ ID NO: 16; PRT; 5′-nucleotidase ushA; cgl0328-CGL_RS01710,
ncgl0322, cg0397
MKRLSRAALAVVATTAVSFSALAVPAFADEASNVELNILGVTDFHGHIEQKAVKDDK
GVITGYSEMGASGVACYVDAERADNPNTRFITVGDNIGGSPFVSSILKDEPTLQALSAI
GVDASALGNHEFDQGYSDLVNRVSLDGSGSAKFPYLGANVEGGTPAPAKSEIIEMDG
VKIAYVGAVTEETATLVSPAGIEGITFTGDIDAINAEADRVIEAGEADVVIALIHAEAAP
TDLFSNNVDVVFSGHTHFDYVAEGEARGDKQPLVVIQGHEYGKVISDVEISYDREAG
KITNIEAKNVSATDVVENCETPNTAVDAIVAAAVEAAEEAGNEVVATIDNGFYRGAD
EEGTTGSNRGVESSLSNLIAEAGLWAVNDATILNADIGIMNAGGVRADLEAGEVTFAD
AYATQNFSNTYGVREVSGAQFKEALEQQWKETGDRPRLALGLSSNVQYSYDETREY
GDRITHITFNGEPMDMKETYRVTGSSFLLAGGDSFTAFAEGGPIAETGMVDIDLFNNYI
AAHPDAPIRANQSSVGIALSGPAVAEDGTLVPGEELTVDLSSLSYTGPEAKPTTVEVTV
GTEKKTADVDNTIVPQFDSTGKATVTLTVPEGATSVKIATDNGTTFELPVTVNGEGNN
DDDDDKEQQSSGSSDAGSLVAVLGVLGALGGLVAFFLNSAQGAPFLAQLQAMFAQF
M
SEQ ID NO: 17; DNA; Nicotinamide ribose transporter pnuC;
ncgl0063, CGL_RS00355, cgl0064
ATGAATCCTATAACCGAATTATTAGACGCAACACTATGGATCGGCGGAGTTCCGA
TTCTGTGGCGCGAAATCATCGGCAACGTTTTCGGATTATTTAGCGCGTGGGCAGGA
ATGCGACGCATCGTGTGGGCATGGCCCATCGGCATCATAGGCAACGCGCTGCTGT
TCACAGTATTTATGGGCGGCCTTTTCCACACTCCACAAAACCTCGATCTCTACGGC
CAAGCGGGTCGCCAGATCATGTTCATCATCGTCAGTGGTTATGGCTGGTACCAATG
GTCGGCCGCAAAACGTCGCGCACTCACCCCAGAAAATGCAGTAGCAGTGGTTCCT
CGCTGGGCAAGCACCAAAGAACGCGCCGGCATTGTGATTGCGGCGGTTGTGGGAA
CACTCAGCTTTGCCTGGATTTTCCAAGCACTCGGCTCCTGGGGGCCATGGGCCGAC
GCGTGGATTTTCGTCGGCTCAATCCTGGCTACCTACGGAATGGCTCGCGGATGGAC
AGAGTTCTGGCTGATCTGGATCGCCGTCGACATAGTTGGCGTTCCTCTACTTTTGA
CTGCTGGCTACTACCCATCCGCGGTGCTTTACCTGGTGTACGGTGCGTTTGTCAGC
TGGGGATTTGTCGTGTGGCTGCGGGTGCAAAAAGCAGACAAGGCTCGTGCGCTGG
AAGCTCAGGAGTCTGTGACAGTCTGA
SEQ ID NO: 18; PRT; Nicotinamide ribose transporter pnuC;
ncgl0063, CGL_RS00355, cgl0064
MNPITELLDATLWIGGVPILWREIIGNVFGLFSAWAGMRRIVWAWPIGIIGNALLFTVF
MGGLFHTPQNLDLYGQAGRQIMFIIVSGYGWYQWSAAKRRALTPENAVAVVPRWAS
TKERAGIVIAAVVGTLSFAWIFQALGSWGPWADAWIFVGSILATYGMARGWTEFWLI
WIAVDIVGVPLLLTAGYYPSAVLYLVYGAFVSWGFVVWLRVQKADKARALEAQESV
TV
SEQ ID NO: 19; DNA; Purine nucleosidase iunH3; cgl1364
(CGL_RS06810, ncgl1309, cgl543, iunH3)
ATGACCACCAAGATCATCCTCGACTGCGATCCAGGACACGACGACGCTGTAGCCA
TGCTGCTCGCAGCCGGCAGCCCAGAAATTGAACTGCTTGGAATCACCACGGTCGG
CGGCAACCAGACCTTGGACAAGGTCACCCACAATACGCAGGTCGTAGCCACCATC
GCTGATATCAATGCGCCCATCTACCGCGGTGTCACCCGACCATTGGTGCGCCCCGT
TGAGGTAGCCGAAGATATCCACGGCGATACCGGCATGGAAATCCACAAGTACGAA
CTGCCTGAACCAACCAAGCAGGTAGAAGACACCCACGCGGTGGATTTCATCATCG
ATACCATCATGAATAACGAGCCCGGCAGCGTAGCGCTGGTTCCCACCGGACCACT
GACCAACATCGCGCTGGCAGTCCGGAAAGAACCACGCATCGCCGAGCGAGTCAAG
GAAGTTGTCCTCATGGGGGGGGCTACCACGTAGGAAACTGGACCGCCGTAGCTG
AATTCAACATCAAGATCGACCCCGAAGCAGCCCACATCGTATTCAACGAAAAGTG
GCCACTGACTATGGTCGGCCTCGACCTTACCCACCAGGCGCTCGCAACACCTGAG
ATCGAAGCCAAGTTCAACGAGCTGGGCACCGACGTCGCCGACTTCGTCGTCGCGC
TTTTCGACGCTTTCCGCAAGAATTACCAGGACGCACAGGGTTTTGATAACCCACCA
GTACACGACCCTTGTGCTGTTGCATACCTTGTTGACCCAACCGTATTCACCACCCG
CAAAGCACCACTCGATGTGGAGCTGTACGGCGCACTCACCACAGGCATGACCGTT
GCTGATTTCCGCGCACCGGCTCCAGCAGATTGCACCACCCAAGTAGCTGTTGACCT
GGACTTTGATAAATTCTGGAACATGGTGATCGATGCAGTAAAGCGCATCGGATAG
SEQ ID NO: 20; PRT; Purine nucleosidase iunH3; cgl1364
(CGL_RS06810, ncgl1309, cg1543, iunH3)
MTTKIILDCDPGHDDAVAMLLAAGSPEIELLGITTVGGNQTLDKVTHNTQVVATIADI
NAPIYRGVTRPLVRPVEVAEDIHGDTGMEIHKYELPEPTKQVEDTHAVDFIIDTIMNNE
PGSVALVPTGPLTNIALAVRKEPRIAERVKEVVLMGGGYHVGNWTAVAEFNIKIDPEA
AHIVFNEKWPLTMVGLDLTHQALATPEIEAKFNELGTDVADFVVALFDAFRKNYQDA
QGFDNPPVHDPCAVAYLVDPTVFTTRKAPLDVELYGALTTGMTVADFRAPAPADCTT
QVAVDLDFDKFWNMVIDAVKRIG
SEQ ID NO: 21; DNA; Purine nucleosidase iunH2; cgl1977
(cg2168, iunH2, CYL77_RS09970)
ATGAGCAAAAAAGCCATCCTTGATATCGACACCGGCATCGATGATGCCCTCGCAC
TTGCCTACGCACTGGGCTCACCTGAACTAGAGCTCATTGGTGTCACCACCACCTAC
GGTAACGTGCTACTCGAAACCGGTGCAGTCAATGACCTGGCACTGCTTGATCTGTT
CGGTGCACCAGAAGTACCTGTGTACTTGGGTGAGCCACACGCACAGACCAAGGAT
GGCTTTGAAGTTCTTGAGATCTCCGCGTTCATTCACGGACAAAACGGCATCGGCGA
AGTCGAGCTGCCAGCAAGCGAGTCAAAGGCACTCCCCGGCGCAGTGGATTTCCTC
ATTGATTCCGTCAACACCCACGGCGATGACCTGGTGATCATCGCAACTGGTCCCAT
GACCAACCTGTCTGCGGCAATCGCAAAGGATCCAAGCTTTGCTTCCAAGGCTCAC
GTGGTCATCATGGGTGGCGCCTTGACTGTCCCAGGCAACGTCAGCACATGGGCAG
AAGCAAACATCAACCAGGACCCAGATGCAGCAAACGATCTGTTCCGTTCCGGTGC
AGATGTCACCATGATCGGTCTTGATGTCACCCTGCAGACCCTTCTTACCAAGAAGC
ACACTGCGCAGTGGCGCGAACTGGGCACTCCAGCTGCTATCGCACTGGCCGACAT
GACTGATTACTACATCAAGGCATATGAGACCACCGCACCACACCTGGGCGGTTGC
GGCCTGCACGACCCACTGGCAGTAGGCGTTGCAGTGGACCCAAGCCTGGTCACTT
TGCTCCCCATCAACCTCAAGGTAGACATTGAGGGCGAGACCCGTGGACGCACCAT
TGGCGATGAAGTCCGCCTCAACGATCCAGTGCGCACCTCCCGCGCAGCTGTCGCC
GTAGACGTGGATCGTTTCCTTTCTGAATTCATGACCCGCATCGGCCGAGTCGCAGC
ACAGCAGTAA
SEQ ID NO: 22; PRT; Purine nucleosidase iunH2; cgl1977
(cg2168, iunH2, CYL77_RS09970)
MSKKAILDIDTGIDDALALAYALGSPELELIGVTTTYGNVLLETGAVNDLALLDLFGAP
EVPVYLGEPHAQTKDGFEVLEISAFIHGQNGIGEVELPASESKALPGAVDFLIDSVNTH
GDDLVIIATGPMTNLSAAIAKDPSFASKAHVVIMGGALTVPGNVSTWAEANINQDPDA
ANDLFRSGADVTMIGLDVTLQTLLTKKHTAQWRELGTPAAIALADMTDYYIKAYETT
APHLGGCGLHDPLAVGVAVDPSLVTLLPINLKVDIEGETRGRTIGDEVRLNDPVRTSR
AAVAVDVDRFLSEFMTRIGRVAAQQ
SEQ ID No.: 23; DNA; Purine nucleosidase iunH1; cgl2835
(cg3137, iunH1, CYL77_RS14340)
ATGATTCCTGTTCTCATCGACTGCGACACCGGCATCGACGACGCC
CTCGCCCTGATCTACCTGGTTGCTTTGCATAAACGTGGTGAAATCCAACTTTTTGG
AGCAACGACCACCGCAGGAAATGTTGATGTGAAACAAACCGCCTCAATACCAGGT
GGGTGTTGGATCAGTGTGGATTAGCGGACATCCCGGTCCTCGCAGGACAACCTGA
ACCAAAGCACGTGCCGCTAGTGACTACTCCAGAAACACACGGCGACCATGGCCTT
GGTTATATAAACCCAGGTCACGTCGAAATTCCAGAAGGTGACTGGAAGCAGCTGT
GGAAAGAACACCTCAGTAACCCAGAAACTAAGCTGATTGTCACCGGGCCCGCCAC
CAACCTTGCGGAATTCGGGCCAGTGGAAAACGTCACGCTGATGGGTGGCACCTAC
CTTTATCCAGGCAACACCACTCCAACGGCAGAATGGAATACCTGGGTTGATCCAC
ACGGAGCTAAAGAAGCATTCGCGGCAGCCCAAAAGCCCATTACGGTGTGTTCCTT
GGGCGTGACCGAGCAGTTTACGCTGAACCCGGACATCCTTTCTACACTTATCAACA
CGCTTGGCAGCCAACCCATCGCAGAGCATTTACCTGAGATGCTGCGCTTTTACTTT
GAATTTCACGAAGTGCAGGGCGAAGGTTACCTTGCTCAAATTCATGACCTGCTGAC
CTGCATGATTGCCTTGGATAAAATCCCATTTTCAGGCCGTGAAGTAACCGTGGACG
TGGAGGCTGATTCGCCCTTGATGCGTGGCACCACTGTTGCAGATATTCGCGGACAT
TGGGGCAAGCCAGCTAACGCATTTCTTGTGGAAACCGCAGACATTGAGGCCGCCC
ACGCGGAACTTCTAAGAGCAGTGGAATGA
SEQ ID No.: 24; PRT; Purine nucleosidase iunH1; cgl2835
(cg3137, iunH1, CYL77_RS14340)
MIPVLIDCDTGIDDALALIYLVALHKRGEIQLFGATTTAGNVDVKQTAINTRWVLDQC
GLADIPVLAGQPEPKHVPLVTTPETHGDHGLGYINPGHVEIPEGDWKQLWKEHLSNPE
TKLIVTGPATNLAEFGPVENVTLMGGTYLYPGNTTPTAEWNTWVDPHGAKEAFAAA
QKPITVCSLGVTEQFTLNPDILSTLINTLGSQPIAEHLPEMLRFYFEFHEVQGEGYLAQI
HDLLTCMIALDKIPFSGREVTVDVEADSPLMRGTTVADIRGHWGKPANAFLVETADIE
AAHAELLRAVE
SEQ ID No. 25; DNA; glucose-6P isomerase pgi; cgl0851
(ncgl0817)
ATGGCGGACATTTCGACCACCCAGGTTTGGCAAGACCTGACCGATCATTACTCAA
ACTTCCAGGCAACCACTCTGCGTGAACTTTTCAAGGAAGAAAACCGCGCCGAGAA
GTACACCTTCTCCGCGGCTGGCCTCCACGTCGACCTGTCGAAGAATCTGCTTGACG
ACGCCACCCTCACCAAGCTCCTTGCACTGACCGAAGAATCTGGCCTTCGCGAACGC
ATTGACGCGATGTTTGCCGGTGAACACCTCAACAACACCGAAGACCGCGCTGTCC
TCCACACCGCGCTGCGCCTTCCTGCCGAAGCTGATCTGTCAGTAGATGGCCAAGAT
GTTGCTGCTGATGTCCACGAAGTTTTGGGACGCATGCGTGACTTCGCTACTGCGCT
GCGCTCAGGCAACTGGTTGGGACACACCGGCCACACGATCAAGAAGATCGTCAAC
ATTGGTATCGGTGGCTCTGACCTCGGACCAGCCATGGCTACGAAGGCTCTGCGTGC
ATACGCGACCGCTGGTATCTCAGCAGAATTCGTCTCCAACGTCGACCCAGCAGAC
CTCGTTTCTGTGTTGGAAGACCTCGATGCAGAATCCACATTGTTCGTGATCGCTTC
GAAAACTTTCACCACCCAGGAGACGCTGTCCAACGCTCGTGCAGCTCGTGCTTGGC
TGGTAGAGAAGCTCGGTGAAGAGGCTGTCGCGAAGCACTTCGTCGCAGTGTCCAC
CAATGCTGAAAAGGTCGCAGAGTTCGGTATCGACACGGACAACATGTTCGGCTTC
TGGGACTGGGTCGGAGGTCGTTACTCCGTGGACTCCGCAGTTGGTCTTTCCCTCAT
GGCAGTGATCGGCCCTCGCGACTTCATGCGTTTCCTCGGTGGATTCCACGCGATGG
ATGAACACTTCCGCACCACCAAGTTCGAAGAGAACGTTCCAATCTTGATGGCTCTG
CTCGGTGTCTGGTACTCCGATTTCTATGGTGCAGAAACCCACGCTGTCCTACCTTA
TTCCGAGGATCTCAGCCGTTTTGCTGCTTACCTCCAGCAGCTGACCATGGAATCAA
ATGGCAAGTCAGTCCACCGCGACGGCTCCCCTGTTTCCACTGGCACTGGCGAAATT
TACTGGGGTGAGCCTGGCACAAATGGCCAGCACGCTTTCTTCCAGCTGATCCACCA
GGGCACTCGCCTTGTTCCAGCTGATTTCATTGGTTTCGCTCGTCCAAAGCAGGATC
TTCCTGCCGGTGAGCGCACCATGCATGACCTTTTGATGAGCAACTTCTTCGCACAG
ACCAAGGTTTTGGCTTTCGGTAAGAACGCTGAAGAGATCGCTGCGGAAGGTGTCG
CACCTGAGCTGGTCAACCACAAGGTCATGCCAGGTAATCGCCCAACCACCACCAT
TTTGGCGGAGGAACTTACCCCTTCTATTCTCGGTGCGTTGATCGCTTTGTACGAAC
ACATCGTGATGGTTCAGGGCGTGATTTGGGACATCAACTCCTTCGACCAATGGGGT
GTTGAACTGGGCAAACAGCAGGCAAATGACCTCGCTCCGGCTGTCTCTGGTGAAG
AGGATGTTGACTCGGGAGATTCTTCCACTGATTCACTGATTAAGTGGTACCGCGCA
AATAGGTAG
SEQ ID No. 26; PRT; glucose-6P isomerase PGI; cgl0851
(ncgl0817)
MADISTTQVWQDLTDHYSNFQATTLRELFKEENRAEKYTFSAAGLHVDLSKNLLDDA
TLTKLLALTEESGLRERIDAMFAGEHLNNTEDRAVLHTALRLPAEADLSVDGQDVAA
DVHEVLGRMRDFATALRSGNWLGHTGHTIKKIVNIGIGGSDLGPAMATKALRAYATA
GISAEFVSNVDPADLVSVLEDLDAESTLFVIASKTFTTQETLSNARAARAWLVEKLGEE
AVAKHFVAVSTNAEKVAEFGIDTDNMFGFWDWVGGRYSVDSAVGLSLMAVIGPRDF
MRFLGGFHAMDEHFRTTKFEENVPILMALLGVWYSDFYGAETHAVLPYSEDLSRFAA
YLQQLTMESNGKSVHRDGSPVSTGTGEIYWGEPGINGQHAFFQLIHQGTRLVPADFIG
FARPKQDLPAGERTMHDLLMSNFFAQTKVLAFGKNABEIAAEGVAPELVNHKVMPG
NRPTTTILAEELTPSILGALIALYEHIVMVQGVIWDINSFDQWGVELGKQQANDLAPAV
SGEEDVDSGDSSTDSLIKWYRANR
SEQ ID No. 27; DNA; cgZWF codon-optimized mutant (A243T)
of the of the glucose-6-phosphate dehydrogenase from
Corynebacterium glutamicum (cg1778, Cgl1576, NCgl1514)
ATGAGTACCAACACCACCCCGTCAAGCTGGACAAATCCATTGCGCGACCCCCAGG
ATAAGCGCTTGCCCCGCATCGCAGGACCCTCCGGCATGGTCATTTTTGGGGTGACC
GGCGATCTGGCACGCAAGAAACTGCTACCAGCCATCTATGACTTGGCAAATCGCG
GCTTACTGCCACCTGGCTTCTCTCTCGTGGGCTATGGTCGCCGTGAATGGTCTAAG
GAGGACTTCGAAAAGTACGTTCGTGATGCAGCGTCCGCGGGAGCCCGAACGGAAT
TTCGTGAAAACGTCTGGGAACGCCTTGCAGAAGGCATGGAATTTGTCCGCGGAAA
TTTTGATGATGACGCCGCATTCGACAACTTGGCGGCGACGCTGAAGCGCATCGAT
AAGACGAGAGGCACTGCTGGTAACTGGGCGTACTATCTGTCCATCCCACCGGACT
CCTTTACGGCGGTGTGCCACCAGCTAGAGCGTTCCGGCATGGCTGAGTCCACCGA
AGAGGCATGGCGCCGAGTGATCATTGAAAAGCCATTCGGGCACAACCTGGAATCG
GCACACGAGCTCAACCAACTGGTCAACGCCGTTTTCCCGGAGTCATCAGTGTTTAG
AATCGATCACTACCTGGGTAAAGAAACCGTGCAGAATATCCTCGCGCTGCGATTC
GCAAATCAACTTTTTGAACCCCTTTGGAACAGCAACTATGTCGATCACGTCCAAAT
TACCATGACTGAAGATATTGGCTTGGGAGGACGCGCGGGTTATTATGATGGAATC
GGAGCAGCGCGCGACGTCATCCAGAATCACCTCATTCAGCTGTTGGCGCTGGTAG
CGATGGAGGAACCCATTAGCTTTGTGCCTGCTCAGCTGCAAGCAGAAAAGATCAA
AGTTCTGAGCGCTACCAAACCTTGTTACCCTCTGGATAAGACCTCAGCTCGCGGTC
AATATGCTGCTGGCTGGCAAGGATCTGAGCTGGTCAAGGGCCTTCGTGAAGAGGA
CGGTTTCAACCCCGAGAGCACCACGGAAACCTTCGCCGCATGTACCCTTGAAATC
ACAAGTCGCCGCTGGGCCGGCGTCCCATTCTACCTGCGTACTGGCAAGAGACTCG
GCCGACGAGTTACAGAGATCGCTGTTGTGTTTAAAGATGCTCCCCACCAGCCGTTT
GATGGAGACATGACCGTTTCCCTTGGCCAAAATGCGATCGTAATTCGCGTACAACC
AGACGAGGGTGTTCTTATCCGCTTTGGTTCCAAGGTGCCCGGTTCCGCTATGGAGG
TTCGTGACGTTAATATGGACTTCAGCTATAGCGAATCCTTCACCGAAGAGTCACCT
GAAGCATACGAACGCCTGATCCTGGATGCCCTCCTGGACGAGTCCAGCTTGTTTCC
AACCAACGAGGAAGTGGAACTGTCTTGGAAAATCCTGGACCCAATTCTGGAAGCT
TGGGATGCCGATGGCGAACCGGAGGACTACCCAGCTGGGACCTGGGGGCCAAAAT
CGGCGGATGAGATGTTATCCCGTAACGGCCACACATGGCGCCGACCTTGA
SEQ ID No. 28; DNA; cgZWF mutant (A243T) of the of the
glucose-6-phosphate dehydrogenase from Corynebacterium
glutamicum (cgl778, Cgl1576, NCgl1514)
MSTNTTPSSWTNPLRDPQDKRLPRIAGPSGMVIFGVTGDLARKKLLPAIYDLANRGLL
PPGFSLVGYGRREWSKEDFEKYVRDAASAGARTEFRENVWERLAEGMEFVRGNFDD
DAAFDNLAATLKRIDKTRGTAGNWAYYLSIPPDSFTAVCHQLERSGMAESTEEAWRR
VIIEKPFGHNLESAHELNQLVNAVFPESSVFRIDHYLGKETVQNILALRFANQLFEPLW
NSNYVDHVQITMAEDIGLGGRAGYYDGIGAARDVIQNHLIQLLALVAMEEPISFVPAQ
LQAEKIKVLSATKPCYPLDKTSARGQYAAGWQGSELVKGLREEDGENPESTTETFAA
CTLEITSRRWAGVPFYLRTGKRLGRRVTEIAVVFKDAPHQPFDGDMTVSLGQNAIVIR
VQPDEGVLIRF
GSKVPGSAMEVRDVNMDFSYSESFTEESPEAYERLILDALLDESSLFPTNEEVELSWKI
LDPILEAWDADGEPEDYPAGTWGPKSADEMLSRNGHTWRRP*
SEQ ID No. 29; DNA; glucose-6-phosphate dehydrogenase of
Leuconoston mesenteroides, codon optimized mutant (R46E/Q47E)
of the gene having accession number M64446.1; lmZWF
ATGGTTTCCGAAATAAAGACCCTCGTTACTTTCTTTGGCGGCACCGGTGACCTTGC
AAAACGCAAGCTCTACCCCTCTGTATTCAACCTGTACAAAAAAGGGTATCTGCAA
AAACACTTCGCCATTGTTGGTACCGCTGAAGAGGCGCTAAACGACGACGAGTTCA
AACAGCTTGTCCGTGATTCCATTAAAGACTTCACCGATGACCAAGCCCAGGCAGA
GGCCTTCATCGAACATTTTTCTTATCGAGCACACGATGTGACCGATGCCGCATCGT
ATGCAGTCCTGAAGGAAGCGATCGAGGAGGCGGCCGATAAGTTCGATATTGACGG
TAACCGCATATTTTACATGTCGGTGGCACCACGCTTCTTCGGTACCATCGCTAAAT
ATCTGAAGTCCGAGGGTCTGCTTGCTGATACTGGCTACAATCGGCTGATGATTGAA
AAGCCTTTTGGAACCTCTTATGACACAGCCGCAGAACTACAGAATGACTTGGAGA
ACGCTTTCGATGATAATCAGCTTTTCCGTATCGATCATTATCTGGGTAAAGAAATG
GTCCAGAATATCGCAGCTCTGCGCTTCGGAAACCCTATATTCGACGCTGCGTGGAA
CAAGGATTACATCAAGAACGTTCAAGTTACACTCTCCGAAGTGTTGGGGGTTGAA
GAGCGAGCCGGCTACTATGACACCGCCGGAGCTCTACTTGATATGATCCAGAACC
ACACGATGCAGATCGTTGGCTGGCTCGCCATGGAAAAACCAGAGTCCTTCACCGA
TAAAGACATCCGCGCGGCTAAGAACGCCGCTTTTAATGCCCTGAAAATCTACGAC
GAGGCTGAAGTGAACAAATATTTTGTGCGTGCCCAATATGGTGCTGGAGATTCTGC
CGATTTCAAACCTTACTTAGAGGAACTCGATGTCCCAGCAGATTCCAAAAACAAC
ACCTTCATTGCAGGCGAATTACAGTTTGATCTTCCACGTTGGGAAGGTGTGCCTTT
TTACGTGCGCAGCGGTAAACGACTCGCAGCCAAACAGACTCGCGTCGATATTGTTT
TCAAGGCTGGCACCTTTAATTTCGGGTCTGAACAGGAAGCTCAAGAGGCCGTTCTG
TCCATCATCATTGATCCGAAGGGAGCAATCGAACTGAAGCTCAATGCAAAATCTG
TGGAAGATGCTTTCAACACCCGCACTATCGACTTGGGCTGGACCGTGAGCGATGA
GGACAAGAAAAATACGCCAGAACCTTATGAAAGGATGATCCACGATACGATGAA
CGGTGACGGCAGCAACTTCGCAGATTGGAATGGCGTGAGCATTGCGTGGAAGTTC
GTAGATGCTATTTCTGCGGTATATACGGCCGATAAGGCCCCGCTTGAAACCTACAA
GTCGGGCTCCATGGGACCCGAAGCCAGCGACAAATTGCTCGCCGCAAACGGAGAT
GCATGGGTATTCAAGGGGTAG
SEQ ID No. 30; PRT; mutant (R46E/Q47E) of the glucose-6-
phosphate dehydrogenase of Leuconoston.mesenteroides; lmZWF
MVSEIKTLVTFFGGTGDLAKRKLYPSVFNLYKKGYLQKHFAIVGTAEEALNDDEFKQ
LVRDSIKDFTDDQAQAEAFIEHFSYRAHDVTDAASYAVLKEAIEEAADKFDIDGNRIFY
MSVAPRFFGTIAKYLKSEGLLADTGYNRLMIEKPFGTSYDTAAELQNDLENAFDDNQL
FRIDHYLGKEMVQNIAALRFGNPIFDAAWNKDYIKNVQVTLSEVLGVEERAGYYDTA
GALLDMIQNHTMQIVGWLAMEKPESFTDKDIRAAKNAAFNALKIYDEAEVNKYFVR
AQYGAGDSADFKPYLEELDVPADSKNNTFIAGELQFDLPRWEGVPFYVRSGKRLAAK
QTRVDIVFKAGTFNFGSEQEAQEAVLSIIIDPKGAIELKLNAKSVEDAFNTRTIDLGWT
VSDEDKKNTPEPYERMIHDTMNGDGSNFADWNGVSIAWKFVDAISAVYTADKAPLE
TYKSGSMGPEASDKLLAANGDAWVFKG
SEQ ID No. 31; DNA; Codon optimized variant of the glucose-
6-phosphate dehydrogenase from Zymomonas mobilis strain
ATCC 10988 (Zmob_0908)
ATGACTAATACTGTTTCTACCATGATCCTTTTCGGCAGCACCGGAGATCTCTCGCA
GCGCATGCTTCTTCCCTCGCTGTACGGGCTGGATGCAGACGGTCTACTCGCCGACG
ACCTCCGCATTGTGTGTACCTCTCGTTCCGAGTACGATACCGACGGATTTCGTGAT
TTTGCTGAGAAGGCACTGGACCGTTTCGTTGCCTCCGACAGACTTAATGATGATGC
AAAAGCGAAGTTCCTCAACAAGCTTTTCTACGCAACGGTTGACATCACCGATCCA
ACCCAATTTGGAAAGCTCGCAGACCTCTGCGGTCCAGTCGAAAAGGGCATTGCAA
TCTACCTTTCCACAGCACCATCCTTGTTCGAAGGCGCAATTGCTGGCTTGAAACAG
GCGGGCCTGGCCGGCCCGACCTCCCGCCTTGCATTGGAAAAGCCCTTGGGTCAAG
ATCTTGCTTCCTCTGATCACATCAACGACGCAGTGCTGAAGGTTTTTTCCGAAAAA
CAAGTATACCGTATCGACCACTATCTTGGGAAAGAAACCGTCCAGAATCTCCTAA
CACTCCGCTTTGGAAATGCATTGTTCGAGCCGTTGTGGAACTCAAAGGGGATTGAC
CACGTGCAGATCTCCGTCGCTGAGACAGTGGGACTCGAAGGACGCATCGGCTACT
TTGACGGCTCCGGCTCCCTGCGAGACATGGTGCAGTCTCACATCCTGCAATTGGTT
GCCCTTGTAGCTATGGAGCCCCCGGCTCACATGGAAGCAAACGCGGTCCGCGACG
AAAAGGTTAAGGTGTTCCGTGCACTTCGTCCCATTAACAACGACACTGTTTTCACA
CACACCGTGACTGGCCAATACGGCGCCGGCGTGTCGGGGGGAAAGGAAGTTGCAG
GCTACATCGATGAGCTTGGACAACCGAGTGATACTGAAACCTTTGTTGCAATTAAA
GCACACGTGGATAACTGGCGCTGGCAGGGAGTTCCCTTCTACATCCGCACTGGTA
AACGGCTCCCTGCCCGCCGTTCAGAGATCGTCGTTCAGTTCAAACCAGTTCCCCAC
TCCATTTTTTCAAGCTCAGGAGGAATCCTTCAGCCTAATAAATTGCGCATTGTCCT
GCAACCAGACGAAACCATCCAAATCTCAATGATGGTCAAGGAACCAGGTCTTGAC
AGAAATGGTGCACACATGCGTGAGGTCTGGCTGGATCTCTCTTTGACCGACGTGTT
CAAAGATCGAAAGCGCCGGATTGCTTACGAGCGCCTTATGCTCGATCTGATTGAG
GGTGACGCAACCCTCTTCGTGCGCCGCGACGAGGTCGAGGCACAGTGGGTTTGGA
TCGACGGTATCCGGGAAGGCTGGAAGGCTAATAGCATGAAGCCTAAAACCTATGT
CTCCGGCACCTGGGGACCCTCCACCGCTATTGCATTGGCAGAGCGCGATGGCGTC
ACCTGGTACGACTAA
SEQ ID No. 32; PRT; codon-optimized variant of the glu6-
phosphate dehydrogenase from Zymomonas mobilis strain
ATCC 10988 (NCBI-ProteinID: AEH62743.1)
MTNTVSTMILFGSTGDLSQRMLLPSLYGLDADGLLADDLRIVCTSRSEYDTDGFRDFA
EKALDRFVASDRLNDDAKAKFLNKLFYATVDITDPTQFGKLADLCGPVEKGIAIYLST
APSLFEGAIAGLKQAGLAGPTSRLALEKPLGQDLASSDHINDAVLKVFSEKQVYRIDH
YLGKETVQNLLTLRFGNALFEPLWNSKGIDHVQISVAETVGLEGRIGYFDGSGSLRDM
VQSHILQLVALVAMEPPAHMEANAVRDEKVKVFRALRPINNDTVFTHTVTGQYGAG
VSGGKEVAGYIDELGQPSDTETFVAIKAHVDNWRWQGVPFYIRTGKRLPARRSEIVVQ
FKPVPHSIFSSSGGILQPNKLRIVLQPDETIQISMMVKEPGLDRNGAHMREVWLDLSLT
DVFKDRKRRIAYERLMLDLIEGDATLFVRRDEVEAQWVWIDGIREGWKANSMKPKT
YVSGTWGPSTAIALAERDGVTWYD*
SEQ ID No. 33; DNA; soluble Pyridine Nucleotide Transhydrogenase
from Escherichia coli; udhA (NP_418397.2, EG11428)
ATGCCACATTCCTACGATTACGATGCCATAGTAATAGGTTCCGGCCCCGGCGGCGA
AGGCGCTGCAATGGGCCTGGTTAAGCAAGGTGCGCGCGTCGCAGTTATCGAGCGT
TATCAAAATGTTGGCGGCGGTTGCACCCACTGGGGCACCATCCCGTCGAAAGCTCT
CCGTCACGCCGTCAGCCGCATTATAGAATTCAATCAAAACCCACTTTACAGCGACC
ATTCCCGACTGCTCCGCTCTTCTTTTGCCGATATCCTTAACCATGCCGATAACGTGA
TTAATCAACAAACGCGCATGCGTCAGGGATTTTACGAACGTAATCACTGTGAAAT
ATTGCAGGGAAACGCTCGCTTTGTTGACGAGCATACGTTGGCGCTGGATTGCCTGG
ACGGCAGCGTTGAAACACTAACCGCTGAAAAATTTGTTATTGCCTGCGGCTCTCGT
CCATATCATCCAACAGATGTTGATTTCACCCATCCACGCATTTACGACAGCGACTC
AATTCTCAGCATGCACCACGAACCGCGCCATGTACTTATCTATGGTGCTGGAGTGA
TCGGCTGTGAATATGCGTCGATCTTCCGCGGTATGGATGTAAAAGTGGATCTGATC
AACACCCGCGATCGCCTGCTGGCATTTCTCGATCAAGAGATGTCAGATTCTCTCTC
CTATCACTTCTGGAACAGTGGCGTAGTGATTCGTCACAACGAAGAGTACGAGAAG
ATCGAAGGCTGTGACGATGGTGTGATCATGCATCTGAAGTCGGGTAAAAAACTGA
AAGCTGACTGCCTGCTCTATGCCAACGGTCGCACCGGTAATACCGATTCGCTGGCG
TTACAGAACATTGGGCTAGAAACTGACAGCCGCGGACAGCTGAAGGTCAACAGCA
TGTATCAGACCGCACAGCCACACGTTTACGCGGTGGGCGACGTGATTGGTTATCCG
AGCCTGGCGTCGGCGGCCTATGACCAGGGGCGCATTGCCGCGCAGGCGCTGGTAA
AAGGCGAAGCCACCGCACATCTGATTGAAGATATCCCTACCGGTATTTACACCATC
CCGGAAATCAGCTCTGTGGGCAAAACCGAACAGCAGCTGACCGCAATGAAAGTGC
CATATGAAGTGGGCCGCGCCCAGTTTAAACATCTGGCACGCGCACAAATCGTCGG
CATGAACGTGGGCACGCTGAAAATTTTGTTCCATCGGGAAACAAAAGAGATTCTG
GGTATTCACTGCTTTGGCGAGCGCGCTGCCGAAATTATTCATATCGGTCAGGCGAT
TATGGAACAGAAAGGTGGCGGCAACACTATTGAGTACTTCGTCAACACCACCTTT
AACTACCCGACGATGGCGGAAGCCTATCGGGTAGCTGCGTTAAACGGTTTAAACC
GCCTGTTTTAA
SEQ ID No. 34; PRT; soluble Pyridine Nucleotide Transhydrogenase
from Escherichia coli; udhA (AAC76944)
MPHSYDYDAIVIGSGPGGEGAAMGLVKQGARVAVIERYQNVGGGCTHWGTIPSKAL
RHAVSRIIEFNQNPLYSDHSRLLRSSFADILNHADNVINQQTRMRQGFYERNHCEILQG
NARFVDEHTLALDCLDGSVETLTAEKFVIACGSRPYHPTDVDFTHPRIYDSDSILSMHH
EPRHVLIYGAGVIGCEYASIFRGMDVKVDLINTRDRLLAFLDQEMSDSLSYHFWNSGV
VIRHNEEYEKIEGCDDGVIMHLKSGKKLKADCLLYANGRTGNTDSLALQNIGLETDSR
GQLKVNSMYQTAQPHVYAVGDVIGYPSLASAAYDQGRIAAQALVKGEATAHLIEDIP
TGIYTIPEISSVGKTEQQLTAMKVPYEVGRAQFKHLARAQIVGMNVGTLKILFHRETKE
ILGIHCFGERAAEIIHIGQAIMEQKGGGNTIEYFVNTTFNYPTMAEAYRVAALNGLNRL
F
SEQ ID No. 35; DNA; “A” subunit of Membrane bound Pyridine
Nucleotide Transhydrogenase (pnt) from Escherichia coli
MG1655; ECK1598 (variant g1342a, NP_416120.1)
ATGCCACATTCCTACGATTACGATGCCATAGTAATAGGTTCCGGCCCCGGCGGCGA
AGGCGCTGCAATGGGCCTGGTTAAGCAAGGTGCGCGCGTCGCAGTTATCGAGCGT
TATCAAAATGTTGGCGGCGGTTGCACCCACTGGGGCACCATCCCGTCGAAAGCTCT
CCGTCACGCCGTCAGCCGCATTATAGAATTCAATCAAAACCCACTTTACAGCGACC
ATTCCCGACTGCTCCGCTCTTCTTTTGCCGATATCCTTAACCATGCCGATAACGTGA
TTAATCAACAAACGCGCATGCGTCAGGGATTTTACGAACGTAATCACTGTGAAAT
ATTGCAGGGAAACGCTCGCTTTGTTGACGAGCATACGTTGGCGCTGGATTGCCTGG
ACGGCAGCGTTGAAACACTAACCGCTGAAAAATTTGTTATTGCCTGCGGCTCTCGT
CCATATCATCCAACAGATGTTGATTTCACCCATCCACGCATTTACGACAGCGACTC
AATTCTCAGCATGCACCACGAACCGCGCCATGTACTTATCTATGGTGCTGGAGTGA
TCGGCTGTGAATATGCGTCGATCTTCCGCGGTATGGATGTAAAAGTGGATCTGATC
AACACCCGCGATCGCCTGCTGGCATTTCTCGATCAAGAGATGTCAGATTCTCTCTC
CTATCACTTCTGGAACAGTGGCGTAGTGATTCGTCACAACGAAGAGTACGAGAAG
ATCGAAGGCTGTGACGATGGTGTGATCATGCATCTGAAGTCGGGTAAAAAACTGA
AAGCTGACTGCCTGCTCTATGCCAACGGTCGCACCGGTAATACCGATTCGCTGGCG
TTACAGAACATTGGGCTAGAAACTGACAGCCGCGGACAGCTGAAGGTCAACAGCA
TGTATCAGACCGCACAGCCACACGTTTACGCGGTGGGCGACGTGATTGGTTATCCG
AGCCTGGCGTCGGCGGCCTATGACCAGGGGCGCATTGCCGCGCAGGCGCTGGTAA
AAGGCGAAGCCACCGCACATCTGATTGAAGATATCCCTACCGGTATTTACACCATC
CCGGAAATCAGCTCTGTGGGCAAAACCGAACAGCAGCTGACCGCAATGAAAGTGC
CATATGAAGTGGGCCGCGCCCAGTTTAAACATCTGGCACGCGCACAAATCGTCGG
CATGAACGTGGGCACGCTGAAAATTTTGTTCCATCGGGAAACAAAAGAGATTCTG
GGTATTCACTGCTTTGGCGAGCGCGCTGCCGAAATTATTCATATCGGTCAGGCGAT
TATGGAACAGAAAGGTGGCGGCAACACTATTGAGTACTTCGTCAACACCACCTTT
AACTACCCGACGATGGCGGAAGCCTATCGGGTAGCTGCGTTAAACGGTTTAAACC
GCCTGTTTTAA
SEQ ID No. 36; PRT; “A” subunit of Membrane bound Pyridine
Nucleotide Transhydrogenase (pnt) from Escherichia coli;
P07001.2 (A434T variant)
MRIGIPRERLTNETRVAATPKTVEQLLKLGFTVAVESGAGQLASFDDKAFVQAGAEIV
EGNSVWQSEIILKVNAPLDDEIALLNPGTTLVSFIWPAQNPELMQKLAERNVTVMAM
DSVPRISRAQSLDALSSMANIAGYRAIVEAAHEFGRFFTGQITAAGKVPPAKVMVIGA
GVAGLAAIGAANSLGAIVRAFDTRPEVKEQVQSMGAEFLELDFKEEAGSGDGYAKV
MSDAFIKAEMELFAAQAKEVDIIVTTALIPGKPAPKLITREMVDSMKAGSVIVDLAAQ
NGGNCEYTVPGEIFTTENGVKVIGYTDLPGRLPTQSSQLYGTNLVNLLKLLCKEKDGN
ITVDFDDVVIRGVTVIRAGEITWPAPPIQVSAQPQAAQKAAPEVKTEEKCTCSPWRKY
ALMALAIILFGWMASVAPKEFLGHFTVFALTCVVGYYVVWNVSHALHTPLMSVTNAI
SGIIVVGALLQIGQGGWVSFLSFIAVLIASINIFGGFTVTQRMLKMFRKN
SEQ ID No. 37; DNA; “B” subunit of Membrane bound Pyridine
Nucleotide Transhydrogenase (pntB) from Escherichia coli
(MG1655; ECK1597; NP_416119.1)
ATGTCTGGAGGATTAGTTACAGCTGCATACATTGTTGCCGCGATCCTGTTTATCTTC
AGTCTGGCCGGTCTTTCGAAACATGAAACGTCTCGCCAGGGTAACAACTTCGGTAT
CGCCGGGATGGCGATTGCGTTAATCGCAACCATTTTTGGACCGGATACGGGTAAT
GTTGGCTGGATCTTGCTGGCGATGGTCATTGGTGGGGCAATTGGTATCCGTCTGGC
GAAGAAAGTTGAAATGACCGAAATGCCAGAACTGGTGGCGATCCTGCATAGCTTC
GTGGGTCTGGCGGCAGTGCTGGTTGGCTTTAACAGCTATCTGCATCATGACGCGGG
AATGGCACCGATTCTGGTCAATATTCACCTGACGGAAGTGTTCCTCGGTATCTTCA
TCGGGGCGGTAACGTTCACGGGTTCGGTGGTGGCGTTCGGCAAACTGTGTGGCAA
GATTTCGTCTAAACCATTGATGCTGCCAAACCGTCACAAAATGAACCTGGCGGCTC
TGGTCGTTTCCTTCCTGCTGCTGATTGTATTTGTTCGCACGGACAGCGTCGGCCTGC
AAGTGCTGGCATTGCTGATAATGACCGCAATTGCGCTGGTATTCGGCTGGCATTTA
GTCGCCTCCATCGGTGGTGCAGATATGCCAGTGGTGGTGTCGATGCTGAACTCGTA
CTCCGGCTGGGCGGCTGCGGCTGCGGGCTTTATGCTCAGCAACGACCTGCTGATTG
TGACCGGTGCGCTGGTCGGTTCTTCGGGGGCTATCCTTTCTTACATTATGTGTAAG
GCGATGAACCGTTCCTTTATCAGCGTTATTGCGGGTGGTTTCGGCACCGACGGCTC
TTCTACTGGCGATGATCAGGAAGTGGGTGAGCACCGCGAAATCACCGCAGAAGAG
ACAGCGGAACTGCTGAAAAACTCCCATTCAGTGATCATTACTCCGGGGTACGGCA
TGGCAGTCGCGCAGGCGCAATATCCTGTCGCTGAAATTACTGAGAAATTGCGCGC
TCGTGGTATTAATGTGCGTTTCGGTATCCACCCGGTCGCGGGGCGTTTGCCTGGAC
ATATGAACGTATTGCTGGCTGAAGCAAAAGTACCGTATGACATCGTGCTGGAAAT
GGACGAGATCAATGATGACTTTGCTGATACCGATACCGTACTGGTGATTGGTGCTA
ACGATACGGTTAACCCGGCGGCGCAGGATGATCCGAAGAGTCCGATTGCTGGTAT
GCCTGTGCTGGAAGTGTGGAAAGCGCAGAACGTGATTGTCTTTAAACGTTCGATG
AACACTGGCTATGCTGGTGTGCAAAACCCGCTGTTCTTCAAGGAAAACACCCACA
TGCTGTTTGGTGACGCCAAAGCCAGCGTGGATGCAATCCTGAAAGCTCTGTAA
SEQ ID No. 38; “B” subunit of Membrane bound Pyridine
Nucleotide Transhydrogenase (pntB) from Escherichia coli;
P0AB69.1
MSGGLVTAAYIVAAILFIFSLAGLSKHETSRQGNNFGIAGMAIALIATIFGPDTGNVGWI
LLAMVIGGAIGIRLAKKVEMTEMPELVAILHSFVGLAAVLVGFNSYLHHDAGMAPILV
NIHLTEVFLGIFIGAVTFTGSVVAFGKLCGKISSKPLMLPNRHKMNLAALVVSFLLLIVF
VRTDSVGLQVLALLIMTAIALVFGWHLVASIGGADMPVVVSMLNSYSGWAAAAAGF
MLSNDLLIVTGALVGSSGAILSYIMCKAMNRSFISVIAGGFGTDGSSTGDDQEVGEHRE
ITAEETAELLKNSHSVIITPGYGMAVAQAQYPVAEITEKLRARGINVRFGIHPVAGRLP
GHMNVLLAEAKVPYDIVLEMDEINDDFADTDTVLVIGANDTVNPAAQDDPKSPIAGM
PVLEVWKAQNVIVFKRSMNTGYAGVQNPLFFKENTHMLFGDAKASVDAILKAL

1. A genetically modified microbial cell capable of producing nicotinamide mononucleotide and/or nicotinamide adenine dinucleotide from nicotinamide, said cell comprising a mutation in one or more endogenous genes selected from the group consisting of cgl1364, cgl1977, cgl2835, iunH1, iunH2, and iunH3, wherein each of said one or more genes codes for a nucleosidase enzyme.

2. The genetically modified microbial cell as in claim 1, wherein the mutation is a deletion, frameshift or point mutation decreasing or eliminating nucleosidase activity.

3. The genetically modified microbial cell as in claim 1, wherein the microbial cell is Corynebacteriun glutamicum.

4. The genetically modified microbial cell as in claim 1, wherein the microbial cell is Corynebacterium glutamicum ATCC 13034.

5. The genetically modified microbial cell as in claim 1, further comprising a mutation in the pncA gene, wherein such mutation leads to the inactivation or reduced activity of an enzyme capable of deamidation of nicotinamide to nicotinic acid.

6. The genetically modified microbial cell as in claim 5, wherein the mutation is the deletion, frameshift or point mutation of the pncA gene decreasing or eliminating deamidation activity.

7. The genetically modified microbial cell as in claim 1, further comprising an exogenous gene nadV coding for a nicotinamide phosphoribosyl transferase capable of the conversion of nicotinamide to nicotinamide mononucleotide.

8. The genetically modified microbial cell as in claim 7, wherein:
(i) the exogenous gene nadV is from Stenophomonas maltophilia;
(ii) the exogenous gene nadV is from Chromobacterium violaceum;
(iii) the genetically modified microbial cell further comprises a prsA gene variant or mutant encoding a phosphoribosyl pyrophosphate synthelase;
(iv) the genetically modified microbial cell further comprises a genetic modification to an endogenous pyrE gene, leading to the reduced or eliminated expression of a phosphoribosyltransferase enzyme;
(v) a mutation in an endogenous ushA gene causing inactivation or reduction in activity of a purine nucleosidase enzyme capable of the conversion of nicotinamide mononucleotide to nicotinamide riboside;
(vi) a genetic modification in an endogenous pncC gene coding for a nicotinamide nucleotide amidase enzyme capable of the conversion of nicotinamide mononucleotide to nicotinate mononucleotide, causing inactivation or reduction in activity;
(vii) a genetic modification in an endogenous nadD gene, leading to the upregulation of a nicotinate-nucleotide adenyl transferase enzyme capable of the conversion of nicotinamide mononucleotide to nicotinamide adenine dinucleotide; or
(viii) the expression of a heterologous gene encoding a nicotinate-nucleotide adenyl transferase enzyme capable of the conversion of nicotinamide mononucleotide to nicotinamide adenine dinucleotide.

9. The genetically modified microbial cell as in claim 8, wherein the gene nadV from Stenophomonas maltophilia is codon optimized for expression in Corynebacterium glutamicum.

10. (canceled)

11. The genetically modified microbial cell as in claim 8, wherein the gene nadV from Chromobacterium violaceum is codon optimized for expression in Corynebacterium glutamicum.

12. (canceled)

13. The genetically modified microbial cell as in claim 8, wherein:
(i) the prsA gene variant or mutant is a codon optimized variant of the prsA gene from Corynebacterium glutamicum ATCC 13032; or
(ii) the prsA gene variant or mutant encodes a feedback resistant mutant of phosphoribosyl pyrophosphate synthetase.

14.-19. (canceled)

20. The genetically modified microbial cell as in claim 8, further comprising a genetic modification in an endogenous nudC gene, leading to the reduced or eliminated expression of an NADH pyrophosphatase capable of the conversion of NAD(+) to nicotinamide mononucleotide.

21. The genetically modified microbial cell as in claim 9, further comprising a conditionally active ribonucleotide reductase coded by a NrdHIEJ operon.

22. The genetically modified microbial cell as in claim 1, further comprising a mutation in the pgi gene, wherein such mutation leads to the inactivation or reduced activity of an enzyme capable of converting D-glucose-6-phosphate to D-ribulose-5-phosphate.

23. The genetically modified microbial as in claim 22, wherein:
(i) the mutation is the deletion, frameshift or point mutation of the pgi gene decreasing or eliminating conversion of D-glucose-6-phosphate to D-ribulose-5-phosphate;
(ii) the genetically modified microbial cell further comprises an exogenous gene coding for a soluble transhydrogenase; or
(iii) the genetically modified microbial cell further comprises an exogenous gene coding for a membrane-bound transhydrogenase.

24. (canceled)

25. The genetically modified microbial cell as in claim 23, wherein;
(i) the exogenous gene is a udhA gene from E. coli; or
(ii) the exogenous gene is a pntAB gene from E. coli.

26. The genetically modified microbial cell as in claim 25, wherein the gene udhA from E. coli is codon optimized for expression in Corynebacterium glutamicum.

27.-28. (canceled)

29. The genetically modified microbial cell as in claim 25, wherein the gene pntAB from E. coli is codon optimized for expression in Corynebacterium glutamicum.

30. A method for producing nicotinamide mononucleotide comprising: culturing a genetically modified microbial cell as in claim 1 in the presence nicotinamide for a sufficient period of time to allow conversion of nicotinamide to nicotinamide mononucleotide.

31. A method for producing nicotinamide adenine dinucleotide comprising: culturing a genetically modified microbial cell as in claim 8 in the presence of nicotinamide for a sufficient period of time to allow conversion of nicotinamide to nicotinamide adenine dinucleotide.


SEQUENCE LISTING (38 sequences)

SEQ ID NO: 1 - protein, 561 aa, Chromobacterium violaceum
MTAPKAAQDKNQLVPFNLADFYKTGHPAMYPRETTRLVANFTPRSAKYAQVLPQLFDDKVVWFGLQGFIQEYLIDLFNREFFQRPKADAVRRYQRRMDTALGAGAVDGGRLEALHDLGHLPLEIRSLPEGARVDIKVPPVTFSNTHPDFPWVATYFETLFSCESWKPSTVATIAFEFRKLLSYFAALTGAPQDFVAWQGHDFSMRGMSGVHDAMRCGAGHLLSFTGTDTIPALDYLEDHYGADAERELVGGSIPASEHSVMALRILLTQQRLARMPAHQGLDDKALRRLAEREVVREFVTRDYPAGMVSIVSDTFDFWNVLTVIARELKDDIQARRPDALGNAKVVFRPDSGDPVRILAGYRDDELQFDDAGNCTARDDGRPVSAAERKGAVECLWDIFGGTVTERGYRVLDSHVGLIYGDSITLPRARDILLRLAEKGYASCNVVFGIGSFVYGMNSRDTFGYALKAVYAEVAGEAVDIYKDPATDDGTKKSARGLLRVEEENGRYALYQQQTPAEAEGGALRPVFRDGELLVKQTLAEIRQRLQASWTCPEAGSIVWNA

SEQ ID NO: 2 - protein, 469 aa, Stenotrophomonas maltophilia
MHYLDNLLLNTDSYKASHWLQYPPGTDATFFYVESRGGLHDRTVFFGLQAILKDALARPVTHADIDDAAAVFAAHGEPFNEAGWRDIVDRLGGHLPVRIRAVPEGSVVPTHQALMTIESTDPAAFWVPSYLETLLLRVWYPVTVATISWHARQTIAAFLQQTSDDPQGQLPFKLHDFGARGVSSLESAALGGAAHLVNFLGTDTVSALCLARAHYHAPMAGYSIPAAEHSTITSWGREREVDAYRNMLRQFGKPGSIVAVVSDSYDIYRAISEHWGTTLRDDVIASGATLVIRPDSGDPVEVVAESLRRLDEAFGHAINGKGYRVLNHVRVIQGDGINPDTIRAILQRITDDGYAADNVAFGMGGALLQRLDRDTQKFALKCSAARVEGEWIDVYKDPVTDAGKASKRGRMRLLRRLDDGSLHTVPLPANGDDTLPDGFEDAMVTVWENGHLLYDQRLDDIRTRAAVGH

SEQ ID NO: 3 - protein, 325 aa, unidentified
  REGION 1..325: note = Codon-optimized PRPP synthase variant, CYL77_RS04805 - prsA (PRS)
MTAHWKQNQKNLMLFSGRAHPELAEAVAKELDVNVTPMTARDFANGEIYVRFEESVRGSDCFVLQSHTQPLNKWLMEQLLMIDALKRGSAKRITAILPFYPYARQDKKHRGREPISARLIADLMLTAGADRIVSVDLHTDQIQGFFDGPVDHMHAMPILTDHIKENYNLDNICVVSPDAGRVKVAEKWANTLGDAPMAFVHKTRSTEVANQVVANRVVGDVDGKDCVLLDDMIDTGGTIAGAVGVLKKAGAKSVVIACTHGVFSDPARERLSACGAEEVITTDTLPQSTEGWSNLTVLSIAPLLARTINEIFENGSVTTLFEGEA

SEQ ID NO: 4 - protein, 325 aa, unidentified
  REGION 1..325: note = Feedback resistant PRPP synthase mutant, prsA L136I (PRS L136I)
MTAHWKQNQKNLMLFSGRAHPELAEAVAKELDVNVTPMTARDFANGEIYVRFEESVRGSDCFVLQSHTQPLNKWLMEQLLMIDALKRGSAKRITAILPFYPYARQDKKHRGREPISARLIADLMLTAGADRIVSVDIHTDQIQGFFDGPVDHMHAMPILTDHIKENYNLDNICVVSPDAGRVKVAEKWANTLGDAPMAFVHKTRSTEVANQVVANRVVGDVDGKDCVLLDDMIDTGGTIAGAVGVLKKAGAKSVVIACTHGVFSDPARERLSACGAEEVITTDTLPQSTEGWSNLTVLSIAPLLARTINEIFENGSVTTLFEGEA

SEQ ID NO: 5 - DNA, 561 bp, unidentified
  misc_feature 1..561: note = Nicotinamidase pncA (CGL_RS12350, ncgl2401, cgl2487)
  source 1..561: mol_type = other DNA
atggcacgcgcactcattctggttgatgttcaaaaagacttctgccccggtggcagcctagccaccgaacgaggcgatgaagtggcgggaaaaatcggtgcctatcagctgtcccacggctcagagtacgacgtcgttgtggcgacccaagattggcacatcgatccaggcgagcacttttcagaaaccccagactttaaaaactcctggccaatccactgcgtcgcggattccgatggtgccgccatgcatgaccgcatcaacaccgattcaatcgatgagttcttccgcaaaggccattacaccgcggcgtattccgggttcgagggaactgcagtcagtgaagaactcctcatgtctccatggctgaagaacaagggagtcactgatgtagacatcgtagggatcgctacggatcactgcgttcgagccacagcacttgatgctctcaaggagggcttcaacgtctccattttgacgtcgatgtgttctgcggtggatttccatgcgggagaccacgctttggaggaactacatgaagccggggcgattctgatttaa

SEQ ID NO: 6 - protein, 186 aa, unidentified
  REGION 1..186: note = Nicotinamidase pncA (CGL_RS12350, ncgl2401, cgl2487)
MARALILVDVQKDFCPGGSLATERGDEVAGKIGAYQLSHGSEYDVVVATQDWHIDPGEHFSETPDFKNSWPIHCVADSDGAAMHDRINTDSIDEFFRKGHYTAAYSGFEGTAVSEELLMSPWLKNKGVTDVDIVGIATDHCVRATALDALKEGFNVSILTSMCSAVDFHAGDHALEELHEAGAILI

SEQ ID NO: 7 - DNA, 519 bp, unidentified
  misc_feature 1..519: note = Nicotinamide-nucleotide amidohydrolase pncC (CGL_RS09770, ncgl1888, cgl1963)
  source 1..519: mol_type = other DNA
atgtcggagaatctggcggggcgagtggtggagctgttgaaatcgcgcggtgaaacgctggcgttttgtgaatccctcaccgccggccttgccagtgcgacgatcgcagagatccccggcgcctcagtggtacttaaaggcgggctggtcacctatgccaccgagcttaaggttgcgcttgccggtgtgccgcaggagcttatcgacgcgcacggcgttgtttccccgcagtgcgcccgtgcgatggcaacgggggccgcacacagatgccaggcagattgggcggtttcgctcacgggcgttgctggccccagcaaacaagatggtcatccggtgggggaagtgtggatcggagtggctggtcctgcgcattttggggcgtcgggaacaattgacgcgtatcgtgcgtttgaaagtgaacaacaggtaatattggctgaattgggacggcatcatattagagagtctgctgtgcagcaaagctttcgcctgctgattgaccatattgagtcgcagtga

SEQ ID NO: 8 - protein, 172 aa, unidentified
  REGION 1..172: note = Nicotinamide-nucleotide amidohydrolase pncC (CGL_RS09770, ncgl1888, cgl1963)
MSENLAGRVVELLKSRGETLAFCESLTAGLASATIAEIPGASVVLKGGLVTYATELKVALAGVPQELIDAHGVVSPQCARAMATGAAHRCQADWAVSLTGVAGPSKQDGHPVGEVWIGVAGPAHFGASGTIDAYRAFESEQQVILAELGRHHIRESAVQQSFRLLIDHIESQ

SEQ ID NO: 9 - DNA, 1077 bp, unidentified
  misc_feature 1..1077: note = Restriction modification system; RM= cgl1777 (CYL77_RS08985)
  source 1..1077: mol_type = other DNA
atgaagcccaccgttaatgttgtgttcaatgcgcatcaccccaaagatacgcagccgttggataagttcttcgataaagaacttaaagacacacatcatctcgatataacggtgggttatatcagtgagaaatcactacaatatttgcttcttattgcaggcactcaccccgacctcaccatcacactcacctgtggaatgcacgctcgtgaaggcatgactgctgcccaactgcatcatgcgcgagtgctccatgactacttaagcgaccatgatcgaggcggggtgttcgttattccccgattgcgttatcacggcaaaatctatcttttccacaagaaccagcacacagatcctattgcttatatcggtagcgctaacctctcagccatcgttcctgggtacacctctacattcgagaccggcgtcatcttagaccccgcacctgaagatctcgtgcttcatctcaaccgtgatgtcgtacccctatgtgtccccattgacaccgcgcatgtccccatcattaaagatcaagaatccccgatgaagcacgtcgctgaagcaacagctgtgtccacctctgatgttgttgccatcatgtccagcccatttacttatagttttgaccttaaactcaaagccactgccagcagcaacctcaatgctcataactcaggcggtggcgcgcgcaaacagaaaaacggtagcttccttgcacgcaattggtatgagggcgaaatcattgtcggtgtcgagacaacaagactcccaggttacccacaaaacaaatccgaattcactgcggtcactgatgacggctggtcatttgtttgcaaaatcagcggaggaaacggaaagaacctacgcagcaaaggtgacctgtccatcctcggtacgtggttaaagtctcgattcattgaacaaggtgccctggaatacggcgaggatgccacccaagaaaacatcgaccgttttgggagaacacatatgaccatgcgctatcacccagatttcgatgtgtggtcattcgatctcagccaaaccccgaagccttcgacacagattgggcaggattaa

SEQ ID NO: 10 - protein, 358 aa, unidentified
  REGION 1..358: note = Restriction modification system; RM= cgl1777 (CYL77_RS08985)
MKPTVNVVFNAHHPKDTQPLDKFFDKELKDTHHLDITVGYISEKSLQYLLLIAGTHPDLTITLTCGMHAREGMTAAQLHHARVLHDYLSDHDRGGVFVIPRLRYHGKIYLFHKNQHTDPIAYIGSANLSAIVPGYTSTFETGVILDPAPEDLVLHLNRDVVPLCVPIDTAHVPIIKDQESPMKHVAEATAVSTSDVVAIMSSPFTYSFDLKLKATASSNLNAHNSGGGARKQKNGSFLARNWYEGEIIVGVETTRLPGYPQNKSEFTAVTDDGWSFVCKISGGNGKNLRSKGDLSILGTWLKSRFIEQGALEYGEDATQENIDRFGRTHMTMRYHPDFDVWSFDLSQTPKPSTQIGQD

SEQ ID NO: 11 - DNA, 1886 bp, unidentified
  misc_feature 1..1886: note = Restriction modification system; RM= cgl1778 (CYL77_RS08990)
  source 1..1886: mol_type = other DNA
rtcacctcaataactacatcacgagcttgagtgataacgctgatctccgtgaaaaagtcaccgcaaccgtagacgctttccgccataccgtcatggatgacttcgactacatcagtgatcaacaagtcctgctttatggcgatgtccaaagcggtaaaacctcacacatgctgggaattatcgcagattgcctcgacagtacgtttcacaccattgttattctgacctcgcctaacacacggctcgtgcaacaaacatacgaccgtgttgcccaagcatttccagatactttggtgtgcgaccgtgacggatacaatgatttccgtgcgaatcaaaagagcctcaccccgcgaaaatctatcgtagtcgtcggaaaaatacctgcagttcttggtaattggttacgcgtctttaacgacagtggcgcactttctggacaccctgtactcattattgatgacgaagcagatgcgacaagtctcaacaccaaagtaaatcagtctgatgtttcgaccattaaccaccagctcactagcataagagaccttgccacaggatgcatctaccttcaggtcacaggtacacctcaagcggtgcttcttcaaagcgacgatagcaactgggcagcggaacatgtgcttcacttcgcacctggtgagagctacatcggtggtcaacttttcttttctgagctcaacaacccttatctacgacttttcgctaatacccaatttgacgaggattctcgcttcagcgacgccatttacacctatctcttaaccgcagcactgttcaaacttcgcggtgaaagcttgtgtaccatgctcattcaccccagccacactgcatccagtcatagagacttcgcgcaagaagcccgcctccaactcactttcgccttcgagcgattctatgaaccaatgattcagcacaatttccaacgtgcttatgaacagctcgcacaaactgacagcaacctgccacccttgagaaaaattcttaacattcttggtggcatggaagatgacttctccatccacatcgtcaatagcgacaacccgactgttgaggaagattgggctgatggttataacattattgtcggtggcaactcgcttgggcgcggtttaacattcaacaacttgcaaaccgttttctacgtgcgcgaatccaagcgaccacaagcagacaccctgtggcagcacgcccgcatgtttggctacaaacgccacaaagacaccatgcgtgtgttcatgccggccactattgctcaaaccttccaagaggtctatctcggcaacgaagctattaaaaatcagctcgatcatggcacgcatatcaacgacattcgggtcattttaggtgatggcgtcgcacctactcgtgccaatgttctcgacaaacgcaaagttggaaacctcagcggtggcgtcaactactttgccgctgatcctagaatcaagaatgtcgaagcactcgacaaaaaactcttggcctacttagacaagcacggtgaggactccaccatcggtatgcgcgcgataatcaccattctcaacgcctttactgtagaccccaacgatctcgacctcgcgaccttcaaggctgcgctccttgactttgaacgcaaccaacctcatctcacagcacgtatggtgctgcgaacaaaccgcaaagtcaatcagggtacaggcgccctgctctcccctactgatcaagctctcagccgtgcagaagtcgcacacccattattgatcctataccgcattgaaggtgttaacgatgctgctgcgcaacgaggtgaacctacgtggtcaagcgaccctatctgggtgcctaatattaaactccctggtcaacgtcaattctggtgcgtagacggctaa

SEQ ID NO: 12 - protein, 632 aa, unidentified
  REGION 1..632: note = Restriction modification system; RM= cgl1778 (CYL77_RS08990)
MSHHTHLNNYITSLSDNADLREKVTATVDAFRHTVMDDFDYISDQQVLLYGDVQSGKTSHMLGIIADCLDSTFHTIVILTSPNTRLVQQTYDRVAQAFPDTLVCDRDGYNDFRANQKSLTPRKSIVVVGKIPAVLGNWLRVFNDSGALSGHPVLIIDDEADATSLNTKVNQSDVSTINHQLTSIRDLATGCIYLQVTGTPQAVLLQSDDSNWAAEHVLHFAPGESYIGGQLFFSELNNPYLRLFANTQFDEDSRFSDAIYTYLLTAALFKLRGESLCTMLIHPSHTASSHRDFAQEARLQLTFAFERFYEPMIQHNFQRAYEQLAQTDSNLPPLRKILNILGGMEDDFSIHIVNSDNPTVEEDWADGYNIIVGGNSLGRGLTFNNLQTVFYVRESKRPQADTLWQHARMFGYKRHKDTMRVFMPATIAQTFQEVYLGNEAIKNQLDHGTHINDIRVILGDGVAPTRANVLDKRKVGNLSGGVNYFAADPRIKNVEALDKKLLAYLDKHGEDSTIGMRAIITILNAFTVDPNDLDLATFKAALLDFERNQPHLTARMVLRTNRKVNQGTGALLSPTDQALSRAEVAHPLLILYRIEGVNDAAAQRGEPTWSSDPIWVPNIKLPGQRQFWCVDG

SEQ ID NO: 13 - DNA, 555 bp, unidentified
  misc_feature 1..555: note = Orotate phosphoribosyltransferase pyrE; CGL_RS13820, ncgl2676, cgl2773
  source 1..555: mol_type = other DNA
atgtcatctaattccattaacgcagaagcgcgcgctgagcttgctgaactgatcaaagagctagctgtcgtccacggtgaagtcaccttgtcttcgggcaagaaggctgattactacatcgatgtccgtcgtgccaccttgcacgcgcgcgcatctcgcctgatcggtcagctgctgcgcgaagccaccgctgactgggactatgacgcagttggcggcctgaccttgggcgctgacccggttgccaccgccatcatgcacgccgacggccgcgatatcaacgcgtttgtggtgcgcaaggaggccaagaagcacggcatgcagcgtcgcattgagggccctgacctgacgggcaagaaggtgctcgtggtggaagataccaccaccaccggaaattcccctctgacagctgttgccgcgttgcgtgaagctggcattgaggttgtgggcgttgccaccgtggtcgatcgcgcaaccggtgcagatgaggttatcgcagcggaaggccttccttaccgcagcttgctgggactttctgatcttggactcaactaa

SEQ ID NO: 14 - protein, 184 aa, unidentified
  REGION 1..184: note = Orotate phosphoribosyltransferase pyrE; CGL_RS13820, ncgl2676, cgl2773
MSSNSINAEARAELAELIKELAVVHGEVTLSSGKKADYYIDVRRATLHARASRLIGQLLREATADWDYDAVGGLTLGADPVATAIMHADGRDINAFVVRKEAKKHGMQRRIEGPDLTGKKVLVVEDTTTTGNSPLTAVAALREAGIEVVGVATVVDRATGADEVIAAEGLPYRSLLGLSDLGLN

SEQ ID NO: 15 - DNA, 2085 bp, unidentified
  misc_feature 1..2085: note = 5'-nucleotidase ushA; cgl0328- CGL_RS01710, ncgl0322, cg0397
  source 1..2085: mol_type = other DNA
atgaagaggctttcccgtgcagccctcgcagtggtcgccaccaccgcagttagcttcagcgcactcgcagttccagctttcgcagacgaagcaagcaatgttgagctcaacatcctcggtgtcaccgacttccacggacacatcgagcagaaggctgttaaagatgataagggagtaatcaccggttactcagaaatgggtgccagtggcgttgcctgctacgtcgacgctgaacgcgcggacaacccaaacacccgcttcatcaccgttggtgacaacattggtggatccccattcgtgtcctccatcctgaaggatgagccaaccttgcaagccctcagcgccatcggtgttgacgcatccgcactgggcaatcacgaattcgaccagggctactcagacctggtgaaccgcgtttccctcgacggctccggcagcgcaaagttcccatacctcggcgcaaacgttgaaggtggcaccccagcacctgcaaagtctgaaatcatcgagatggacggcgtcaagatcgcttacgtcggcgcagtaaccgaggagaccgcaaccttggtctccccagcaggcatcgaaggcatcaccttcaccggcgacatcgacgctatcaacgcagaagcagatcgcgtcattgaggcaggcgaagcagacgtagtcatcgcattgatccacgctgaagccgctccaaccgatctattctccaacaacgttgacgttgtattctccggacacacccacttcgactacgttgctgaaggcgaagcacgtggcgacaagcagccactcgttgtcatccagggccacgaatacggcaaggtcatctccgacgtggagatctcctacgaccgcgaagcaggcaagatcaccaacattgaggcgaagaatgtctctgctactgacgttgtggaaaactgtgagactccaaacacagcagtcgacgcaatcgttgcagctgctgttgaggccgctgaagaagcaggtaatgaagttgttgcaaccattgacaacggcttctaccgtggggcggatgaagagggtacgaccggctccaaccgtggtgttgagtcttccctgagcaacctcatcgcagaagctggactgtgggcagtcaacgacgcgaccatcctgaacgctgacatcggcatcatgaacgcaggcggcgtgcgtgcggacctcgaagcaggcgaagttaccttcgcagatgcatacgcaacccagaacttctccaacacctacggcgtacgtgaagtgtctggtgcgcagttcaaagaagcactggaacagcagtggaaggaaaccggcgaccgcccacgtctggcattgggactgtccagcaacgtccagtactcctacgacgagacccgcgaatacggcgaccgcatcacccacatcaccttcaacggtgagccaatggatatgaaggagacctaccgcgtcacaggatcatccttcctgctcgcaggtggcgactccttcactgcattcgctgaaggcggcccaatcgctgaaaccggcatggttgacattgacctgttcaacaactacatcgcagctcacccagatgcaccaattcgtgcaaatcagagctcagtaggcatcgccctttccggcccggcagttgcagaagacggaactttggtccctggtgaagagctgaccgtcgatctttcttccctctcctacaccggacctgaagctaagccaaccaccgttgaggtgaccgttggtactgagaagaagactgcggacgtcgataacaccatcgttcctcagtttgacagcaccggcaaggcaactgtcaccctgactgttcctgagggagctacctctgtcaagatcgcaactgacaatggcactacctttgaactgccagtaaccgtaaacggtgaaggcaacaatgatgacgatgatgataaggagcagcagtcctccggatcctccgacgccggttcccttgtagcagttctcggtgttcttggagcactcggtggcctggtggcgttcttcctgaactctgcgcagggcgcaccattcttggctcagcttcaggctatgtttgcgcagttcatgtaa

SEQ ID NO: 16 - protein, 694 aa, unidentified
  REGION 1..694: note = 5'-nucleotidase ushA; cgl0328- CGL_RS01710, ncgl0322, cg0397
MKRLSRAALAVVATTAVSFSALAVPAFADEASNVELNILGVTDFHGHIEQKAVKDDKGVITGYSEMGASGVACYVDAERADNPNTRFITVGDNIGGSPFVSSILKDEPTLQALSAIGVDASALGNHEFDQGYSDLVNRVSLDGSGSAKFPYLGANVEGGTPAPAKSEIIEMDGVKIAYVGAVTEETATLVSPAGIEGITFTGDIDAINAEADRVIEAGEADVVIALIHAEAAPTDLFSNNVDVVFSGHTHFDYVAEGEARGDKQPLVVIQGHEYGKVISDVEISYDREAGKITNIEAKNVSATDVVENCETPNTAVDAIVAAAVEAAEEAGNEVVATIDNGFYRGADEEGTTGSNRGVESSLSNLIAEAGLWAVNDATILNADIGIMNAGGVRADLEAGEVTFADAYATQNFSNTYGVREVSGAQFKEALEQQWKETGDRPRLALGLSSNVQYSYDETREYGDRITHITFNGEPMDMKETYRVTGSSFLLAGGDSFTAFAEGGPIAETGMVDIDLFNNYIAAHPDAPIRANQSSVGIALSGPAVAEDGTLVPGEELTVDLSSLSYTGPEAKPTTVEVTVGTEKKTADVDNTIVPQFDSTGKATVTLTVPEGATSVKIATDNGTTFELPVTVNGEGNNDDDDDKEQQSSGSSDAGSLVAVLGVLGALGGLVAFFLNSAQGAPFLAQLQAMFAQFM

SEQ ID NO: 17 - DNA, 693 bp, unidentified
  misc_feature 1..693: note = Nicotinamide ribose transporter pnuC; ncgl0063, CGL_RS00355, cgl0064
  source 1..693: mol_type = other DNA
atgaatcctataaccgaattattagacgcaacactatggatcggcggagttccgattctgtggcgcgaaatcatcggcaacgttttcggattatttagcgcgtgggcaggaatgcgacgcatcgtgtgggcatggcccatcggcatcataggcaacgcgctgctgttcacagtatttatgggcggccttttccacactccacaaaacctcgatctctacggccaagcgggtcgccagatcatgttcatcatcgtcagtggttatggctggtaccaatggtcggccgcaaaacgtcgcgcactcaccccagaaaatgcagtagcagtggttcctcgctgggcaagcaccaaagaacgcgccggcattgtgattgcggcggttgtgggaacactcagctttgcctggattttccaagcactcggctcctgggggccatgggccgacgcgtggattttcgtcggctcaatcctggctacctacggaatggctcgcggatggacagagttctggctgatctggatcgccgtcgacatagttggcgttcctctacttttgactgctggctactacccatccgcggtgctttacctggtgtacggtgcgtttgtcagctggggatttgtcgtgtggctgcgggtgcaaaaagcagacaaggctcgtgcgctggaagctcaggagtctgtgacagtctga

SEQ ID NO: 18 - protein, 230 aa, unidentified
  REGION 1..230: note = Nicotinamide ribose transporter pnuC; ncgl0063, CGL_RS00355, cgl0064
MNPITELLDATLWIGGVPILWREIIGNVFGLFSAWAGMRRIVWAWPIGIIGNALLFTVFMGGLFHTPQNLDLYGQAGRQIMFIIVSGYGWYQWSAAKRRALTPENAVAVVPRWASTKERAGIVIAAVVGTLSFAWIFQALGSWGPWADAWIFVGSILATYGMARGWTEFWLIWIAVDIVGVPLLLTAGYYPSAVLYLVYGAFVSWGFVVWLRVQKADKARALEAQESVTV

SEQ ID NO: 19 - DNA, 939 bp, unidentified
  misc_feature 1..939: note = Purine nucleosidase iunH3; cgl1364 (CGL_RS06810, ncgl1309, cg1543, iunH3)
  source 1..939: mol_type = other DNA
atgaccaccaagatcatcctcgactgcgatccaggacacgacgacgctgtagccatgctgctcgcagccggcagcccagaaattgaactgcttggaatcaccacggtcggcggcaaccagaccttggacaaggtcacccacaatacgcaggtcgtagccaccatcgctgatatcaatgcgcccatctaccgcggtgtcacccgaccattggtgcgccccgttgaggtagccgaagatatccacggcgataccggcatggaaatccacaagtacgaactgcctgaaccaaccaagcaggtagaagacacccacgcggtggatttcatcatcgataccatcatgaataacgagcccggcagcgtagcgctggttcccaccggaccactgaccaacatcgcgctggcagtccggaaagaaccacgcatcgccgagcgagtcaaggaagttgtcctcatgggcgggggctaccacgtaggaaactggaccgccgtagctgaattcaacatcaagatcgaccccgaagcagcccacatcgtattcaacgaaaagtggccactgactatggtcggcctcgaccttacccaccaggcgctcgcaacacctgagatcgaagccaagttcaacgagctgggcaccgacgtcgccgacttcgtcgtcgcgcttttcgacgctttccgcaagaattaccaggacgcacagggttttgataacccaccagtacacgacccttgtgctgttgcataccttgttgacccaaccgtattcaccacccgcaaagcaccactcgatgtggagctgtacggcgcactcaccacaggcatgaccgttgctgatttccgcgcaccggctccagcagattgcaccacccaagtagctgttgacctggactttgataaattctggaacatggtgatcgatgcagtaaagcgcatcggatag

SEQ ID NO: 20 - protein, 312 aa, unidentified
  REGION 1..312: note = Purine nucleosidase iunH3; cgl1364 (CGL_RS06810, ncgl1309, cg1543, iunH3)
MTTKIILDCDPGHDDAVAMLLAAGSPEIELLGITTVGGNQTLDKVTHNTQVVATIADINAPIYRGVTRPLVRPVEVAEDIHGDTGMEIHKYELPEPTKQVEDTHAVDFIIDTIMNNEPGSVALVPTGPLTNIALAVRKEPRIAERVKEVVLMGGGYHVGNWTAVAEFNIKIDPEAAHIVFNEKWPLTMVGLDLTHQALATPEIEAKFNELGTDVADFVVALFDAFRKNYQDAQGFDNPPVHDPCAVAYLVDPTVFTTRKAPLDVELYGALTTGMTVADFRAPAPADCTTQVAVDLDFDKFWNMVIDAVKRIG

SEQ ID NO: 21 - DNA, 951 bp, unidentified
  misc_feature 1..951: note = Purine nucleosidase iunH2; cgl1977 (cg2168, iunH2, CYL77_RS09970)
  source 1..951: mol_type = other DNA
atgagcaaaaaagccatccttgatatcgacaccggcatcgatgatgccctcgcacttgcctacgcactgggctcacctgaactagagctcattggtgtcaccaccacctacggtaacgtgctactcgaaaccggtgcagtcaatgacctggcactgcttgatctgttcggtgcaccagaagtacctgtgtacttgggtgagccacacgcacagaccaaggatggctttgaagttcttgagatctccgcgttcattcacggacaaaacggcatcggcgaagtcgagctgccagcaagcgagtcaaaggcactccccggcgcagtggatttcctcattgattccgtcaacacccacggcgatgacctggtgatcatcgcaactggtcccatgaccaacctgtctgcggcaatcgcaaaggatccaagctttgcttccaaggctcacgtggtcatcatgggtggcgccttgactgtcccaggcaacgtcagcacatgggcagaagcaaacatcaaccaggacccagatgcagcaaacgatctgttccgttccggtgcagatgtcaccatgatcggtcttgatgtcaccctgcagacccttcttaccaagaagcacactgcgcagtggcgcgaactgggcactccagctgctatcgcactggccgacatgactgattactacatcaaggcatatgagaccaccgcaccacacctgggcggttgcggcctgcacgacccactggcagtaggcgttgcagtggacccaagcctggtcactttgctccccatcaacctcaaggtagacattgagggcgagacccgtggacgcaccattggcgatgaagtccgcctcaacgatccagtgcgcacctcccgcgcagctgtcgccgtagacgtggatcgtttcctttctgaattcatgacccgcatcggccgagtcgcagcacagcagtaa

SEQ ID NO: 22 - protein, 316 aa, unidentified
  REGION 1..316: note = Purine nucleosidase iunH2; cgl1977 (cg2168, iunH2, CYL77_RS09970)
MSKKAILDIDTGIDDALALAYALGSPELELIGVTTTYGNVLLETGAVNDLALLDLFGAPEVPVYLGEPHAQTKDGFEVLEISAFIHGQNGIGEVELPASESKALPGAVDFLIDSVNTHGDDLVIIATGPMTNLSAAIAKDPSFASKAHVVIMGGALTVPGNVSTWAEANINQDPDAANDLFRSGADVTMIGLDVTLQTLLTKKHTAQWRELGTPAAIALADMTDYYIKAYETTAPHLGGCGLHDPLAVGVAVDPSLVTLLPINLKVDIEGETRGRTIGDEVRLNDPVRTSRAAVAVDVDRFLSEFMTRIGRVAAQQ

SEQ ID NO: 23 - DNA, 905 bp, unidentified
  misc_feature 1..905: note = Purine nucleosidase iunH1; cgl2835 (cg3137, iunH1, CYL77_RS14340)
  source 1..905: mol_type = other DNA
atgattcctgttctcatcgactgcgacaccggcatcgacgacgccctcgccctgatctacctggttgctttgcataaacgtggtgaaatccaactttttggagcaacgaccaccgcaggaaatgttgatgtgaaacaaaccgcctcaataccaggtgggtgttggatcagtgtggattagcggacatcccggtcctcgcaggacaacctgaaccaaagcacgtgccgctagtgactactccagaaacacacggcgaccatggccttggttatataaacccaggtcacgtcgaaattccagaaggtgactggaagcagctgtggaaagaacacctcagtaacccagaaactaagctgattgtcaccgggcccgccaccaaccttgcggaattcgggccagtggaaaacgtcacgctgatgggtggcacctacctttatccaggcaacaccactccaacggcagaatggaatacctgggttgatccacacggagctaaagaagcattcgcggcagcccaaaagcccattacggtgtgttccttgggcgtgaccgagcagtttacgctgaacccggacatcctttctacacttatcaacacgcttggcagccaacccatcgcagagcatttacctgagatgctgcgcttttactttgaatttcacgaagtgcagggcgaaggttaccttgctcaaattcatgacctgctgacctgcatgattgccttggataaaatcccattttcaggccgtgaagtaaccgtggacgtggaggctgattcgcccttgatgcgtggcaccactgttgcagatattcgcggacattggggcaagccagctaacgcatttcttgtggaaaccgcagacattgaggccgcccacgcggaacttctaagagcagtggaatga

SEQ ID NO: 24 - protein, 301 aa, unidentified
  REGION 1..301: note = Purine nucleosidase iunH1; cgl2835 (cg3137, iunH1, CYL77_RS14340)
MIPVLIDCDTGIDDALALIYLVALHKRGEIQLFGATTTAGNVDVKQTAINTRWVLDQCGLADIPVLAGQPEPKHVPLVTTPETHGDHGLGYINPGHVEIPEGDWKQLWKEHLSNPETKLIVTGPATNLAEFGPVENVTLMGGTYLYPGNTTPTAEWNTWVDPHGAKEAFAAAQKPITVCSLGVTEQFTLNPDILSTLINTLGSQPIAEHLPEMLRFYFEFHEVQGEGYLAQIHDLLTCMIALDKIPFSGREVTVDVEADSPLMRGTTVADIRGHWGKPANAFLVETADIEAAHAELLRAVE

SEQ ID NO: 25 - DNA, 1623 bp, unidentified
  misc_feature 1..1623: note = Glucose-6P isomerase pgi; cgl0851 (ncgl0817)
  source 1..1623: mol_type = other DNA
atggcggacatttcgaccacccaggtttggcaagacctgaccgatcattactcaaacttccaggcaaccactctgcgtgaacttttcaaggaagaaaaccgcgccgagaagtacaccttctccgcggctggcctccacgtcgacctgtcgaagaatctgcttgacgacgccaccctcaccaagctccttgcactgaccgaagaatctggccttcgcgaacgcattgacgcgatgtttgccggtgaacacctcaacaacaccgaagaccgcgctgtcctccacaccgcgctgcgccttcctgccgaagctgatctgtcagtagatggccaagatgttgctgctgatgtccacgaagttttgggacgcatgcgtgacttcgctactgcgctgcgctcaggcaactggttgggacacaccggccacacgatcaagaagatcgtcaacattggtatcggtggctctgacctcggaccagccatggctacgaaggctctgcgtgcatacgcgaccgctggtatctcagcagaattcgtctccaacgtcgacccagcagacctcgtttctgtgttggaagacctcgatgcagaatccacattgttcgtgatcgcttcgaaaactttcaccacccaggagacgctgtccaacgctcgtgcagctcgtgcttggctggtagagaagctcggtgaagaggctgtcgcgaagcacttcgtcgcagtgtccaccaatgctgaaaaggtcgcagagttcggtatcgacacggacaacatgttcggcttctgggactgggtcggaggtcgttactccgtggactccgcagttggtctttccctcatggcagtgatcggccctcgcgacttcatgcgtttcctcggtggattccacgcgatggatgaacacttccgcaccaccaagttcgaagagaacgttccaatcttgatggctctgctcggtgtctggtactccgatttctatggtgcagaaacccacgctgtcctaccttattccgaggatctcagccgttttgctgcttacctccagcagctgaccatggaatcaaatggcaagtcagtccaccgcgacggctcccctgtttccactggcactggcgaaatttactggggtgagcctggcacaaatggccagcacgctttcttccagctgatccaccagggcactcgccttgttccagctgatttcattggtttcgctcgtccaaagcaggatcttcctgccggtgagcgcaccatgcatgaccttttgatgagcaacttcttcgcacagaccaaggttttggctttcggtaagaacgctgaagagatcgctgcggaaggtgtcgcacctgagctggtcaaccacaaggtcatgccaggtaatcgcccaaccaccaccattttggcggaggaacttaccccttctattctcggtgcgttgatcgctttgtacgaacacatcgtgatggttcagggcgtgatttgggacatcaactccttcgaccaatggggtgttgaactgggcaaacagcaggcaaatgacctcgctccggctgtctctggtgaagaggatgttgactcgggagattcttccactgattcactgattaagtggtaccgcgcaaataggtag

SEQ ID NO: 26 - protein, 540 aa, unidentified
  REGION 1..540: note = Glucose-6P isomerase PGI; cgl0851 (ncgl0817)
MADISTTQVWQDLTDHYSNFQATTLRELFKEENRAEKYTFSAAGLHVDLSKNLLDDATLTKLLALTEESGLRERIDAMFAGEHLNNTEDRAVLHTALRLPAEADLSVDGQDVAADVHEVLGRMRDFATALRSGNWLGHTGHTIKKIVNIGIGGSDLGPAMATKALRAYATAGISAEFVSNVDPADLVSVLEDLDAESTLFVIASKTFTTQETLSNARAARAWLVEKLGEEAVAKHFVAVSTNAEKVAEFGIDTDNMFGFWDWVGGRYSVDSAVGLSLMAVIGPRDFMRFLGGFHAMDEHFRTTKFEENVPILMALLGVWYSDFYGAETHAVLPYSEDLSRFAAYLQQLTMESNGKSVHRDGSPVSTGTGEIYWGEPGTNGQHAFFQLIHQGTRLVPADFIGFARPKQDLPAGERTMHDLLMSNFFAQTKVLAFGKNAEEIAAEGVAPELVNHKVMPGNRPTTTILAEELTPSILGALIALYEHIVMVQGVIWDINSFDQWGVELGKQQANDLAPAVSGEEDVDSGDSSTDSLIKWYRANR

SEQ ID NO: 27 - DNA, 1545 bp, unidentified
  misc_feature 1..1545: note = cgZWF codon-optimized mutant (A243T) of the of the glucose-6-phosphate dehydrogenase from Corynebacterium glutamicum (cg1778, Cgl1576, NCgl1514)
  source 1..1545: mol_type = other DNA
atgagtaccaacaccaccccgtcaagctggacaaatccattgcgcgacccccaggataagcgcttgccccgcatcgcaggaccctccggcatggtcatttttggggtgaccggcgatctggcacgcaagaaactgctaccagccatctatgacttggcaaatcgcggcttactgccacctggcttctctctcgtgggctatggtcgccgtgaatggtctaaggaggacttcgaaaagtacgttcgtgatgcagcgtccgcgggagcccgaacggaatttcgtgaaaacgtctgggaacgccttgcagaaggcatggaatttgtccgcggaaattttgatgatgacgccgcattcgacaacttggcggcgacgctgaagcgcatcgataagacgagaggcactgctggtaactgggcgtactatctgtccatcccaccggactcctttacggcggtgtgccaccagctagagcgttccggcatggctgagtccaccgaagaggcatggcgccgagtgatcattgaaaagccattcgggcacaacctggaatcggcacacgagctcaaccaactggtcaacgccgttttcccggagtcatcagtgtttagaatcgatcactacctgggtaaagaaaccgtgcagaatatcctcgcgctgcgattcgcaaatcaactttttgaacccctttggaacagcaactatgtcgatcacgtccaaattaccatgactgaagatattggcttgggaggacgcgcgggttattatgatggaatcggagcagcgcgcgacgtcatccagaatcacctcattcagctgttggcgctggtagcgatggaggaacccattagctttgtgcctgctcagctgcaagcagaaaagatcaaagttctgagcgctaccaaaccttgttaccctctggataagacctcagctcgcggtcaatatgctgctggctggcaaggatctgagctggtcaagggccttcgtgaagaggacggtttcaaccccgagagcaccacggaaaccttcgccgcatgtacccttgaaatcacaagtcgccgctgggccggcgtcccattctacctgcgtactggcaagagactcggccgacgagttacagagatcgctgttgtgtttaaagatgctccccaccagccgtttgatggagacatgaccgtttcccttggccaaaatgcgatcgtaattcgcgtacaaccagacgagggtgttcttatccgctttggttccaaggtgcccggttccgctatggaggttcgtgacgttaatatggacttcagctatagcgaatccttcaccgaagagtcacctgaagcatacgaacgcctgatcctggatgccctcctggacgagtccagcttgtttccaaccaacgaggaagtggaactgtcttggaaaatcctggacccaattctggaagcttgggatgccgatggcgaaccggaggactacccagctgggacctgggggccaaaatcggcggatgagatgttatcccgtaacggccacacatggcgccgaccttga

SEQ ID NO: 28 - protein, 514 aa, unidentified
  REGION 1..514: note = cgZWF mutant (A243T) of the of the glucose-6-phosphate dehydrogenase from Corynebacterium glutamicum (cg1778, Cgl1576, NCgl1514)
MSTNTTPSSWTNPLRDPQDKRLPRIAGPSGMVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFTAVCHQLERSGMAESTEEAWRRVIIEKPFGHNLESAHELNQLVNAVFPESSVFRIDHYLGKETVQNILALRFANQLFEPLWNSNYVDHVQITMAEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 29 - DNA, 1461 bp, unidentified
  misc_feature 1..1461: note = Glucose-6-phosphate dehydrogenase of Leuconoston mesenteroides, codon optimized mutant (R46E/Q47E) of the gene having accession number M64446.1; lmZWF
  source 1..1461: mol_type = other DNA
atggtttccgaaataaagaccctcgttactttctttggcggcaccggtgaccttgcaaaacgcaagctctacccctctgtattcaacctgtacaaaaaagggtatctgcaaaaacacttcgccattgttggtaccgctgaagaggcgctaaacgacgacgagttcaaacagcttgtccgtgattccattaaagacttcaccgatgaccaagcccaggcagaggccttcatcgaacatttttcttatcgagcacacgatgtgaccgatgccgcatcgtatgcagtcctgaaggaagcgatcgaggaggcggccgataagttcgatattgacggtaaccgcatattttacatgtcggtggcaccacgcttcttcggtaccatcgctaaatatctgaagtccgagggtctgcttgctgatactggctacaatcggctgatgattgaaaagccttttggaacctcttatgacacagccgcagaactacagaatgacttggagaacgctttcgatgataatcagcttttccgtatcgatcattatctgggtaaagaaatggtccagaatatcgcagctctgcgcttcggaaaccctatattcgacgctgcgtggaacaaggattacatcaagaacgttcaagttacactctccgaagtgttgggggttgaagagcgagccggctactatgacaccgccggagctctacttgatatgatccagaaccacacgatgcagatcgttggctggctcgccatggaaaaaccagagtccttcaccgataaagacatccgcgcggctaagaacgccgcttttaatgccctgaaaatctacgacgaggctgaagtgaacaaatattttgtgcgtgcccaatatggtgctggagattctgccgatttcaaaccttacttagaggaactcgatgtcccagcagattccaaaaacaacaccttcattgcaggcgaattacagtttgatcttccacgttgggaaggtgtgcctttttacgtgcgcagcggtaaacgactcgcagccaaacagactcgcgtcgatattgttttcaaggctggcacctttaatttcgggtctgaacaggaagctcaagaggccgttctgtccatcatcattgatccgaagggagcaatcgaactgaagctcaatgcaaaatctgtggaagatgctttcaacacccgcactatcgacttgggctggaccgtgagcgatgaggacaagaaaaatacgccagaaccttatgaaaggatgatccacgatacgatgaacggtgacggcagcaacttcgcagattggaatggcgtgagcattgcgtggaagttcgtagatgctatttctgcggtatatacggccgataaggccccgcttgaaacctacaagtcgggctccatgggacccgaagccagcgacaaattgctcgccgcaaacggagatgcatgggtattcaaggggtag

SEQ ID NO: 30 - protein, 486 aa, unidentified
  REGION 1..486: note = Mutant (R46E/Q47E) of the glucose-6-phosphate dehydrogenase of Leuconoston.mesenteroides; lmZWF
MVSEIKTLVTFFGGTGDLAKRKLYPSVFNLYKKGYLQKHFAIVGTAEEALNDDEFKQLVRDSIKDFTDDQAQAEAFIEHFSYRAHDVTDAASYAVLKEAIEEAADKFDIDGNRIFYMSVAPRFFGTIAKYLKSEGLLADTGYNRLMIEKPFGTSYDTAAELQNDLENAFDDNQLFRIDHYLGKEMVQNIAALRFGNPIFDAAWNKDYIKNVQVTLSEVLGVEERAGYYDTAGALLDMIQNHTMQIVGWLAMEKPESFTDKDIRAAKNAAFNALKIYDEAEVNKYFVRAQYGAGDSADFKPYLEELDVPADSKNNTFIAGELQFDLPRWEGVPFYVRSGKRLAAKQTRVDIVFKAGTFNFGSEQEAQEAVLSIIIDPKGAIELKLNAKSVEDAFNTRTIDLGWTVSDEDKKNTPEPYERMIHDTMNGDGSNFADWNGVSIAWKFVDAISAVYTADKAPLETYKSGSMGPEASDKLLAANGDAWVFKG

SEQ ID NO: 31 - DNA, 1458 bp, Zymomonas mobilis
  misc_feature 1..1458: note = Codon optimized variant of the glucose-6-phosphate dehydrogenase from Zymomonas mobilis strain ATCC 10988 (Zmob_0908)
  source 1..1458: mol_type = genomic DNA
atgactaatactgtttctaccatgatccttttcggcagcaccggagatctctcgcagcgcatgcttcttccctcgctgtacgggctggatgcagacggtctactcgccgacgacctccgcattgtgtgtacctctcgttccgagtacgataccgacggatttcgtgattttgctgagaaggcactggaccgtttcgttgcctccgacagacttaatgatgatgcaaaagcgaagttcctcaacaagcttttctacgcaacggttgacatcaccgatccaacccaatttggaaagctcgcagacctctgcggtccagtcgaaaagggcattgcaatctacctttccacagcaccatccttgttcgaaggcgcaattgctggcttgaaacaggcgggcctggccggcccgacctcccgccttgcattggaaaagcccttgggtcaagatcttgcttcctctgatcacatcaacgacgcagtgctgaaggttttttccgaaaaacaagtataccgtatcgaccactatcttgggaaagaaaccgtccagaatctcctaacactccgctttggaaatgcattgttcgagccgttgtggaactcaaaggggattgaccacgtgcagatctccgtcgctgagacagtgggactcgaaggacgcatcggctactttgacggctccggctccctgcgagacatggtgcagtctcacatcctgcaattggttgcccttgtagctatggagcccccggctcacatggaagcaaacgcggtccgcgacgaaaaggttaaggtgttccgtgcacttcgtcccattaacaacgacactgttttcacacacaccgtgactggccaatacggcgccggcgtgtcggggggaaaggaagttgcaggctacatcgatgagcttggacaaccgagtgatactgaaacctttgttgcaattaaagcacacgtggataactggcgctggcagggagttcccttctacatccgcactggtaaacggctccctgcccgccgttcagagatcgtcgttcagttcaaaccagttccccactccattttttcaagctcaggaggaatccttcagcctaataaattgcgcattgtcctgcaaccagacgaaaccatccaaatctcaatgatggtcaaggaaccaggtcttgacagaaatggtgcacacatgcgtgaggtctggctggatctctctttgaccgacgtgttcaaagatcgaaagcgccggattgcttacgagcgccttatgctcgatctgattgagggtgacgcaaccctcttcgtgcgccgcgacgaggtcgaggcacagtgggtttggatcgacggtatccgggaaggctggaaggctaatagcatgaagcctaaaacctatgtctccggcacctggggaccctccaccgctattgcattggcagagcgcgatggcgtcacctggtacgactaa

SEQ ID NO: 32 - protein, 485 aa, Zymomonas mobilis
  REGION 1..485: note = Codon-optimized variant of the glu6-phosphate dehydrogenase from Zymomonas mobilis strain ATCC 10988 (NCBI-ProteinID: AEH62743.1)
MTNTVSTMILFGSTGDLSQRMLLPSLYGLDADGLLADDLRIVCTSRSEYDTDGFRDFAEKALDRFVASDRLNDDAKAKFLNKLFYATVDITDPTQFGKLADLCGPVEKGIAIYLSTAPSLFEGAIAGLKQAGLAGPTSRLALEKPLGQDLASSDHINDAVLKVFSEKQVYRIDHYLGKETVQNLLTLRFGNALFEPLWNSKGIDHVQISVAETVGLEGRIGYFDGSGSLRDMVQSHILQLVALVAMEPPAHMEANAVRDEKVKVFRALRPINNDTVFTHTVTGQYGAGVSGGKEVAGYIDELGQPSDTETFVAIKAHVDNWRWQGVPFYIRTGKRLPARRSEIVVQFKPVPHSIFSSSGGILQPNKLRIVLQPDETIQISMMVKEPGLDRNGAHMREVWLDLSLTDVFKDRKRRIAYERLMLDLIEGDATLFVRRDEVEAQWVWIDGIREGWKANSMKPKTYVSGTWGPSTAIALAERDGVTWYD

SEQ ID NO: 33 - DNA, 1401 bp, Escherichia coli
  misc_feature 1..1401: note = Soluble Pyridine Nucleotide Transhydrogenase from Escherichia coli; udhA (NP_418397.2, EG11428)
  source 1..1401: mol_type = genomic DNA
atgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcctggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaa

SEQ ID NO: 34 - protein, 466 aa, Escherichia coli
  REGION 1..466: note = Soluble Pyridine Nucleotide Transhydrogenase from Escherichia coli; udhA (AAC76944)
MPHSYDYDAIVIGSGPGGEGAAMGLVKQGARVAVIERYQNVGGGCTHWGTIPSKALRHAVSRIIEFNQNPLYSDHSRLLRSSFADILNHADNVINQQTRMRQGFYERNHCEILQGNARFVDEHTLALDCLDGSVETLTAEKFVIACGSRPYHPTDVDFTHPRIYDSDSILSMHHEPRHVLIYGAGVIGCEYASIFRGMDVKVDLINTRDRLLAFLDQEMSDSLSYHFWNSGVVIRHNEEYEKIEGCDDGVIMHLKSGKKLKADCLLYANGRTGNTDSLALQNIGLETDSRGQLKVNSMYQTAQPHVYAVGDVIGYPSLASAAYDQGRIAAQALVKGEATAHLIEDIPTGIYTIPEISSVGKTEQQLTAMKVPYEVGRAQFKHLARAQIVGMNVGTLKILFHRETKEILGIHCFGERAAEIIHIGQAIMEQKGGGNTIEYFVNTTFNYPTMAEAYRVAALNGLNRLF

SEQ ID NO: 35 - DNA, 1401 bp, Escherichia coli; identical to SEQ ID NO: 33
  misc_feature 1..1401: note = "A" subunit of Membrane bound Pyridine Nucleotide Transhydrogenase (pnt) from Escherichia coli MG1655; ECK1598 (variant g1342a, NP_416120.1)
  source 1..1401: mol_type = genomic DNA

SEQ ID NO: 36 - protein, 510 aa, Escherichia coli
  REGION 1..510: note = "A" subunit of Membrane bound Pyridine Nucleotide Transhydrogenase (pnt) from Escherichia coli; P07001.2 (A434T variant)
MRIGIPRERLTNETRVAATPKTVEQLLKLGFTVAVESGAGQLASFDDKAFVQAGAEIVEGNSVWQSEIILKVNAPLDDEIALLNPGTTLVSFIWPAQNPELMQKLAERNVTVMAMDSVPRISRAQSLDALSSMANIAGYRAIVEAAHEFGRFFTGQITAAGKVPPAKVMVIGAGVAGLAAIGAANSLGAIVRAFDTRPEVKEQVQSMGAEFLELDFKEEAGSGDGYAKVMSDAFIKAEMELFAAQAKEVDIIVTTALIPGKPAPKLITREMVDSMKAGSVIVDLAAQNGGNCEYTVPGEIFTTENGVKVIGYTDLPGRLPTQSSQLYGTNLVNLLKLLCKEKDGNITVDFDDVVIRGVTVIRAGEITWPAPPIQVSAQPQAAQKAAPEVKTEEKCTCSPWRKYALMALAIILFGWMASVAPKEFLGHFTVFALTCVVGYYVVWNVSHALHTPLMSVTNAISGIIVVGALLQIGQGGWVSFLSFIAVLIASINIFGGFTVTQRMLKMFRKN

SEQ ID NO: 37 - DNA, 1389 bp, Escherichia coli
  misc_feature 1..1389: note = "B" subunit of Membrane bound Pyridine Nucleotide Transhydrogenase (pntB) from Escherichia coli (MG1655; ECK1597; NP_416119.1)
  source 1..1389: mol_type = genomic DNA
atgtctggaggattagttacagctgcatacattgttgccgcgatcctgtttatcttcagtctggccggtctttcgaaacatgaaacgtctcgccagggtaacaacttcggtatcgccgggatggcgattgcgttaatcgcaaccatttttggaccggatacgggtaatgttggctggatcttgctggcgatggtcattggtggggcaattggtatccgtctggcgaagaaagttgaaatgaccgaaatgccagaactggtggcgatcctgcatagcttcgtgggtctggcggcagtgctggttggctttaacagctatctgcatcatgacgcgggaatggcaccgattctggtcaatattcacctgacggaagtgttcctcggtatcttcatcggggcggtaacgttcacgggttcggtggtggcgttcggcaaactgtgtggcaagatttcgtctaaaccattgatgctgccaaaccgtcacaaaatgaacctggcggctctggtcgtttccttcctgctgctgattgtatttgttcgcacggacagcgtcggcctgcaagtgctggcattgctgataatgaccgcaattgcgctggtattcggctggcatttagtcgcctccatcggtggtgcagatatgccagtggtggtgtcgatgctgaactcgtactccggctgggcggctgcggctgcgggctttatgctcagcaacgacctgctgattgtgaccggtgcgctggtcggttcttcgggggctatcctttcttacattatgtgtaaggcgatgaaccgttcctttatcagcgttattgcgggtggtttcggcaccgacggctcttctactggcgatgatcaggaagtgggtgagcaccgcgaaatcaccgcagaagagacagcggaactgctgaaaaactcccattcagtgatcattactccggggtacggcatggcagtcgcgcaggcgcaatatcctgtcgctgaaattactgagaaattgcgcgctcgtggtattaatgtgcgtttcggtatccacccggtcgcggggcgtttgcctggacatatgaacgtattgctggctgaagcaaaagtaccgtatgacatcgtgctggaaatggacgagatcaatgatgactttgctgataccgataccgtactggtgattggtgctaacgatacggttaacccggcggcgcaggatgatccgaagagtccgattgctggtatgcctgtgctggaagtgtggaaagcgcagaacgtgattgtctttaaacgttcgatgaacactggctatgctggtgtgcaaaacccgctgttcttcaaggaaaacacccacatgctgtttggtgacgccaaagccagcgtggatgcaatcctgaaagctctgtaa

SEQ ID NO: 38 - protein, 462 aa, Escherichia coli
  REGION 1..462: note = "B" subunit of Membrane bound Pyridine Nucleotide Transhydrogenase (pntB) from Escherichia coli; P0AB69.1
MSGGLVTAAYIVAAILFIFSLAGLSKHETSRQGNNFGIAGMAIALIATIFGPDTGNVGWILLAMVIGGAIGIRLAKKVEMTEMPELVAILHSFVGLAAVLVGFNSYLHHDAGMAPILVNIHLTEVFLGIFIGAVTFTGSVVAFGKLCGKISSKPLMLPNRHKMNLAALVVSFLLLIVFVRTDSVGLQVLALLIMTAIALVFGWHLVASIGGADMPVVVSMLNSYSGWAAAAAGFMLSNDLLIVTGALVGSSGAILSYIMCKAMNRSFISVIAGGFGTDGSSTGDDQEVGEHREITAEETAELLKNSHSVIITPGYGMAVAQAQYPVAEITEKLRARGINVRFGIHPVAGRLPGHMNVLLAEAKVPYDIVLEMDEINDDFADTDTVLVIGANDTVNPAAQDDPKSPIAGMPVLEVWKAQNVIVFKRSMNTGYAGVQNPLFFKENTHMLFGDAKASVDAILKAL